(19) United States
(12) Patent Application Publication
(10) Pub. No.: US 2020/0064345 A1
(43) Pub. Date: Feb. 27, 2020

(54) METHODS FOR SCREENING INFECTIONS

(71) Applicant: HealthTell Inc., San Ramon, CA (US)

(72) Inventors: Kathryn Frances SYKES, San Ramon, CA (US); Robert William GERWIEN, San Ramon, CA (US); Jonathan Scott MELNICK, San Ramon, CA (US); Michael William ROWE, San Ramon, CA (US); Theodore Michael TARASOW, San Ramon, CA (US)

(21) Appl. No.: 16/488,078

(22) PCT Filed: Feb. 22, 2018
(86) PCT No.: PCT/US18/19287

§ 371 Date: Aug. 22, 2019

Related U.S. Application Data
(60) Provisional application No. 62/462,320, filed on Feb. 22, 2017.

(51) Int. Cl. G01N 33/569; C12Q 1/70; G01N 33/576

(52) U.S. Cl. CPC G01N 33/56905; C12Q 1/70; G01N 2469/20; G01N 33/5767; G01N 2333/183; G01N 33/5761

(57) ABSTRACT

The disclosed embodiments concern non-invasive methods, and apparatus, and systems for identifying infections. The methods are predicated on identifying discriminating peptides present on a peptide array, which are differentially bound by the different mixtures of antibodies present in samples from subjects consequent to an infection relative to binding of mixtures of antibodies present in reference subjects.

### CROSS-REFERENCE

This patent application claims the benefit of U.S. Provisional Patent Application No. 62/462,320, filed Feb. 22, 2017, which is incorporated herein by reference in its entirety.

### BACKGROUND

Infectious diseases are disorders usually caused by micro-organisms such as bacteria, viruses, fungi or parasites. Diagnosis of infection typically requires laboratory tests of body fluids such as blood, urine, throat swabs, stool samples, and in some cases, spinal taps. Imaging scan and biopsies may also be used to identify the infectious source. A variety of individual tests are available to diagnose an infection and include immunoassays, polymerase chain reaction, fluorescence in situ hybridization, and genetic testing for the pathogen. Present methods are time-consuming, complicated and labor-intensive and may require varying degrees of expertise. Additionally, the available diagnostic tools are often unreliable to detect early stages of infections, and often, more than one method is needed to positively diagnose an infection. In many instances, an infected person may not display any symptoms of infection until severe complications erupt.

An example is the infection by Trypanosoma cruzi (T. cruzi), which causes Chagas disease. Chagas disease is one of the leading cause of death and morbidity in Latin America and the Caribbean [Perez C J et al., Lymbery A J, Thompson R C (2014) Trends Parasitol 30: 176-182], and is a significant contributor to the global burden of cardiovascular disease [Chatelain E (2017) Comput Struct Biotechnol J 15: 98-103]. Chagas disease is considered the most neglected parasitic disease in these geographical regions, and epidemiologist are tracking its further spread into nonendemic countries including the US and Europe [Bern C (2015) Chagas' Disease. N Engl J Med 373: 1882; Bern C, and Montgomery S P (2009) Clin Infect Dis 49: e52-54; Rassi Jr A et al., (2010) The Lancet 375: 1388-1402]. The etiologic agent, T. cruzi, is a flagellated protozoan that is transmitted predominantly by blood-feeding triatomine insects to mammalian hosts, where it can multiply in any nucleated cell. Other modes of dissemination include blood transfusion or congenital and oral routes [Steverding D (2014) Parasit Vectors 7: 317].

Methods, diagnostic tools and additional biomarkers are needed to identify infections, preferably detect infections at early stages, and in the absence of symptoms.

### SUMMARY OF THE INVENTION

The disclosed embodiments concern methods, apparatus, and systems for identifying infections. The methods are predicated on identifying discriminating peptides present on a peptide array, which are differentially bound by biological samples from subjects consequent to an infection, as compared to binding of samples from reference subjects.

In one aspect a method is provided for identifying the serological state of a subject having or suspected of having a T. cruzi infection, the method comprising: (a) contacting said sample from said subject to an array of peptides comprising at least 10,000 different peptides; (b) detecting the binding of antibodies present in said sample to at least 25 peptides on said array to obtain a combination of binding signals; and (c) comparing said combination of binding signals to two or more groups of combinations of reference binding signals, wherein at least one of each of said group of combinations of reference binding signals are obtained from a plurality of reference subjects known to be seropositive for said infection, and wherein at least one of each of said group of combinations of reference binding signals are obtained from a plurality of subjects known to be seronegative for said infection, thereby determining the serological state of said subject. In some embodiments, the different peptides on the array are synthesized in situ. In some embodiments, the method further comprises (i) identifying a combination of differentiating reference binding signals wherein said differentiating binding signals distinguish samples from reference subjects known to be seropositive for said infection from samples from reference subjects known to be seronegative for said infection; and (ii) identifying a combination of discriminating peptides, wherein said discriminating peptides display signals corresponding to said differentiating reference binding signals. In some embodiments, each of said combination of differentiating reference binding signals is obtained by detecting the binding of antibodies present in a sample from each of said plurality of said reference subjects to at least 25 peptides on same arrays of peptides comprising at least 10,000 different peptides. In some embodiments, the different peptides on the array are synthesized in situ.

In some embodiments, the method provided identifies the serological state of a subject that is asymptomatic for said infection. In other embodiments, the method provided identifies the serological state of a subject that is symptomatic for said infection. In yet other embodiments, the method provided identifies the serological state of a subject that is symptomatic for any infection. In yet other embodiments, the discriminating peptides comprise one or more sequence motifs listed in FIG. 9B and FIGS. 23A-23C that are enriched in discriminating peptides among all peptides that contain the motif compared to discriminating peptides among all array peptides by greater than 100%. In yet other instances, the differentiating peptides are selected from the peptides listed in FIGS. 21A-N, Table 6 and Table 7.

In some embodiments, the discriminating peptides that are identified and that distinguish subjects that are seropositive from subjects that are seronegative for T. cruzi infection comprise one or more sequence motifs that are enriched by greater than 100%, including the sequence motifs listed in FIG. 9B. In some embodiments, the discriminating peptides are selected from the peptides listed, for example, in FIG. 21A-N. In other embodiments, the binding signal corresponding to the binding of antibodies in step (b) of the methods described herein is higher, for example, by about 25%, by about 30%, by about 40%, by about 50%, by about 60%, by about 70%, by about 80%, by about 90%, by about 100%, by about 125%, by about 150%, by about 175%, or by about 200% or more, than the reference binding signals obtained from the binding of antibodies from samples of subjects having a score of <1 when using the S/CO (signal to cut-off) serological scoring system for positively identifying Chagas disease patients.

In other embodiments, the methods and systems provided herein identifies the serological state of a subject having or suspected of having a T. cruzi infection relative to one or more groups of reference subjects that are seronegative for T. cruzii are seropositive for hepatitis B virus (HBV). The discriminating peptides that distinguish the subjects that are seropositive for T. cruzi from the subjects that are seropositive for HBV comprise one or more sequence motifs that are enriched by greater than 100%, including the sequence motifs listed in FIG. 14A.

In other embodiments, the methods and systems provided herein identifies the serological state of a subject having or suspected of having a T. cruzi infection relative to one or more groups of reference subjects that are seronegative for T. cruzii are seropositive for hepatitis C virus (HCV). The discriminating peptides that distinguish the subjects that are seropositive for T. cruzi from the subjects that are seropositive for HCV comprise sequence motifs that are enriched by greater than 100%, including the sequence motifs listed in FIG. 15A.

In other embodiments, the methods and systems provided herein identifies the serological state of a subject having or suspected of having a T. cruzi infection relative to one or more groups of reference subjects that are seronegative for T. cruzii are seropositive for West Nile Virus virus (WNV). The discriminating peptides that distinguish the subjects that are seropositive for T. cruzi from the subjects that are seropositive for WNV comprise sequence motifs that are enriched by greater than 100%, including the sequence motifs listed in FIG. 16A.

In another aspect, methods and systems are provided herein for identifying the serological state of a subject having or suspected of having a viral infection, said method comprising: (a) contacting said sample from said subject to an array of peptides comprising at least 10,000 different peptides; (b) detecting the binding of antibodies present in said sample to at least 25 peptides on said array to obtain a combination of binding signals; and (c) comparing said combination of binding signals to two or more groups of combinations of reference binding signals, wherein at least one of each of said group of combinations of reference binding signals are obtained from a plurality of reference subjects known to be seropositive for said infection, and wherein at least one of each of said group of combinations of reference binding signals are obtained from a plurality of subjects known to be seronegative for said infection, thereby determining the serological state of said subject. In some embodiments, the different peptides on the array are synthesized in situ. In some embodiments, the method further comprises (i) identifying a combination of differentiating reference binding signals wherein said differentiating binding signals distinguish samples from reference subjects known to be seropositive for said infection from samples from reference subjects known to be seronegative for said infection; and (ii) identifying a combination of discriminating peptides, wherein said discriminating peptides display signals corresponding to said differentiating reference binding signals.

In some embodiments, the methods and system described herein identifies the serological state of a subject having or suspected of having an HBV infection when compared to reference subjects known to be seropositive for HBV and to reference subjects that are seropositive for HCV. The discriminating peptides that distinguish the subjects that are seropositive for HBV from subjects that are seropositive for HCV comprise one or more sequence motifs that are enriched by greater than 100%, including the sequence motifs listed in FIG. 17A.

In some embodiments, the methods and systems herein identifies the serological state of a subject having or suspected of having an HBV infection when compared to reference subjects known to be seropositive for HBV and to reference subjects that are seropositive for WNV. The discriminating peptides that distinguish the subjects that are seropositive for HBV from subjects that are seropositive for WNV comprise sequence motifs that are enriched by greater than 100%, including the sequence motifs of FIG. 18A.

In some embodiments, the methods and systems herein identifies the serological state of a subject having or suspected of having an HCV infection when compared to reference subjects known to be seropositive for HCV and to reference subjects that are seropositive for WNV. The discriminating peptides that distinguish the subjects that are seropositive for HCV from subjects that are seropositive for WNV comprise sequence motifs that are enriched by greater than 100%, including the sequence motifs of FIG. 19A.

In another aspect, methods and systems are provided for determining the serological state of a subject having or being suspected of having one of a plurality of different infections selected from T. cruzi, HBV, HCV, and WNV, said method comprising: (a) contacting a sample from a subject suspected of having one of said infections to an array of peptides comprising at least 10,000 different peptides; (b) detecting the binding of antibodies present in said sample to at least 25 peptides on said array to obtain a combination of binding signals; (c) providing a first, a second, a third and at least a fourth set of differentiating binding signals for each of said plurality of infections, wherein each of said set differentiating binding signals distinguishes samples from a group of subjects being seropositive for one of said infections from a mixture of samples obtained from subjects each being seropositive for one of the remainder of said plurality of infections; (d) combining said sets of differentiating binding signals to obtain a multiclass set of differentiating binding signals, wherein said multiclass set differentiates each of said plurality of different infections from each other; and (e) comparing said combination of binding signals obtained in step (b) to said multiclass set of differentiating binding signals, thereby identifying the serological state of said subject. In some embodiments, the method further comprises identifying a set of discriminating peptides for each of said first, second, third, and at least fourth set of differentiating binding signals. In some embodiments, the first, second, third, and at least fourth set of discriminating peptides that distinguish a plurality of different infections selected from T. cruzi, HBV, HCV, and WNV, from each other further comprises differentiating peptides comprising sequence motifs that are enriched by greater than 100% selected from the list in FIG. 20A when compared to the at least 10,000 peptides in said array.

In some embodiments, the first set of discriminating peptides display signals that distinguish samples that are seropositive for T. cruzii from a mixture of samples that each are seropositive for one of HBV, HCV, and WNV. The discriminating peptides that distinguish samples that are seropositive for T. cruzii from a mixture of samples that each are seropositive for one of HBV, HCV, and WNV are enriched by greater than 100% in one or more sequence motifs listed in FIG. 10A, when compared to the at least 10,000 peptides in said array. In some embodiments, the second set of discriminating peptides display signals that distinguish samples that are seropositive for HBV from a mixture of samples that each are seropositive for one of T. cruzii, HCV, and WNV. The discriminating peptides that distinguish samples that are seropositive for HBV from a mixture of samples that each are seropositive for one of T. cruzi, HCV, and WNV comprise one or more sequence motifs that are enriched by greater than 100%, including the sequence motifs listed in FIG. 11A, when compared to the at least 10,000 peptides in said array. In some embodiments, the third set of discriminating peptides display signals that distinguish samples that are seropositive HCV from a mixture of samples that each are seropositive for one of HBV, T. cruzi and WNV. The discriminating peptides that distinguish samples that are seropositive for HCV from a mixture of samples that each are seropositive for one of HBV, T. cruzii and WNV comprise sequence motifs that are enriched by greater than 100%, including the sequence motifs listed in FIG. 12A, when compared to the at least 10,000 peptides in said array. In some embodiments, the at least fourth set of discriminating peptides distinguishes samples that are seropositive for WNV from a mixture of samples that each are seropositive for one of HBV, HCV, and T. cruzi. The discriminating peptides that distinguish samples that are seropositive for WNV from a mixture of samples that each are seropositive for one of HBV, HCV, and T. cruzi comprise sequence motifs that are enriched by greater than 100%, including the sequence motifs listed in FIG. 13A, when compared to the at least 10,000 peptides in said array.

The method performance of any of the methods provided is characterized by an area under the receiver operator characteristic (ROC) curve (AUC) equal or greater than 0.6. In other embodiments, the method performance is characterized by an area under the receiver operator characteristic (ROC) curve (AUC) ranging from 0.60 to 0.69, 0.70 to 0.79, 0.80 to 0.89, or 0.90 to 1.0.

In another aspect, a method is provided for identifying at least one candidate biomarker for an infectious disease in a subject, the method comprising: providing a peptide array and incubating a biological sample from said subject to the peptide array; identifying a set of discriminating peptides bound to antibodies in the biological sample from said subject, the set of discriminating peptides displaying binding signals capable of differentiating samples that are seropositive for said infectious disease from samples that are seronegative for said infectious disease; querying a proteome database with each of the peptides in the set of discriminating peptides; aligning each of the peptides in the set of discriminating peptides to one or more proteins in the proteome database of the pathogen causing said infectious disease; and obtaining a relevance score and ranking for each of the identified proteins from the proteome database; wherein each of the identified proteins is a candidate biomarker for the disease in the subject. In some embodiments, the method further comprises obtaining an overlap score, wherein said score corrects for the peptide composition of the peptide library. The method of identifying the discriminating peptides comprises: (i) detecting the binding of antibodies present in samples form a plurality of subjects being seropositive for said disease to an array of different peptides to obtain a first combination of binding signals; (ii) detecting the binding of antibodies to a same array of peptides, said antibodies being present in samples from two or more reference groups of subjects, each group being seronegative for said disease, to obtain a second combination of binding signals; (iii) comparing said first to said second combination of binding signals; and (iv) identifying said peptides on said array that are differentially bound by antibodies in samples from subjects having said disease and the antibodies in said samples from two or more reference groups of subjects, thereby identifying said discriminating peptides. In some embodiments, the number of discriminating peptides corresponds to at least a portion of the total number of peptides on said array. In some embodiments, the number of discriminating peptides corresponds to at least 0.00005%, at least 0.0001%, at least 0.0005%, at least 0.0001%, at least 0.001%, at least 0.003%, at least 0.005%, at least 0.01%, at least 0.05%, at least 0.1%, at least 0.5%, at least 1%, at least 0.5%, at least 1.5%, at least 2%, at least 3%, at least 4%, at least 5%, at least 10%, at least 25%, at least 50%, at least 75%, at least 80%, or at least 90% of the total number of peptides on the array.

In some embodiments, the method provided identifies at least one candidate biomarker for Chagas disease. In some embodiments, the at least one candidate protein biomarker is selected from the list provided in Table 2 and Table 8. In some embodiments, the at least one protein biomarker is identified from at least a portion of the discriminating peptides provided in FIGS. 21A-N, Table 6 and Table 7. In some embodiments, the at least one protein biomarker is identified from at least 0.00005%, at least 0.0001%, at least 0.0005%, at least 0.0001%, at least 0.001%, at least 0.003%, at least 0.005%, at least 0.01%, at least 0.05%, at least 0.1%, at least 0.5%, at least 1%, at least 0.5%, at least 1.5%, at least 2%, at least 3%, at least 4%, at least 5%, at least 10%, at least 25%, at least 50%, at least 75%, at least 80%, or at least 90% of the discriminating peptides provided in FIGS. 21A-N, Table 6 and Table 7.

Disclosed herein are methods and systems for identifying at least one candidate biomarker for Chagas disease in a subject, the method comprising: (a) providing a peptide array and incubating a biological sample from said subject to the peptide array; (b) identifying a set of discriminating peptides bound to antibodies in the biological sample from said subject, the set of discriminating peptides displaying binding signals capable of differentiating samples that are seropositive for said infectious disease from samples that are seronegative for Chagas disease; (c) querying a proteome database with each of the peptides in the set of discriminating peptides; (d) aligning each of the peptides in the set of discriminating peptides to one or more proteins in the proteome database of the pathogen causing Chagas disease; and (e) obtaining a relevance score and ranking for each of the identified proteins from the proteome database, wherein each of the identified proteins is a candidate biomarker for Chagas disease in the subject. In some instances, the methods and systems disclosed herein further comprises obtaining an overlap score, wherein said score corrects for the peptide composition of the peptide library. In yet other aspects, the discriminating peptides disclosed herein are identified as having p-values of less than 10⁷.

In yet other aspects, the step of identifying said set of discriminating peptides comprises: (i) detecting the binding of antibodies present in samples form a plurality of subjects being seropositive for said disease to an array of different peptides to obtain a first combination of binding signals; (ii) detecting the binding of antibodies to a same array of peptides, said antibodies being present in samples from two or more reference groups of subjects, each group being seronegative for said disease, to obtain a second combination of binding signals; (iii) comparing said first to said second combination of binding signals; and (iv) identifying said peptides on said array that are differentially bound by antibodies in samples from subjects having Chagas disease and the antibodies in said samples from two or more reference groups of subjects, thereby identifying said discriminating peptides. In still other aspects, the number of discriminating peptides corresponds to at least a portion of the total number of peptides on said array. In some instances, the at least one candidate protein biomarker is selected from the list provided in Table 6. In still other instances, the at least one protein biomarker is identified from at least a portion of the discriminating peptides provided in FIGS. 21A-N, Table 6 and Table 7. In yet other embodiments, the discriminating peptides comprise one or more sequence motifs listed in FIG. 9B and FIGS. 23A-23C that are enriched in discriminating peptides among all peptides that contain the motif compared to discriminating peptides among all array peptides by greater than 100%. In still other aspects, peptide arrays comprising peptides that include one or more motifs provided in FIG. 23 are also disclosed herein.

The methods and systems provided herein are applicable to subjects including human and non-human mammals. In some embodiments, the sample used in the methods is a blood sample, including whole blood, plasma, and serum fractions thereof. In some embodiments, the sample is a serum sample. In other embodiments, the sample is a plasma sample. In yet other embodiments, the sample is a dried blood sample.

In some embodiments, the arrays utilized to perform the methods and systems described herein comprise at least 5,000 different peptides. In some embodiments, the arrays utilized to perform the methods and systems described herein comprise at least 10,000 different peptides. In some embodiments, the arrays utilized to perform the methods and systems described herein comprise at least 50,000 different peptides. In other embodiments, the arrays utilized to perform the methods and systems described herein comprise at least 100,000 different peptides. In other embodiments, the arrays utilized to perform the methods and systems described herein comprise at least 300,000 different peptides. In other embodiments, the arrays utilized to perform the methods and systems described herein comprise at least 500,000 different peptides. In other embodiments, the arrays utilized to perform the methods and systems described herein comprise at least 1,000,000 different peptides. In other embodiments, the arrays utilized to perform the methods and systems described herein comprise at least 2,000,000 different peptides.

In other embodiments, the arrays utilized to perform the methods and systems described herein comprise at least 3,000,000 different peptides. The different peptides can be synthesized from less than 20 amino acids. In some embodiments, the different peptides on the peptide array are at least 5 amino acids in length. In other embodiments, the different peptides on the peptide array are between 5 and 13 amino acids in length. The peptides can be deposited on the array surface. In other embodiments, the peptides can be synthesized in situ.

Any of the methods provided have a reproducibility of classification characterized by an AUC>0.6. In some embodiments, the reproducibility of classification characterized by an AUC is ranges from 0.60 to 0.69, 0.70 to 0.79, 0.80 to 0.89, or 0.90 to 1.0.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIGS. 1A-1C shows a schematic depicting the binding of antibodies in blood to peptide array features (FIG. 1A), and the differential fluorescent signals reflecting differences between the binding of antibodies in a sample from a reference subject that is seronegative for Chagas disease (FIG. 1B) and the binding of antibodies in a sample from a subject that is seropositive for Chagas disease to a same array of peptides (FIG. 1C).

FIGS. 2A-2D shows bar graphs representing the binding of monoclonal antibody (mAb) standards (4C1 (FIG. 2A), p53Ab1 (FIG. 2B), p53Ab8 (FIG. 2C) and LnkB2 (FIG. 2D) to cognate epitope control features on the array. A standard set of monoclonal antibodies was applied to arrays at 2.0 nM in triplicate. For each monoclonal antibody, the mean log 10 RFI of the cognate control features was used to calculate the Z-score. Z-scores are plotted separately for each control feature with the individual monoclonals plotted as individual bars. Error bars represent the standard deviation of the individual control feature Z-scores. The known epitope for each mAb is provided above each bar graph.

FIG. 3 shows a Volcano plot visualizing a set of library peptides displaying antibody-binding signals that are significantly different between Chagas seropositive and Chagas seronegative subjects. A volcano plot is used to assess this discrimination as the joint distribution of t-test p-values versus log differences in signal intensity means (log of ratios). The density of the peptides at each plotted position is indicated by the heat scale. The 356 peptides above the green dashed white discriminate between positive and negative disease by immunosignature technology (IST) with 95% confidence after applying a Bonferroni adjustment for multiplicity. The colored circles indicate individual peptides with intensities that are significantly correlated to the T. cruzi ELISA-derived signal over cutoff (S/CO) value either by a Bonferroni threshold of p<4e-7 (green) or a false discovery rate of <10% (blue). Most of the S/CO correlated peptides lie above the IST Bonferroni white dashed line.

FIGS. 4A and 4B show performance of immunosignature assay (IST) in distinguishing Chagas seropositive from seronegative donors. (FIG. 4A) Receiver Operating Characteristic (ROC) curve for the 2015 training cohort. The blue curve was generated by calculating the median of out-of-bag predictions in 100 four-fold cross-validation trials. (FIG. 4B) ROC curve for the 2016 verification cohort. The blue curve was generated by applying the training set-derived algorithm to predict the 2016 samples. Confidence intervals (CI), shown in gray, were estimated by bootstrap resampling of the donors in the training cohort, and estimated by the DeLong method (DeLong E R, et al. Biometrics 44:837-845 [1988]) in the verification cohort.

FIG. 5 shows signal intensity patterns displayed by the Chagas-classifying versus donor S/CO value. Heatmap ordering the ranges of signal intensities of the 370 library peptides that distinguish Chagas seropositive from Chagas-negative donors, with a side-bar graph relating these to each donor's ELISA S/CO value.

FIG. 6 shows a histogram of the alignment scores from the top 370 peptides against all Chagas proteins (depicted in the blue bars). The mapping algorithm was repeated with 10 equivalent alignments of 370 randomly chosen library peptides. Each yielded histograms that are shown as rainbow-colored line plots.

FIG. 7 shows the representation of the levels of similarity of library classifying peptides to a family of T. cruzi protein-antigens. Alignment of the top 370 peptides to the mucin II GPI-attachment site is represented as a bar chart in which the bars have been replaced by the amino acid composition at each alignment position, using the standard single-letter code. The x-axis indicates the conserved amino acid at the aligned position in mucin II proteins. The y-axis represents coverage of that amino acid position by the classifying peptides. The height of all letters at a position is the absolute number alignments at each position, where the percent of each letter-bar taken up by a single amino acid equals the percent composition of alignments at that position.

FIG. 8 shows the probabilities of Chagas, Hepatitis B, Hepatitis C and West Nile Virus class assignments. Mean predicted probabilities for each sample were calculated by out-of-bag predictions from four-fold cross-validation analyses using a multiclass SVM machine classifier, iterated 100 times. Each sample has a predicted class membership for each disease class ranging from 0 (black) to 100% (white).

FIGS. 9A-9F show the amino acids (A) and motifs (B-F) that are enriched in the top discriminating peptides that distinguish samples of seropositive subjects infected with Chagas from sample from subjects that are seronegative (healthy) for Chagas.

FIGS. 10A and 10B show the motifs (A) and amino acids (B) that are enriched in the top discriminating peptides that distinguish samples of subjects infected with Chagas from sample from a group of subjects infected with HBV, HCV, and WNV.

FIGS. 11A and 11B show the motifs (A) and amino acids (B) that are enriched in the top discriminating peptides that distinguish samples of subjects infected with HBV from sample from a group of subjects infected with Chagas, HCV, and WNV.

FIGS. 12A and 12B show the motifs (A) and amino acids (B) that are enriched in the top discriminating peptides that distinguish samples of subjects infected with HCV from sample from a group of subjects infected with HBV, Chagas, and WNV.

FIGS. 13A and 13B show the motifs (A) and amino acids (B) that are enriched in the top discriminating peptides that distinguish samples of subjects infected with WNV from sample from a group of subjects infected with HBV, HCV, and Chagas.

FIGS. 14A and 14B show the motifs (A) and amino acids (B) that are enriched in the top discriminating peptides that distinguish samples of subjects infected with Chagas from samples from subjects infected with HBV.

FIGS. 15A and 15B show the motifs (A) and amino acids (B) that are enriched in the top discriminating peptides that distinguish samples of subjects infected with Chagas from samples from subjects infected with HCV.

FIGS. 16A and 16B show the motifs (A) and amino acids (B) that are enriched in the top discriminating peptides that distinguish samples of subjects infected with Chagas from samples from subjects infected with WNV.

FIGS. 17A and 17B show the motifs (A) and amino acids (B) that are enriched in the top discriminating peptides that distinguish samples of subjects infected with HBV from samples from subjects infected with HCV.

FIGS. 18A and 18B show the motifs (A) and amino acids (B) that are enriched in the top discriminating peptides that distinguish samples of subjects infected with HBV from samples from subjects infected with WNV.

FIGS. 19A and 19B show the motifs (A) and amino acids (B) that are enriched in the top discriminating peptides that distinguish samples of subjects infected with HCV from samples from subjects infected with WNV.

FIGS. 20A and 20B show the motifs (A) and amino acids (B) that are enriched in the top discriminating peptides that distinguish samples from subjects infected with Chagas, HCV, HBV, and WNV from each other determined by a multiclass classifier.

FIGS. 21A-21N show the sequences of the discriminating peptides that distinguish seropositive Chagas samples from seronegative Chagas samples.

FIG. 22 shows a Volcano plot visualizing a set of library peptides from V16, V13 and IEDB libraries (V16 array) displaying antibody-binding signals that are significantly different between Chagas seropositive and Chagas seronegative subjects.

FIG. 23A-23C shows exemplary motifs that were found to be enriched in the peptides in the V16 array that distinguish seropositive Chagas samples from seronegative Chagas samples.

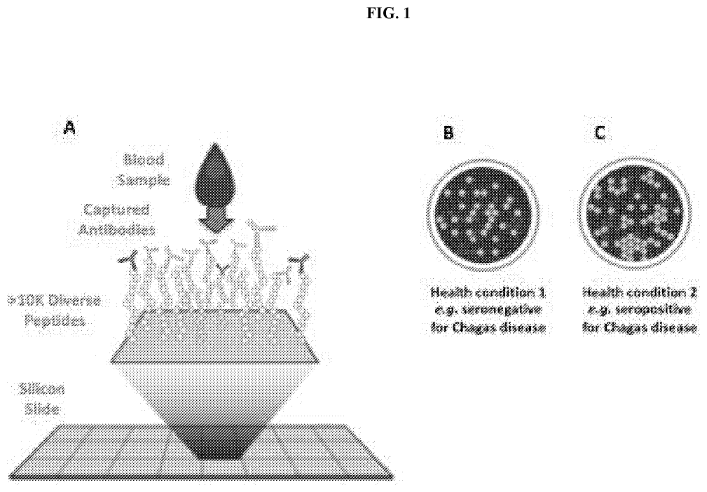

### DETAILED DESCRIPTION OF THE INVENTION

The disclosed embodiments concern methods, apparatus, and systems for identifying an infection in a subject. Additionally, the methods, apparatus, and systems are provided for identifying candidate biomarkers, including protein biomarkers useful for the diagnosis, prognosis, monitoring and screening of infections, and/or as a therapeutic target for treatment of an infection.

The identification of any one infection and of the candidate biomarkers for the infection is founded on the presence of an immunosignature assay (IST), which exhibit the binding of antibodies from a subject to a library of peptides on an array as a pattern of binding signals i.e. a combination of binding signals, that reflect the immune status of the subject. IST is a combination of discriminating peptides that differentially bind antibodies present in a sample of a subject relative to a combination of peptides that are bound by antibodies present in reference samples. The patterns of binding signals comprise binding information that can be indicative of a state e.g. seropositive or seronegative, of a symptomatic, and/or of an asymptomatic state consequent to an infection.

The methods described herein provide several advantages over existing methods. In one aspect, the methods described can detect infections in both symptomatic and asymptomatic subjects. The methods are highly efficient in that a single testing event i.e. a single microarray signature can assess for the presence of any one of a plurality of infections, and the diagnosis of multiple infections can be determined simultaneously. The identification of any one infection is only limited by the number of different infections for which discriminating peptides have been identified. The methods, apparatus, and systems described herein are suitable for identifying infections caused by a wide variety of pathogens including bacteria, viruses, fungi, protozoans, worms, and infestations, and have applications in the fields of research, medical and veterinary diagnostics, and health surveillance, such as tracking the spread of an outbreak caused by a pathogen.

Methods, apparatus and systems are provided herein that enable detection and diagnosis of infections using a single noninvasive screening method that identifies differential patterns of peripheral-blood antibody binding to peptide arrays. Differential binding of patient samples to peptide arrays results in specific binding patterns, i.e., immunosignature assay (IST) results that are indicative of the health condition, e.g. infection, of the patient. Additionally, the apparatus and systems provided herein allow for the identification of antigens or binding partners to antibodies of the biological sample, which can be assessed as candidate biomarkers for targeted therapeutic interventions.

Typically, an immunosignature characteristic of a condition is determined relative to one or more reference immunosignatures, which are obtained from one or more different sets of reference samples, each set being obtained from one or more groups of reference subjects, each group having a different condition e.g. a different infection. For example, an immunosignature obtained from a test subject identifies the infection of the test subject when compared to immunosignatures of reference subjects without infection and/or with different infections induced by different pathogens. Accordingly, comparison of immunosignatures from a test subject with those of reference subjects can determine the condition e.g. infection, of the test subject. A reference group can be a group of healthy subjects, and the condition is referred to herein as a healthy condition. Healthy subjects are typically those who do not have the infection that is being tested, or known to be seronegative for the infection that is being tested.

The methods provided can detect a number of different infections in samples e.g. blood, from different individuals within a population of symptomatic or asymptomatic subjects that are seropositive for the different infections with high performance, sensitivity and specificity. The infections that can be detected according to the methods provided include without limitation infections caused by microorganisms, including bacteria, viruses, fungi, protozoans, parasitic organisms and worms.

In some embodiments, the IST is based on diverse yet reproducible patterns of antibody binding to an array of peptides that are selected to provide an unbiased sampling of at least a portion of amino acid combinations less than 20 amino acids rather than represent known proteomic sequences. A peptide bound by an antibody in a sample from a subject may not be the natural target sequence, but may instead mimic the sequence or structure of the cognate natural epitope. For example, none of the peptides in the IST library described in Example 1 are identical matches to any 9 mer sequence in known proteome databases. This is not surprising since the number of possible 9 mer peptide sequences is several orders of magnitude greater than the number of contiguous 9 mer sequences in the proteome databases. Accordingly, the probability of any mimetic-peptide corresponding exactly to a natural sequence is low. Each IST peptide sequence that is selectively bound by an antibody could be a functional surrogate of the epitope that the antibody recognized in vivo. Consequently, the sequences of proteins comprising part or all of the antibody-bound array peptide sequence can serve to identify candidate protein biomarkers, which can be assessed as therapeutic targets.

In one aspect, a method is provided for identifying the serological state of a subject having or suspected of having at least one infection comprising: (a) contacting a sample from the subject to an array of peptides comprising at least 10,000 different peptides; (b) detecting the binding of antibodies present in the sample to at least 25 peptides on the array to obtain a combination of binding signals; and (c) comparing the combination of binding signals of the sample from the subject to one or more groups of combinations of reference binding signals, wherein at least one of each of the groups of combinations of reference binding signals are obtained from a plurality of reference subjects known to be seropositive for an infection, and wherein at least one of each of the groups of combinations of reference binding signals are obtained from a plurality of subjects known to be seronegative for an infection, thereby determining the serological state of the subject. In some embodiments, reference subjects that are seronegative for one infection can be seropositive for a different infection. The array peptides can be deposited or can be synthesized in situ on a solid surface. In some embodiments, the method performance can be characterized by an area under the receiver operator characteristic (ROC) curve (AUC) being greater than 0.6. In some embodiments, the reproducibility of classification from an AUC ranges from 0.60 to 0.69, 0.70 to 0.79, 0.80 to 0.89, or 0.90 to 1.0.

In some embodiments, the method further comprises identifying a combination of differentiating reference binding signals that distinguish samples from reference subjects known to be seropositive for the infection from samples from reference subjects known to be seronegative for the same infection, and identifying the combination of the array peptides that display the combination of differentiating binding signals. The combination of differentiating binding signals can comprise signals that are increased or decreased, newly added signals, and/or signals that are lost in the presence of an infection relative to the corresponding binding signals obtained from reference samples. The array peptides that display the combination of differentiating binding signals are known as discriminating peptides. The term “discriminating” when used in reference to array peptides is used herein interchangeably with “classifying”. In some embodiments, a combination of differentiating reference binding signals comprises a combination of binding signals to at least 1, at least 2, at least 5, at least 10, at least 15, at least 20, at least 25, at least 30, at least 35, at least 40, at least 45, at least 50, at least 60, at least 70, at least 80, at least 90, at least 100, at least 125, at least 150, at least 175, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10000, at least 20000, or more discriminating peptides on an array. For example, at least 25 peptides on an array of 10,000 peptides are identified as discriminating peptides for a given condition. In some embodiments, each combination of differentiating binding signals is obtained by detecting the binding of antibodies present in a reference sample from each of a plurality of reference subjects to at least 25 peptides on same arrays of peptides comprising at least 10,000 different peptides. In some embodiments, the peptides are synthesized in situ. In some embodiments, discriminating peptides are identified from antibodies binding differentially to peptide arrays comprising a library of at least 5,000, at least 10,000, at least 15,000, at least 20,000, at least 25,000, at least 50,000, at least 100,000, at least 200,000, at least 300,000, at least 400,000, at least 500,00, at least 1,000,000, at least 2,000,000, at least 3,000,000, at least 4,000,000, at least 5,000,000 or at least 100,000,000 or more different peptides on the array substrate. In some embodiments, the differential binding signal is

In some embodiments, at least 0.00005%, at least 0.0001%, at least 0.0005%, at least 0.0001%, at least 0.001%, at least 0.003%, at least 0.005%, at least 0.01%, at least 0.05%, at least 0.1%, at least 0.5%, at least 1%, at least 0.5%, at least 1.5%, at least 2%, at least 3%, at least 4%, at least 5%, at least 10%, at least 25%, at least 50%, at least 75%, at least 80%, or at least 90%, of the total number of peptides on an array are discriminating peptides. In other embodiments, all of the peptides on an array are discriminating peptides.

Binding Assay

The immunosignature of a subject is identified as a pattern of binding of antibodies that are bound to the array peptides. The peptide array can be contacted with a sample e.g. blood, plasma or serum, under any suitable conditions to promote binding of antibodies in the sample to peptides immobilized on the array. Thus, the methods of the invention are not limited by any specific type of binding conditions employed. Such conditions will vary depending on the array being used, the type of substrate, the density of the peptides arrayed on the substrate, desired stringency of the binding interaction, and nature of the competing materials in the binding solution. In a preferred embodiment, the conditions comprise a step to remove unbound antibodies from the addressable array. Determining the need for such a step, and appropriate conditions for such a step, are well within the level of skill in the art.

Any suitable detection technique can be used in the methods and systems described herein for detecting binding of antibodies in a sample to peptides on the array to generate an immune profile consequent to an infection. In one embodiment, any type of detectable label can be used to label peptides on the array, including but not limited to radioisotope labels, fluorescent labels, luminescent labels, and electrochemical labels (i.e.: ligand labels with different electrode mid-point potential, where detection comprises detecting electric potential of the label). Alternatively, bound antibodies can be detected, for example, using a detectably labeled secondary antibody.

Detection of signal from detectable labels is well within the level of skill in the art. For example, fluorescent array readers are well known in the art, as are instruments to record electric potentials on a substrate (For electrochemical detection see, for example, J. Wang (2000) Analytical Electrochemistry, Vol., 2nd ed., Wiley—VCH, New York). Binding interactions can also be detected using other label-free methods such a s SPR and mass spectrometry. SPR can provide a measure if dissociation constants and dissociation rates. The A-100 Biocore/GE instrument, for example, is suitable for this type of analysis. FLEX chips can be used to up to 400 binding reactions on the same support.

Alternatively, binding interactions between antibodies in a sample and the peptides on an array can be detected in a competition format. A difference in the binding profile of an array to a sample in the presence versus absence of a competitive inhibitor of binding can be useful in characterizing the sample.

### Classification Algorithms

Analyses of the antibody binding signal data i.e. immunosignature data (IST), and the diagnosis derived therefrom are typically performed using various algorithms and programs. The antibody binding pattern produced by the labeled secondary antibody bound to primary antibodies is scanned using, for example, a laser scanner. The images of the binding signals acquired by the scanner can be imported and processed using software such as the GenePix Pro 8 software (Molecular Devices, Santa Clara, Calif.), to provide tabular information for each peptide, for example, in a continuous value ranging from 0-65,535. Tabular data can be imported and statistical analysis performed using, for example, into the R language and environment for statistical computing (R Foundation for Statistical Computing, Vienna, Austria. URL https://www.R-project.org/).

Peptides displaying differential signaling patterns, i.e. discriminating peptides, between samples obtained from reference subjects with different conditions e.g. seropositive subjects consequent to an infection, can be identified using known statistical tests such as a Student's T-test or ANOVA. The statistical analyses are applied to select the discriminating peptides that distinguish the different conditions at predetermined stringency levels. In some embodiments, a list of the most discriminating peptides can be obtained by ranking the peptides by statistical means such as their p-value. For example, discriminating peptides can be ranked and identified as having p-values of between zero and one. The cutoff for the p-value can be further adjusted to account for instances when several dependent or independent statistical tests are being performed simultaneously on a single data set. For example, a Bonferroni correction can be used to reduce the chances of obtaining false positives when multiple pairwise tests are performed on a single set of data. The correction is dependent on the size of the array library. In some embodiments, the cutoff p-value for determining the discriminating can be adjusted to less than 10⁻²⁰, less than 10⁻¹⁹, less than 10⁻¹⁸, less than 10⁻¹⁷, less than 10⁻¹⁶, less than 10⁻¹⁵, less than 10⁻¹⁴, less than 10⁻¹³, less than 10⁻¹², less than 10⁻¹¹, less than 10⁻¹⁰, less than 10⁻⁹, less than 10⁻⁸, less than 10⁻⁷, less than 10⁻⁶, or less than 10⁻⁵, or less than 10⁻⁴, or less than 10⁻³, or less than 10⁻². The adjustment is dependent on the size of the array library. Alternatively, discriminating peptides are not ranked, and the binding signal information displayed up to all of the identified discriminating peptides is used to classify a condition e.g. the serological state of a sample.

Subsequently, binding signal information of the discriminating peptides selected following statistical analysis can be subsequently imported into a machine learning algorithm to obtain a statistical or mathematical model i.e. a classifier, that classifies the antibody profile data with accuracy, sensitivity and specificity, and determines the serological state of a sample, and other applications described elsewhere herein. Any one of the many computational algorithms can be utilized for the classification purposes.

The classifiers can be rule-based or can be computationally intelligent. Further, the computationally intelligent classification algorithms can be supervised or unsupervised. A basic classification algorithm, Linear Discriminant Analysis (LDA) may be used in analyzing biomedical data in order to classify two or more disease classes. LDA can be, for example, a classification algorithm. A more complex classification method, Support Vector Machines (SVM), uses mathematical kernels to project the original predictors to higher-dimensional spaces, then identifies the hyperplane that optimally separates the samples according to their class. Some common kernels include linear, polynomial, sigmoid or radial basis functions. A comparative study of common classifiers described in the art is described in (Kukreja et al, BMC Bioinformatics. 2012; 13: 139). Other algorithms for data analysis and predictive modeling based on data of antibody binding profiles include but are not limited to Naive Bayes Classifiers, Logistic Regression, Quadratic Discriminant Analysis, K-Nearest Neighbors (KNN), K Star, Attribute Selected Classifier (ACS), Classification via clustering, Classification via Regression, Hyper Pipes, Voting Feature Interval Classifier, Decision Trees, Random Forest, and Neural Networks, including Deep Learning approaches.

In some embodiments, antibody binding profiles are obtained from a training set of samples, which are used to identify the most discriminative combination of peptides by applying an elimination algorithm based on SVM analysis. The accuracy of the algorithm using various numbers of input peptides ranked by level of statistical significance can be determined by cross-validation. To generate and evaluate antibody binding profiles of a feasible number of discriminating peptides, multiple models can be built, using a plurality of discriminating peptides to identify the best performing model. While the method does not exclude limiting the number of peptides, the method can exploit all or substantially all available peptide binding information e.g. binding signals. Thus, the method contrasts with approaches that attempt to determine a priori the peptides whose sequences can be utilized for binding purposes. In some embodiments, up to all of the peptides on the array are discriminating peptides. In some embodiments, at least 25, at least 50, at least 75, at least 100, at least 200, at least 300, at least 400, at least 500, at least 750, at least 1000, at least 1500, at least 2000, at least 3000, at least 4000, at least 5000, at least 6000, at least 7000, at least 8000, at least 9000, at least 10,000, at least 11,000 at least 12,000 at least 13,000 at least 14,000 at least 15,000 at least 16,000 at least 17,000 at least 18,000 at least 19,000 at least 20,000 or more discriminating peptides are used to train a specific disease-classifying model. In some embodiments at least 0.00001%, at least 0.0001%, at least 0.0005%, at least 0.001%, at least 0.005%, at least 0.01%, at least 0.05%, at least 0.1%, at least 0.5%, at least 1.0%, at least 2%, at least 3%, at least 4%, at least 5%, at least 10%, at least 20%, at least 30%, at least 40%, at least 50%, at least 60%, at least 70%, at least 80%, at least 90%, at least 95%, or at least 99% of the total number of peptides on the array are discriminating peptides, and the corresponding binding signal information is used to train a specific condition-classifying model. In some embodiments, the signal information obtained for all of the peptides on the array is used to train the condition-specific model.

Multiple models comprising different numbers of discriminating peptides can be generated, and the performance of each model can be evaluated by a cross-validation process. An SVM classifier can be trained and cross-validated by assigning each sample of a training set of samples to one of a plurality of cross-validation groups. For example, for a four-fold cross-validation, each sample is assigned to one of four cross-validation groups such that each group comprises test and control i.e. reference samples; one of the cross-validation groups e.g. group 1, is held-out, and an SVM classifier model is trained using the samples in groups 2-4. Peptides that discriminate test cases and reference samples in the training group are analyzed and ranked, for example by statistical p-value; the top k peptides are then used as predictors for the SVM model. To elucidate the relationship between the number of input predictors and model performance, and to guard against overfitting, the sub=loop is repeated for a range of k, e.g. 25, 50, 100, 250, 1000, 200, 3000 top peptides or more. Predictions i.e. classification of samples in group 1 are made using the model generated using groups 2-4. Models for each of the four groups are generated, and the performance (AUC, sensitivity and/or specificity) is calculated using all the predictions from the 4 models using signal binding data from true disease samples. The cross-validation steps are repeated at least 100 times, and the average performance is calculated relative to a confidence interval e.g. 95%. Diagnostic visualization can be generated using e.g. model performance relative to the number of input peptides.

An optimal model/classifier based on antibody binding information to a set of discriminating input peptides (list of the most discriminating peptides, k) is selected and used to predict the disease status of a test set. The performance of different classifiers is determined using a validation set, and using a test set of samples, performance characteristics such as accuracy, sensitivity, specificity, and Area Under the Curve (AUC) of the Receiver Operating Characteristic (AUC) curve are obtained from the model having the greatest performance. In some embodiments, different sets of discriminating peptides are identified to distinguish different conditions. Accordingly, an optimal model/classifier based on a set of the most discriminating input peptides is established for each of the health conditions e.g. infections, to be identified in different subjects.

### Classification of Conditions

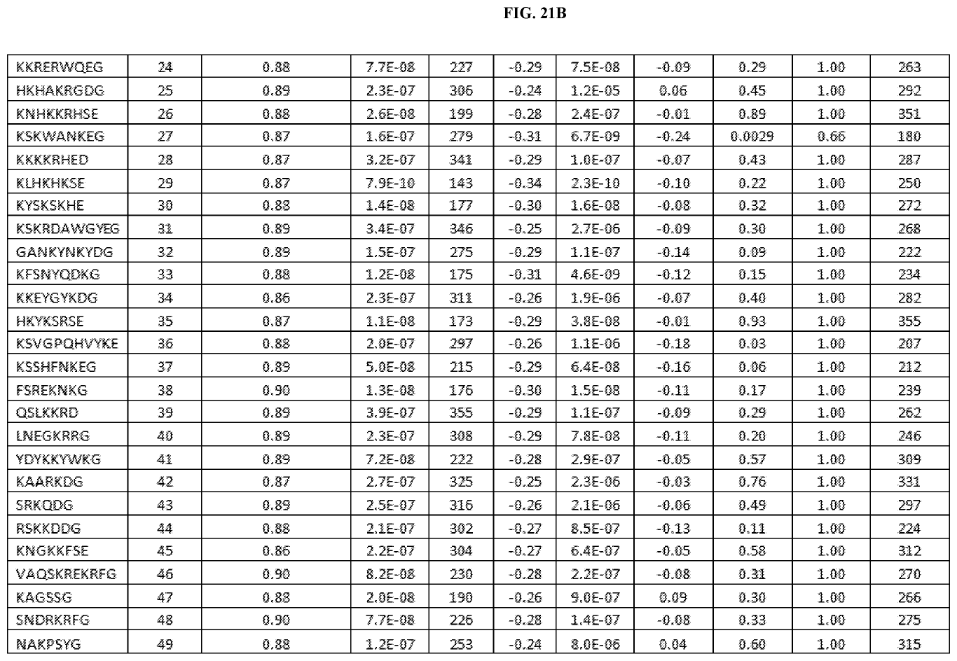

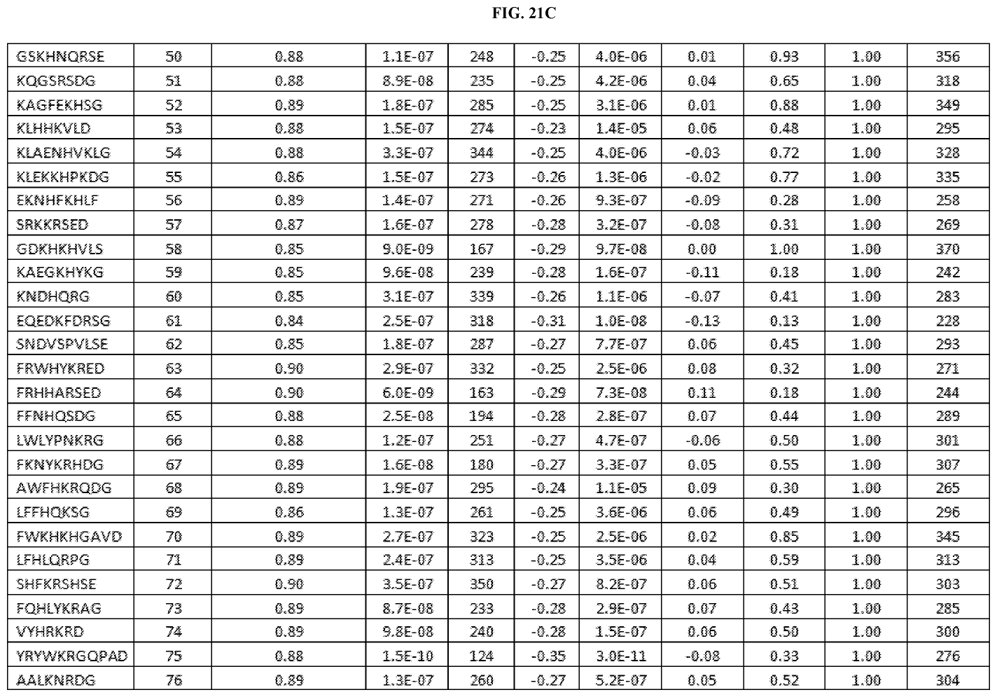

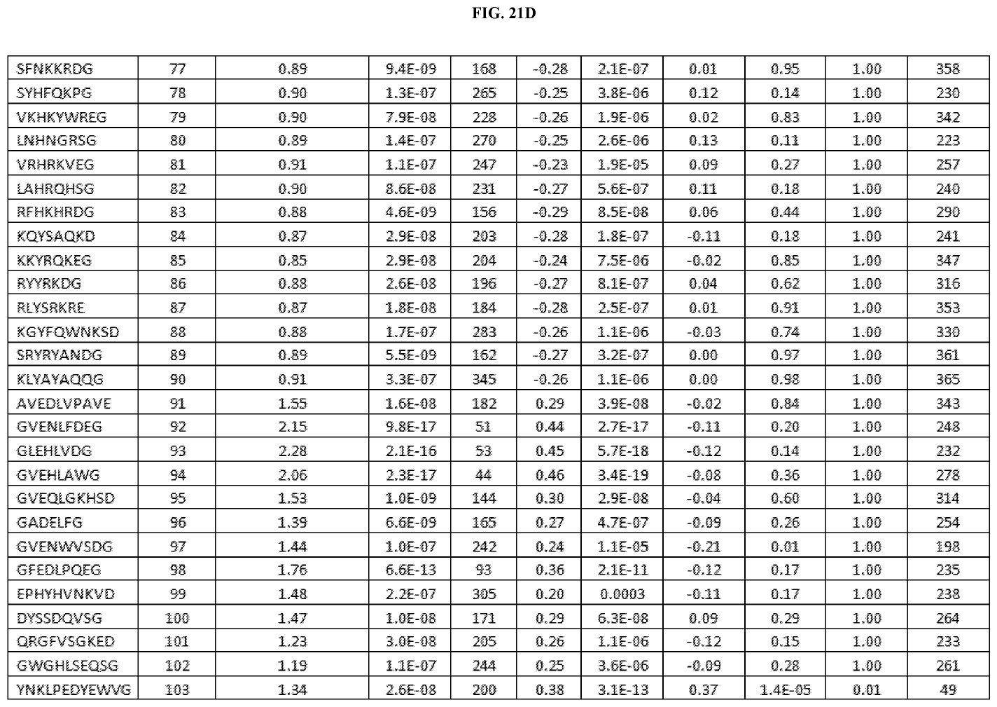

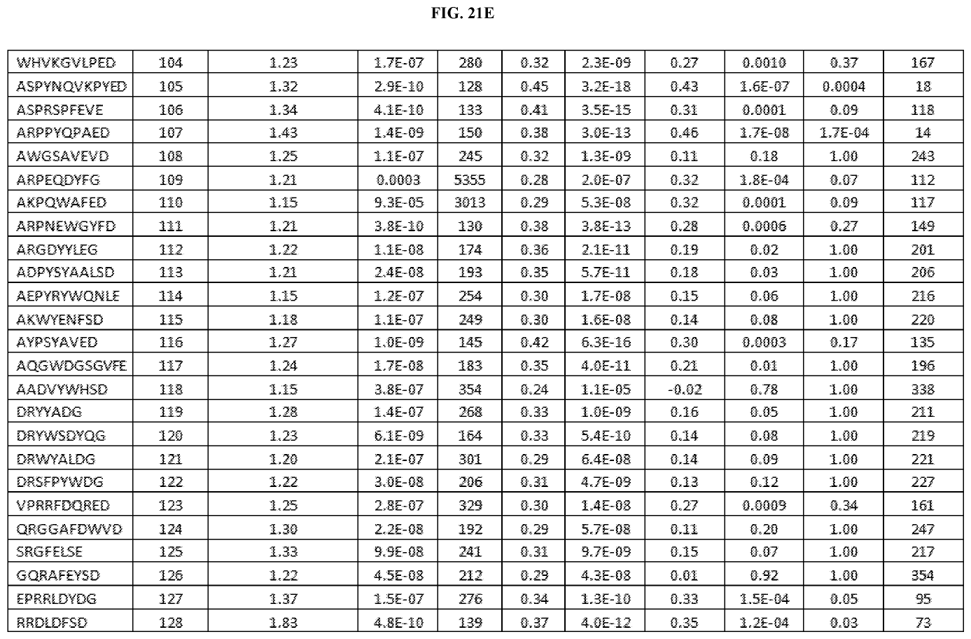

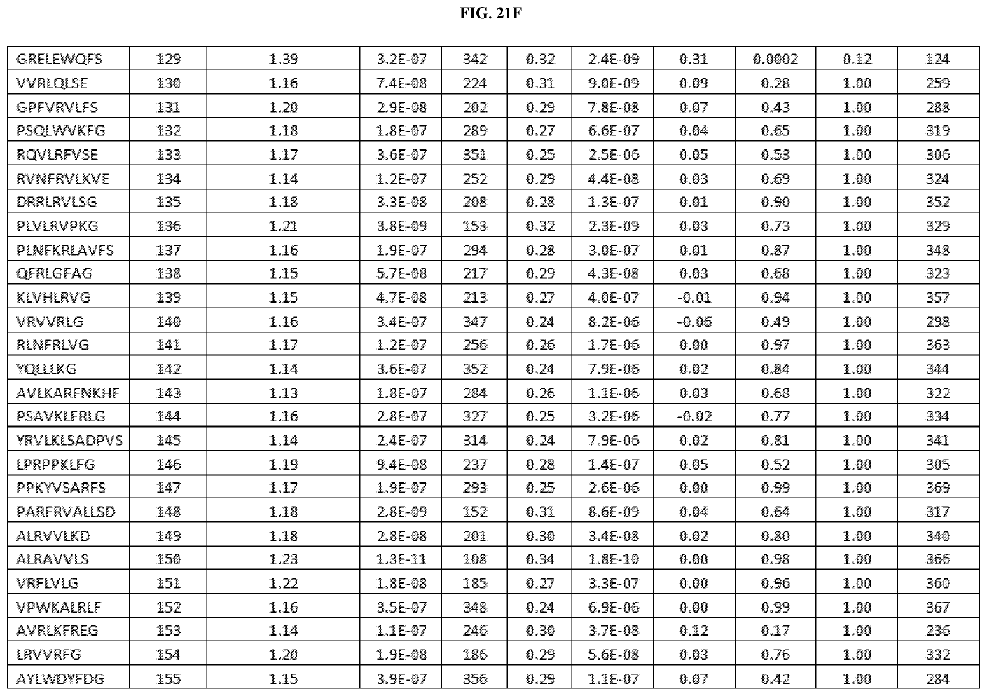

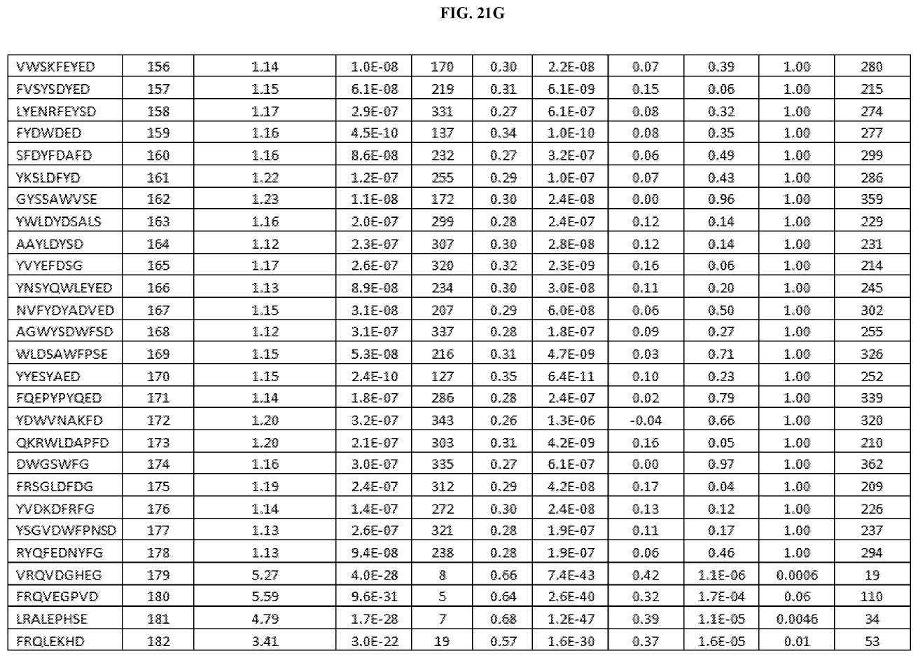

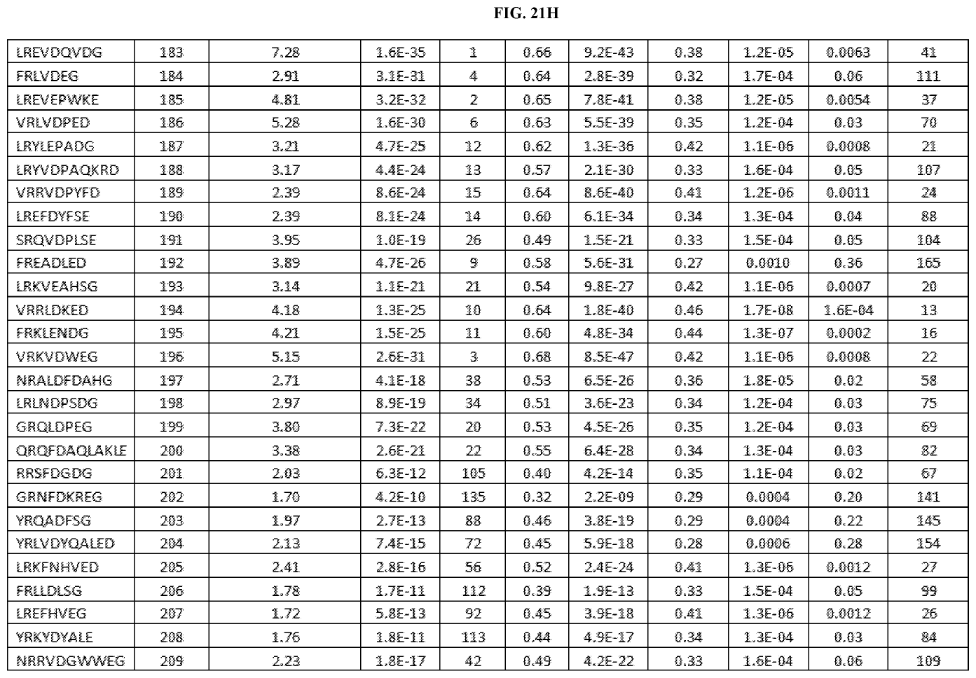

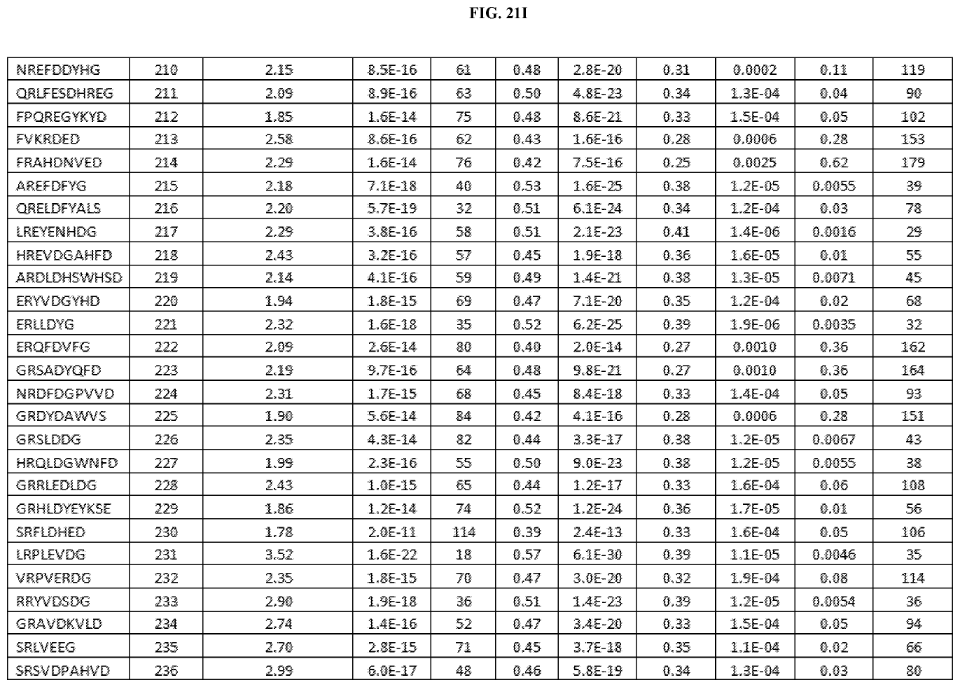

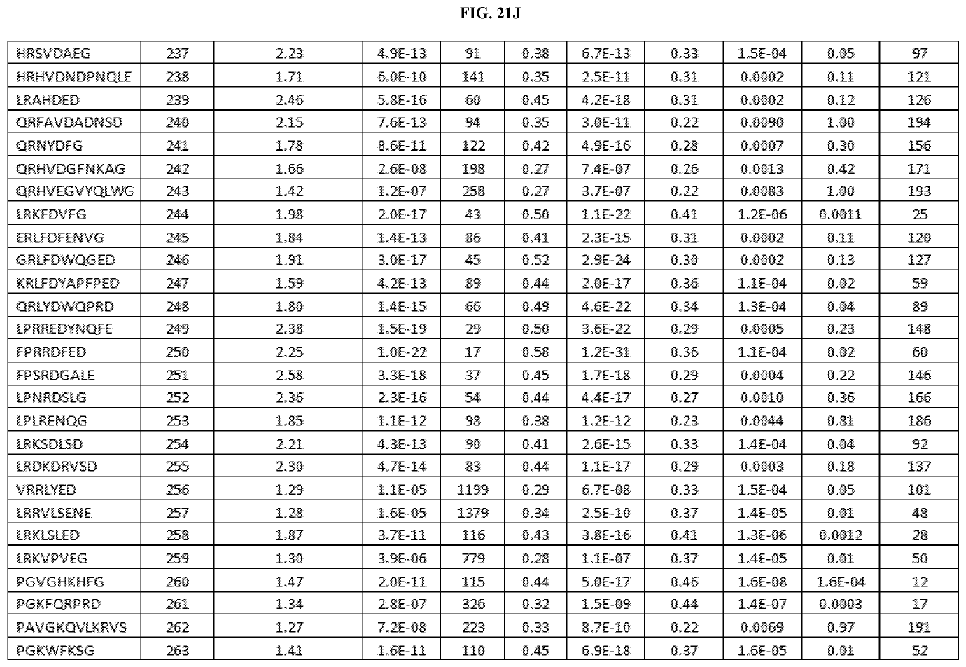

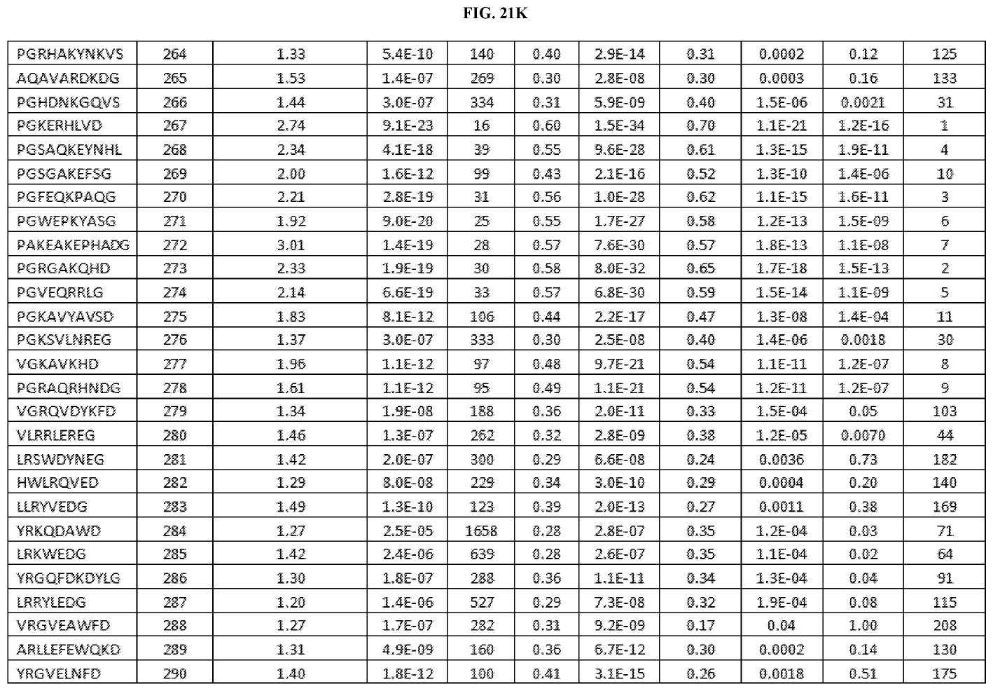

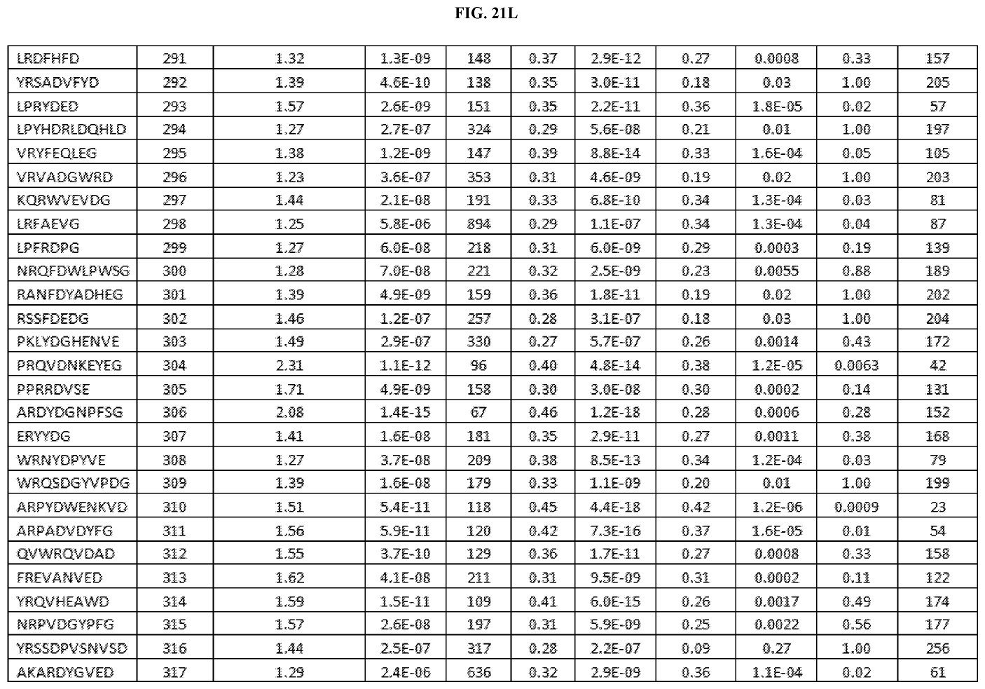

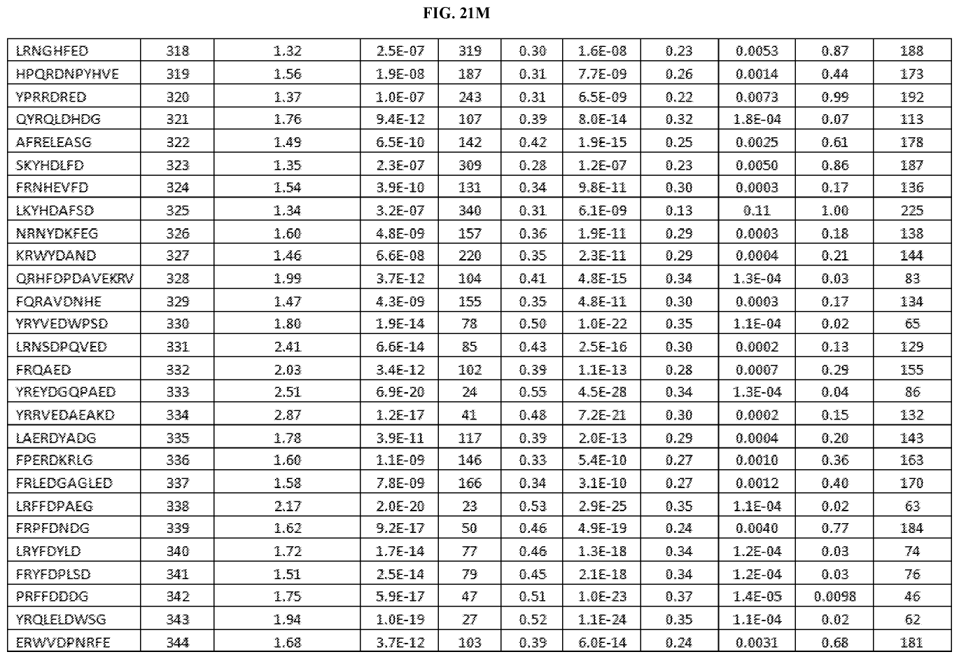

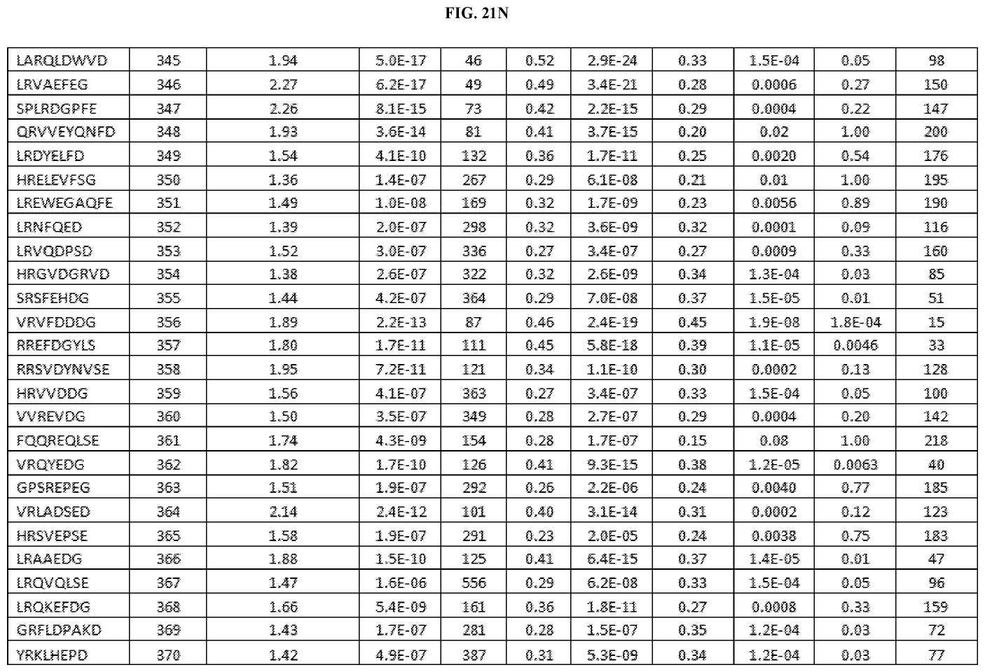

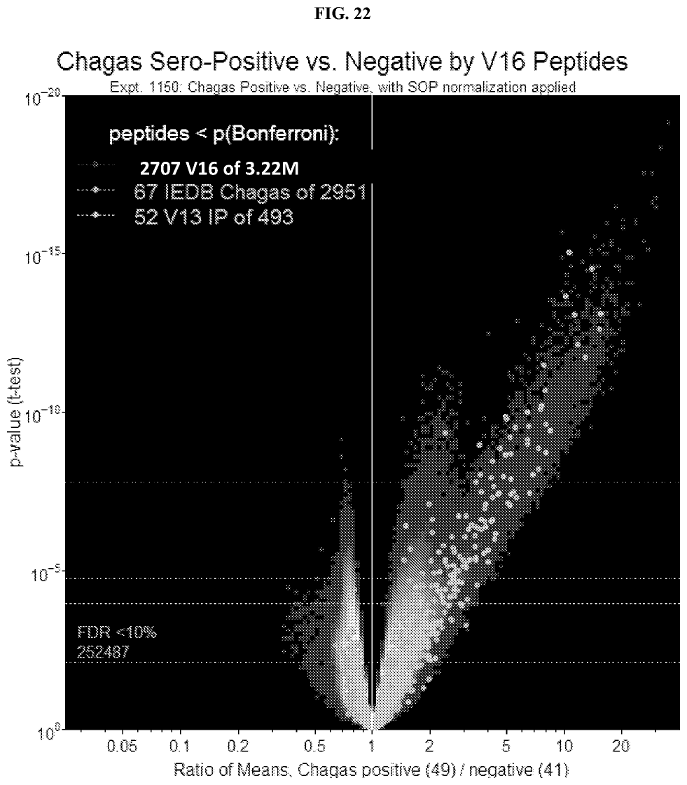

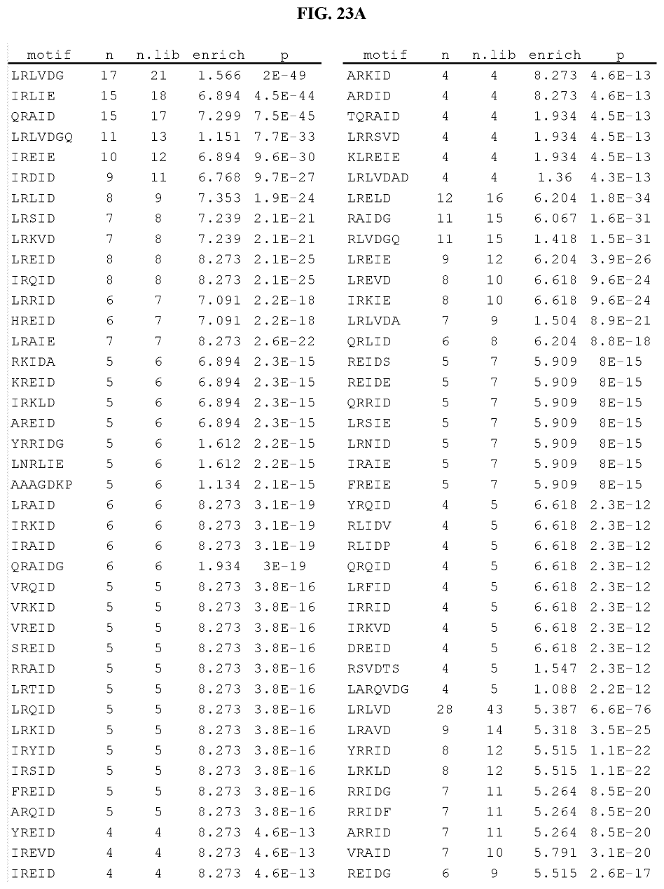

In some embodiments, individual binary classifiers can be obtained to identify the serological state of an infection relative to the serological state of a reference condition e.g. a single different infection, and a combination of discriminating peptides utilized by the classifier is provided. For example, as shown in Example 3, an optimal classifier based on a combination of discriminating peptides is selected to predict the serological state of a subject having or suspected of having a T. cruzi infection. In example 3, the discriminating peptides were determined to distinguish samples from subjects that were seropositive with a T. cruzi infection from reference samples from a group of subjects who were seronegative for T. cruzi (FIGS. 21A-N).

The characteristics of the combination of the discriminating peptides include the prevalence of one or more amino acids, and/or the prevalence of specific sequence motifs present in the identified discriminating peptides. Enrichment of amino acid and motif content is relative to the corresponding total amino acid and motif content of all the peptides in the array library. In some embodiments, the discriminating peptides of the immunosignature binding patterns that distinguish a subject that is seropositive consequent to an infection from reference subjects that are seronegative for the same infection can be enriched in at least one, at least two, at least three, at least four, at least five, at least six, at least seven, at least eight, at least nine, or at least ten different amino acids. In some embodiments, enrichment of the amino acids in discriminating peptides can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% relative to the total content of each of the amino acids present in all the library peptides.

Similarly, in some embodiments, the discriminating peptides of the immunosignature binding patterns that distinguish a subject that is seropositive consequent to an infection from reference subjects that are seronegative for the same infection can be enriched in at least one, at least two, at least three, at least four, at least five, at least six, at least seven, at least eight, at least nine, or at least ten different sequence motifs. Enrichment of the sequence motifs can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% in at least one motif relative to the total content of each of the motifs present in all library peptides.

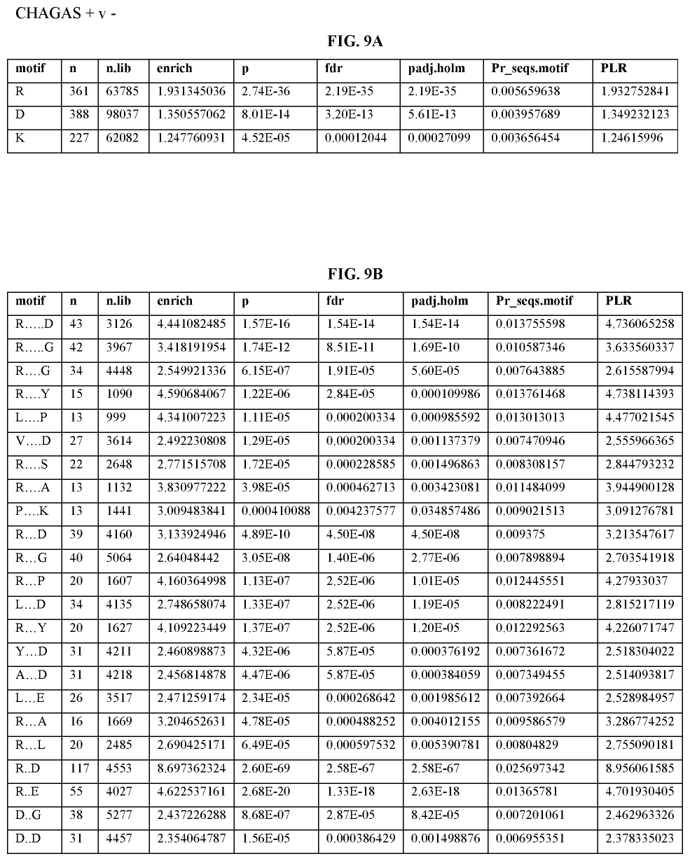

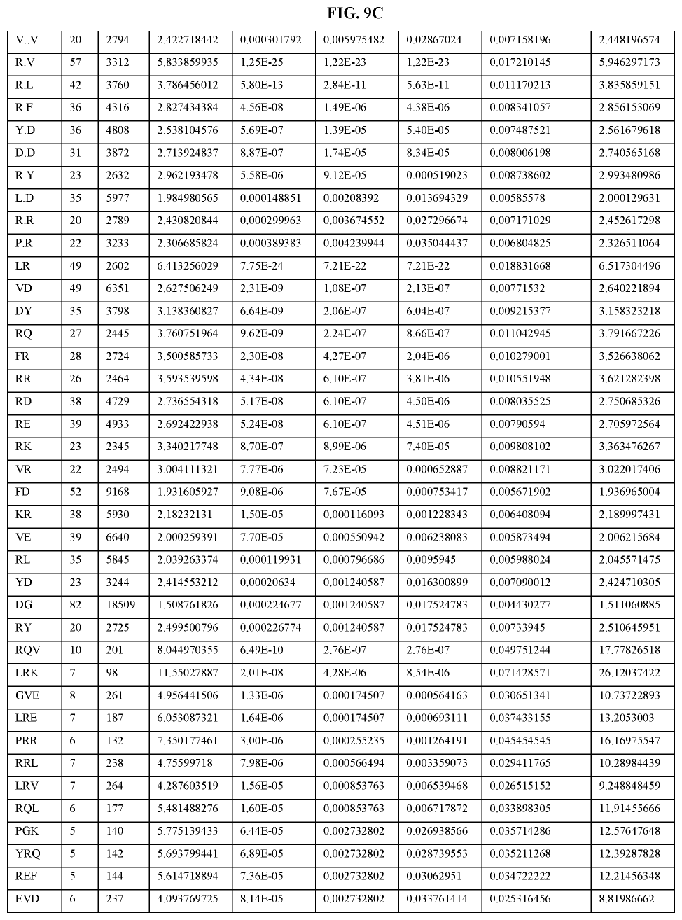

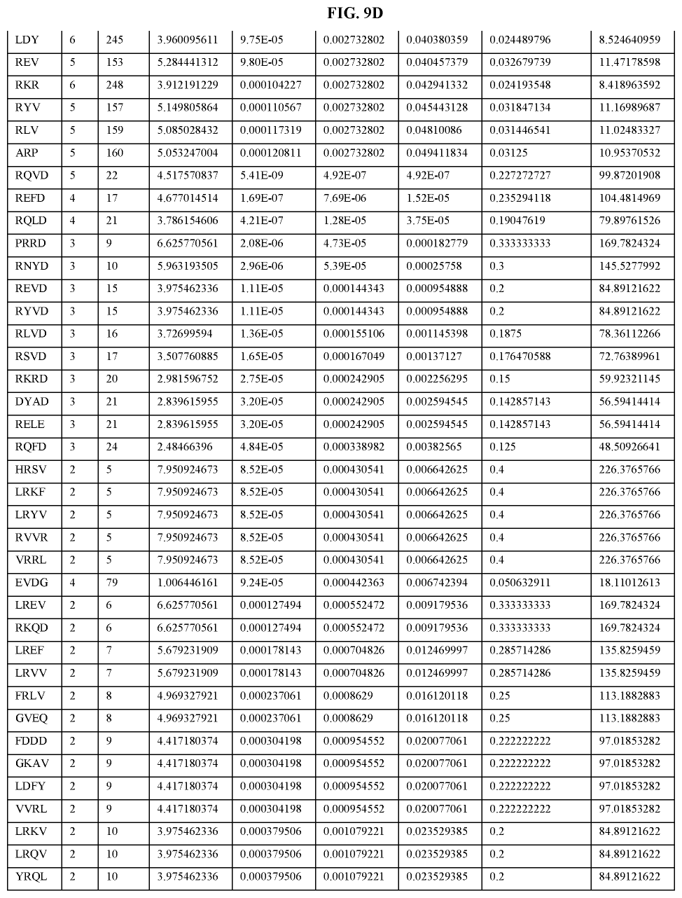

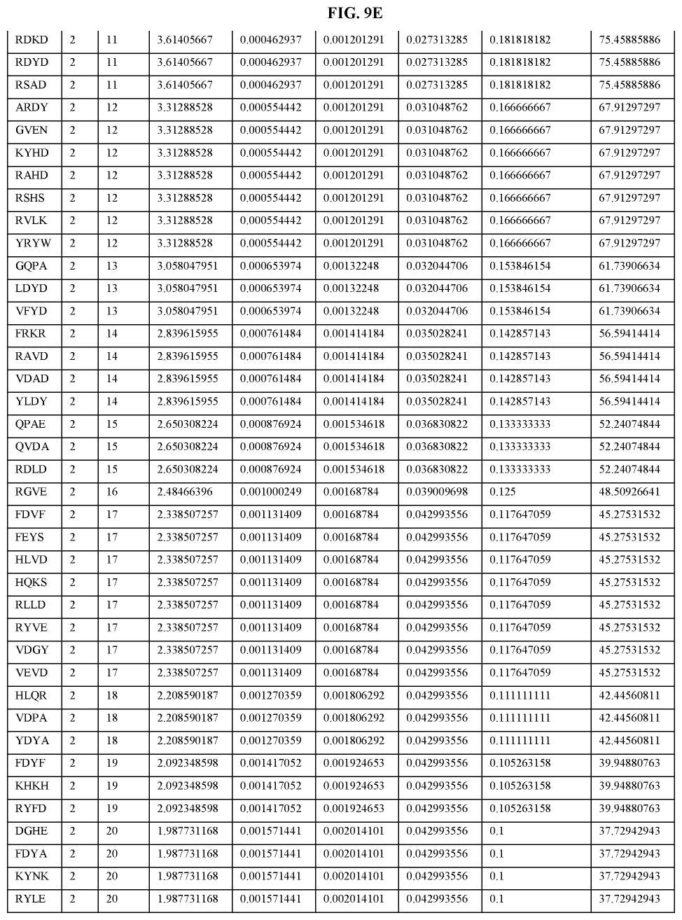

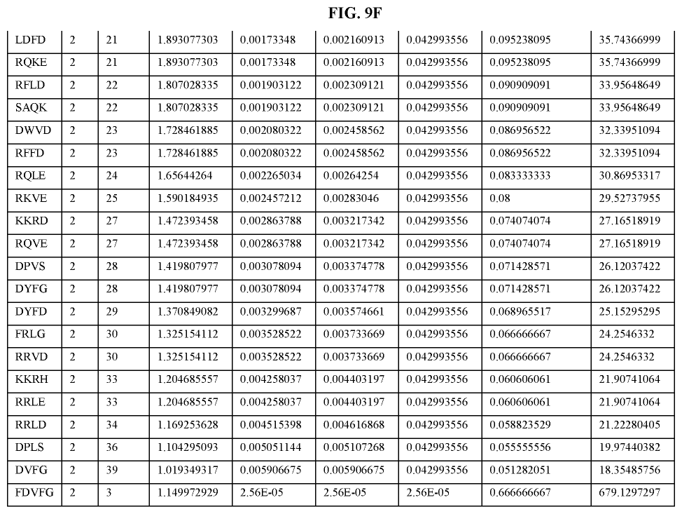

In some embodiments, the infectious disease is Chagas disease, and the discriminating peptides that distinguish Chagas disease in seropositive subjects from healthy reference subjects, which can be subjects that are seronegative for Chagas disease, are enriched in one or more of arginine, aspartic acid, and lysine (FIG. 9A). Enrichment of the one or more amino acids can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total amino acid content of all the peptides in the array library. In some embodiments, discriminating peptides that distinguish Chagas disease from healthy reference subjects are enriched in one or more of motifs provided in FIGS. 9B-F. Enrichment of the one or more amino motifs can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total motif content of all the peptides in the array library.

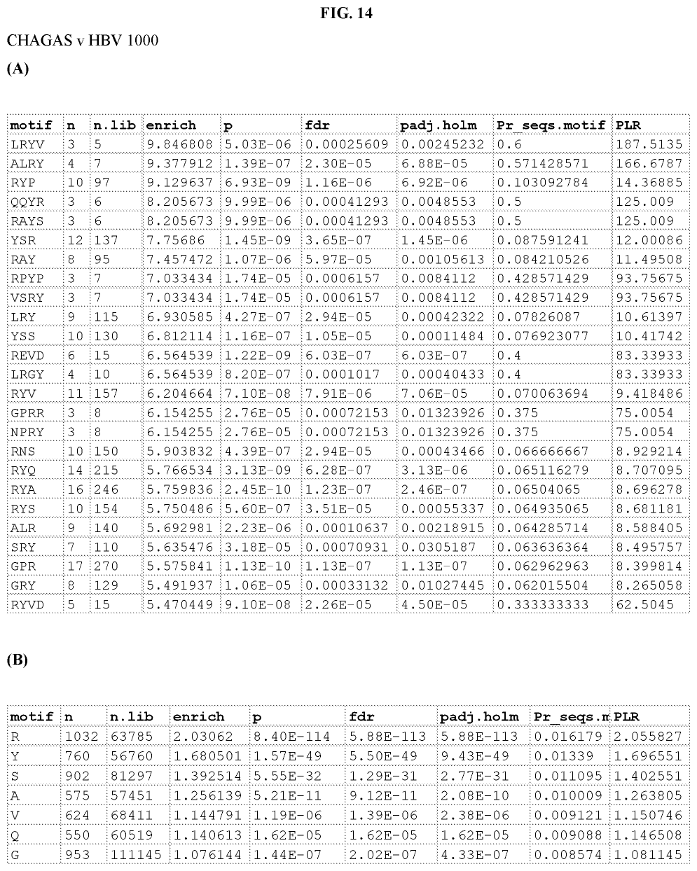

In preferred embodiments, the infectious disease is Chagas disease and the discriminating peptides that distinguish Chagas disease in seropositive subjects from reference subjects that are seropositive for HBV, are enriched in one or more of arginine, tryptophan, serine, alanine, valine, glutamine, and glycine (FIG. 14B). Enrichment of the one or more amino acids can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total amino acid content of all the peptides in the array library. In some embodiments, discriminating peptides that distinguish Chagas disease from HBV reference subjects are enriched in one or more of motifs provided in FIG. 14A. Enrichment of the one or more amino motifs can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total motif content of all the peptides in the array library.

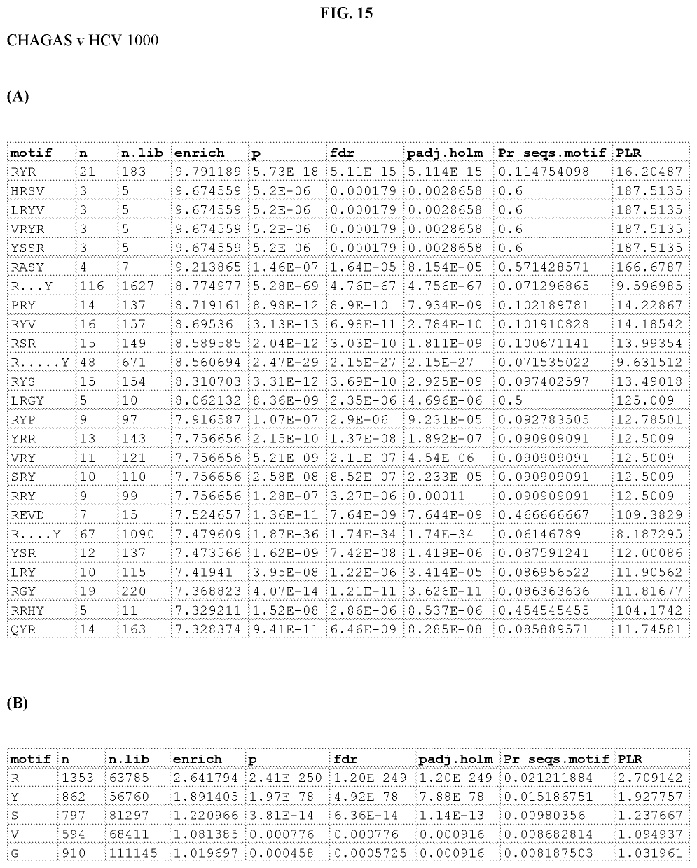

In preferred embodiments, the infectious disease is Chagas disease and the discriminating peptides that distinguish Chagas disease in seropositive subjects from reference subjects that are seropositive for HCV, are enriched in one or more of arginine, tryptophan, serine, valine, and glycine (FIG. 15B). Enrichment of the one or more amino acids can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total amino acid content of all the peptides in the array library. In some embodiments, discriminating peptides that distinguish Chagas disease from reference subjects who are seropositive for HCV are enriched in one or more of motifs provided in FIG. 15 A. Enrichment of the one or more amino motifs can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total motif content of all the peptides in the array library.

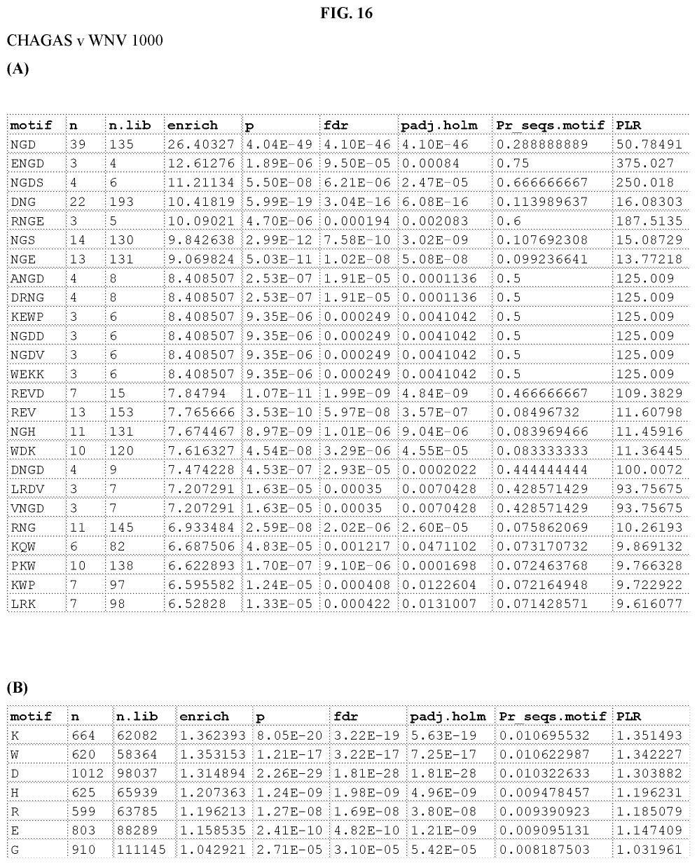

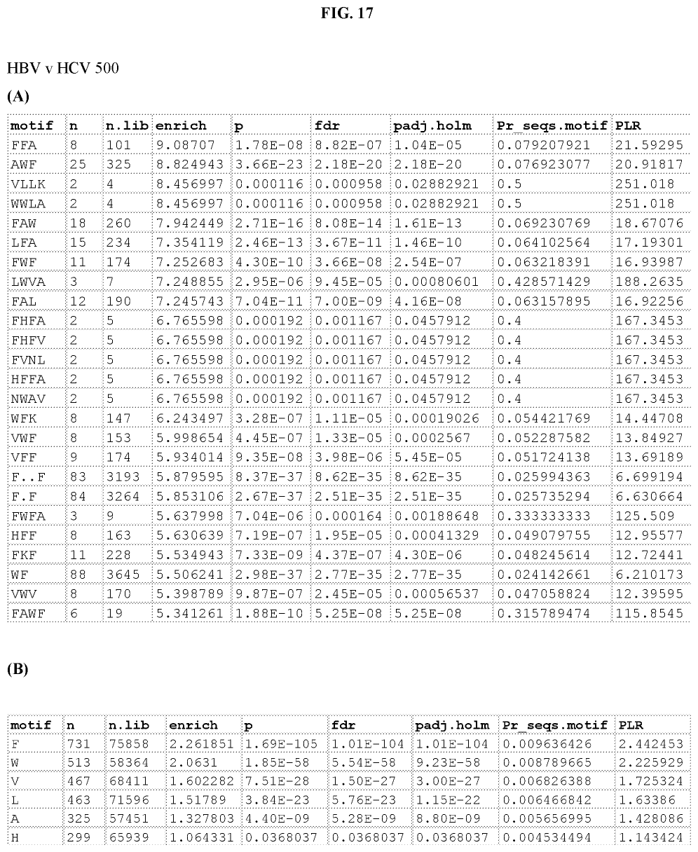

In preferred embodiments, the infectious disease is Chagas disease and the discriminating peptides that distinguish Chagas disease in seropositive subjects from reference subjects that are seropositive for WNV, are enriched in one or more of lysine, tryptophan, aspartic acid, histidine, arginine, glutamic acid, and glycine (FIG. 16B). Enrichment of the one or more amino acids can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total amino acid content of all the peptides in the array library. In some embodiments, discriminating peptides that distinguish Chagas disease from WNV reference subjects are enriched in one or more of motifs provided in FIG. 16A. Enrichment of the one or more amino motifs can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total motif content of all the peptides in the array library.

In preferred embodiments, the infectious disease is HBV disease and the discriminating peptides that distinguish HCV disease in seropositive subjects from reference subjects that are seropositive for WNV, are enriched in one or more of phenylalanine, tryptophan, valine, leucine, alanine, and histidine (FIG. 17B). Enrichment of the one or more amino acids can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total amino acid content of all the peptides in the array library. In some embodiments, discriminating peptides that distinguish HBV disease from HCV reference subjects are enriched in one or more of motifs provided in FIG. 17A. Enrichment of the one or more amino motifs can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total motif content of all the peptides in the array library.

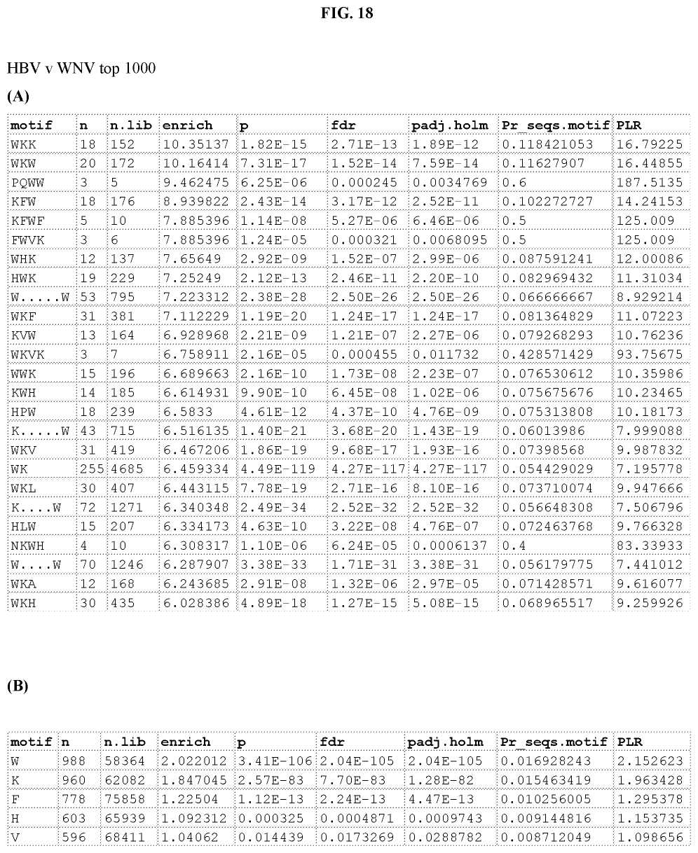

In preferred embodiments, the infectious disease is HBV disease and the discriminating peptides that distinguish WNV disease in seropositive subjects from reference subjects that are seropositive for WNV, are enriched in one or more of tryptophan, lysine, phenylalanine, histidine, and valine (FIG. 18B). Enrichment of the one or more amino acids can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total amino acid content of all the peptides in the array library. In some embodiments, discriminating peptides that distinguish HBV disease from WNV reference subjects are enriched in one or more of motifs provided in FIG. 18A. Enrichment of the one or more amino motifs can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total motif content of all the peptides in the array library.

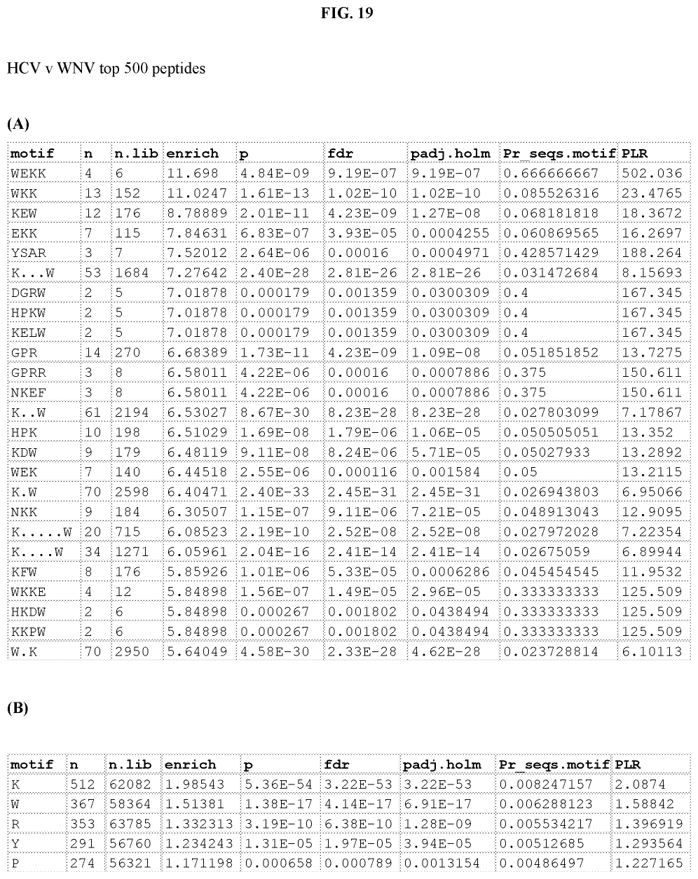

In preferred embodiments, the infectious disease is HCV disease and the discriminating peptides that distinguish HCV disease in seropositive subjects from reference subjects that are seropositive for WNV, are enriched in one or more of lysine, tryptophan, arginine, tyrosine, and proline (FIG. 19B). Enrichment of the one or more amino acids can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total amino acid content of all the peptides in the array library. In some embodiments, discriminating peptides that distinguish HCV disease from WNV reference subjects are enriched in one or more of motifs provided in FIG. 19A. Enrichment of the one or more amino motifs can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total motif content of all the peptides in the array library.

In other embodiments, an individual classifier can be obtained to identify an infection relative to a combined group of two or more different infections, and a combination of discriminating peptides utilized by the classifier is provided. The characteristics of the combination of the discriminating peptides include the prevalence of one or more amino acids, and/or the prevalence of specific sequence motifs present in the identified discriminating peptides. For example, as shown in Example 5, A first binary classifier was created based on discriminating peptides to distinguish subjects that were seropositive for T. cruzii from a group of subjects that were a combination of subjects each being seropositive for HPV, HCV, or WNV. A second binary classifier was created based on discriminating peptides to distinguish subjects that were seropositive for HBV from a group of subjects that were a combination of subjects each being seropositive for Chagas, HCV, or WNV. A third classifier was created based on discriminating peptides to distinguish subjects that were seropositive for HCV from a group of subjects that were a combination of subjects each being seropositive for HPV, Chagas, or WNV. A fourth classifier was created based on discriminating peptides to distinguish subjects that were seropositive for WVN from a group of subjects that were a combination of subjects each being seropositive for HPV, HCV, or Chagas.

Enrichment of amino acid and motif content is relative to the corresponding total amino acid and motif content of all the peptides in the array library. In some embodiments, the discriminating peptides of the immunosignature binding patterns that distinguish a subject with an infectious disease from a group of subjects each subject having one of two or more different infections in diagnosing or detecting an infectious disease in a subject with the methods and arrays disclosed herein are enriched in at least one, at least two, at least three, at least four, at least five, at least six, at least seven, at least eight, at least nine, or at least ten different amino acids. Enrichment of the amino acids can be by greater than 100%, by greater than 125%, by greater than 150%, by greater than 175%, by greater than 200%, by greater than 225%, by greater than 250%, by greater than 275%, by greater than 300%, by greater than 35%, by greater than 400%, by greater than 45%, or by greater than 500% in by greater than one amino acid for the peptides comprising the immunosignature for the infectious disease.

Similarly, in some embodiments, the discriminating peptides of the immunosignature binding patterns for diagnosing or detecting an infectious disease in a subject relative to a group of subjects each having one of two or more different infections with the methods and arrays disclosed herein are enriched in at least one, at least two, at least three, at least four, at least five, at least six, at least seven, at least eight, at least nine, or at least ten different sequence motifs. Enrichment of the sequence motifs can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% in by greater than one motif for the peptides comprising the immunosignature for the infectious disease.

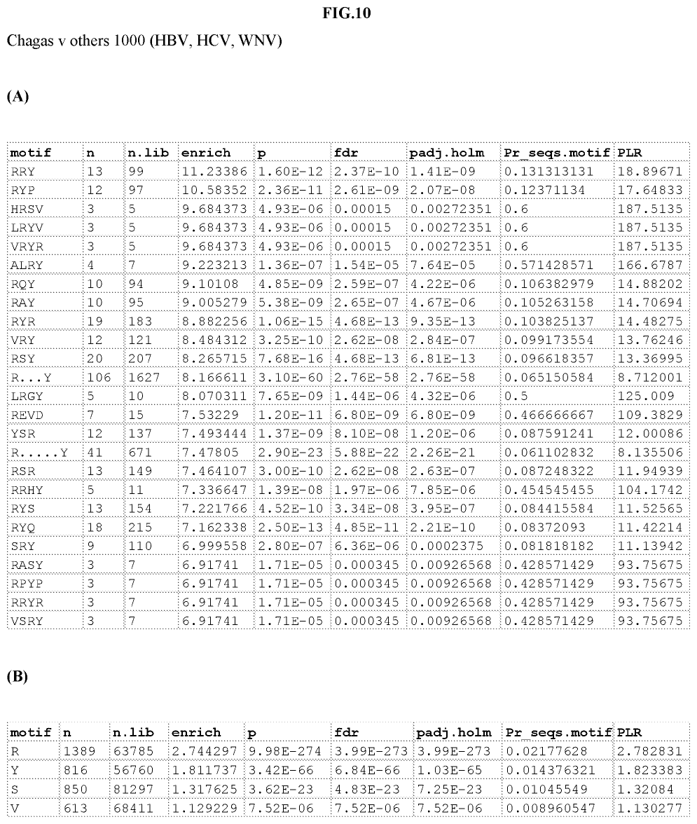

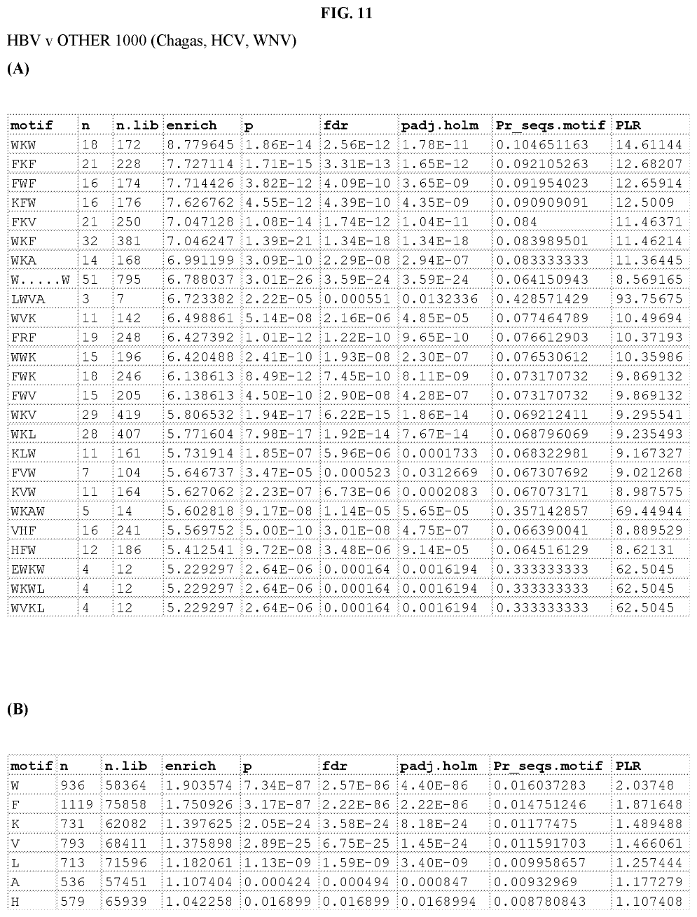

In some embodiments, the infectious disease is Chagas and the discriminating peptides that distinguish Chagas disease in seropositive subjects from a group of reference subjects that are seropositive for one of HBV, HCV and WNV, are enriched in one or more of one or more of arginine, tyrosine, serine and valine (FIG. 10B). Enrichment of the one or more amino acids can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total amino acid content of all the peptides in the array library. In some embodiments, discriminating peptides that distinguish Chagas disease from HBV, HCV and WNV reference subjects are enriched in one or more of motifs provided in FIG. 10A. Enrichment of the one or more amino motifs can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total motif content of all the peptides in the array library.

In some embodiments, the infectious disease is HBV and the discriminating peptides that distinguish HBV disease in seropositive subjects from a group of reference subjects that are seropositive for one of Chagas, HCV and WNV, are enriched in one or more of one or more of tryptophan, phenylalanine, lysine, valine, leucine, arginine, and histidine. (FIG. 11B). Enrichment of the one or more amino acids can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total amino acid content of all the peptides in the array library. In some embodiments, discriminating peptides that distinguish HBV disease from WNV reference subjects are enriched in one or more of motifs provided in FIG. 11A. Enrichment of the one or more amino motifs can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total motif content of all the peptides in the array library.

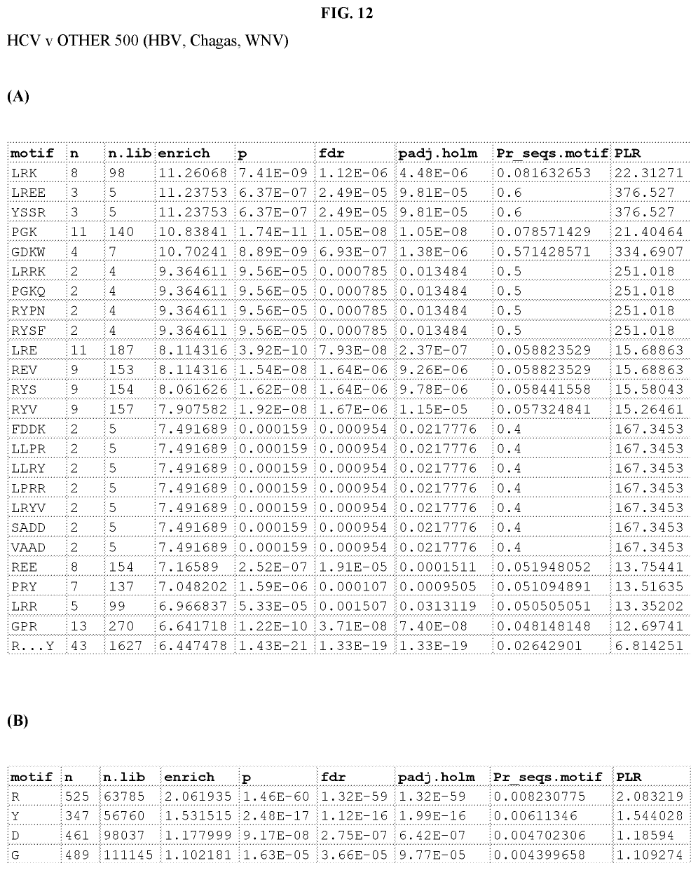

In some embodiments, the infectious disease is HCV and the discriminating peptides that distinguish HCV disease in seropositive subjects from a group of reference subjects that are seropositive for one of Chagas, HBV and WNV, are enriched in one or more of one or more of arginine, tyrosine, aspartic acid, and glycine (FIG. 12B). Enrichment of the one or more amino acids can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total amino acid content of all the peptides in the array library. In some embodiments, discriminating peptides that distinguish HCV disease from reference subjects are enriched in one or more of motifs provided in FIG. 12A. Enrichment of the one or more amino motifs can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total motif content of all the peptides in the array library.

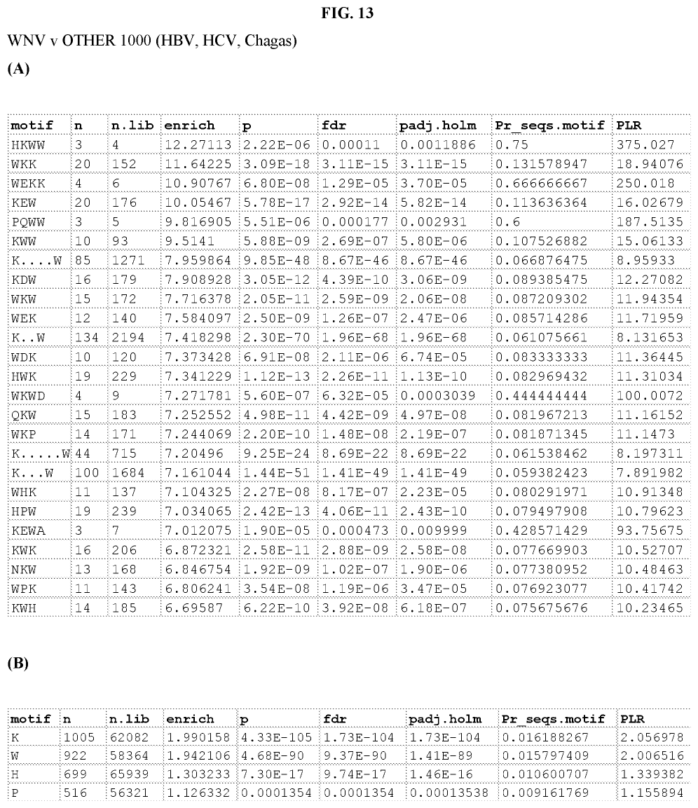

In some embodiments, the infectious disease is WNV and the discriminating peptides that distinguish WNV disease in seropositive subjects from a group of reference subjects that are seropositive for one of Chagas, HBV and HCV, are enriched in one or more of one or more of lysine, tryptophan, histidine, and proline (FIG. 13B). Enrichment of the one or more amino acids can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total amino acid content of all the peptides in the array library. In some embodiments, discriminating peptides that distinguish WNV disease from other reference subjects are enriched in one or more of motifs provided in FIG. 13A. Enrichment of the one or more amino motifs can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% or more, relative to the corresponding total motif content of all the peptides in the array library.

In yet other embodiments, individual classifiers that are independent of each other are obtained based on antibody binding to different sets of discriminating peptides, and combined into a multiclassifer to potentially achieve a best possible classification while increasing the efficiency and accuracy of classification. For example, a first individual classifier based on discriminating peptides that distinguish T. cruzii infection from a reference group of infections HBV, HCV, and WNV, can be combined with a second individual classifier based on discriminating peptides that distinguish HBV from a reference group of infections Chagas, HCV, and WNV, with a third individual classifier based on discriminating peptides that distinguish HCV from a reference group of infections Chagas, HBV and WNV, and with a fourth individual classifier based on discriminating peptides that distinguish WNV from a reference group of infections Chagas, HBV and HCV, to obtain a multiclassifier. Based on the discriminating peptides of each of the individual classifiers, an optimal combination of peptides can emerge to provide a multiclassifier that can simultaneously distinguish two or more different infections from each other. Example 6 demonstrates that the combination of discriminating peptides of the individual classifiers results in a multiclassifier based on a combination of discriminating peptides that can simultaneously distinguish a T. cruzii infection, an HPV infection, an HCV infection, and a WNV infection from each other.

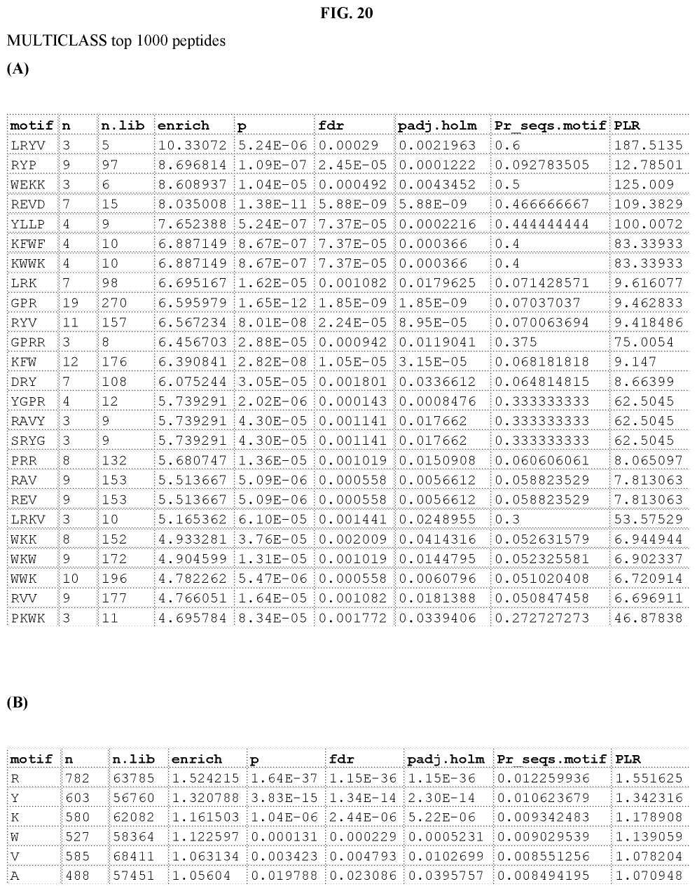

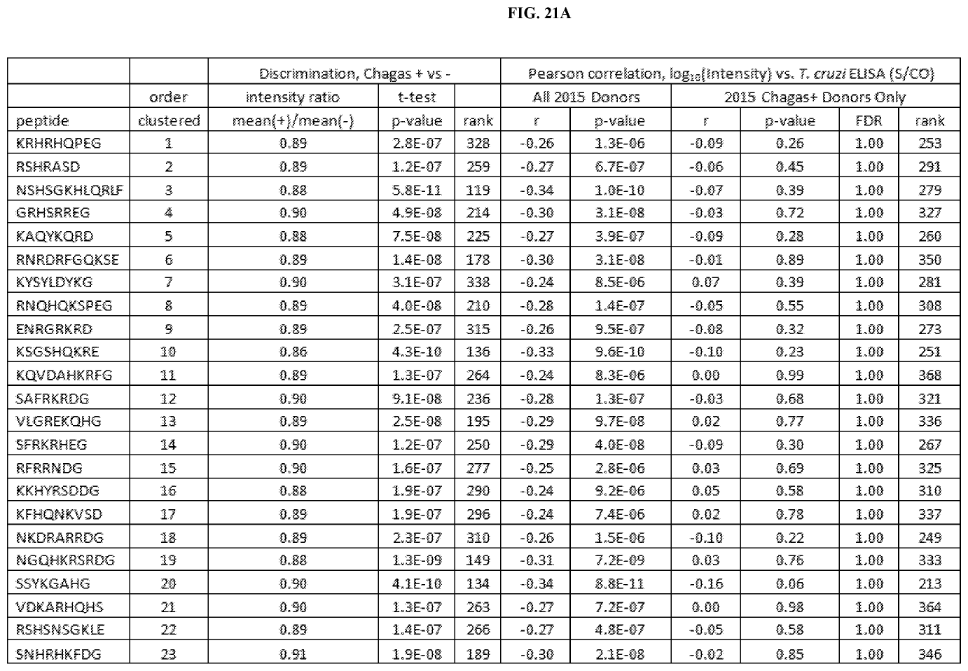

In some embodiments, the discriminating peptides of the immunosignature binding patterns for providing a simultaneous identification of two or more infections in a subject with the methods and arrays disclosed herein are enriched in at least one, at least two, at least three, at least four, at least five, at least six, at least seven, at least eight, at least nine, or at least ten different amino acids. Enrichment of the amino acids can be by greater than 100%, by greater than 125%, by greater than 150%, by greater than 175%, by greater than 200%, by greater than 225%, by greater than 250%, by greater than 275%, by greater than 300%, by greater than 350%, by greater than 400%, by greater than 450%, or by greater than 500% in at least one amino acid for the peptides comprising the immunosignature for the infectious disease. In some embodiments, the simultaneous differentiation is made between Chagas, HBV, HCV, and WNV, wherein discriminating peptides simultaneously distinguish each of these infections from one another. In some embodiments, discriminating peptides that simultaneously distinguish Chagas from each of HBV, HCV, and WNV infections are enriched in one or more of arginine, tyrosine, lysine, tryptophan, valine and alanine (FIG. 20B). In some embodiments, discriminating peptides that simultaneously distinguish HBV from each of Chagas, HCV, and WNV infections are enriched in one or more motifs listed in (FIG. 20A).

### Assay Performance

In some embodiments, the resulting method performance for classifying any infection is characterized by an area under the Radio Operator Characteristic curve (ROC). Specificity, sensitivity, and accuracy metrics of the classification can be determined by the area under the ROC (AUC). In some embodiments, the method determines/classifies the health condition e.g. presence or absence of infection, relative to the serological state of a subject. The performance or accuracy of the method when applied to a plurality of patients whose health condition is already known by alternative methods may be characterized by an area under the receiver operator characteristic (ROC) curve (AUC) being greater than 0.90. In other embodiments, the method performance characterized by an area under the receiver operator characteristic (ROC) curve (AUC) being greater 0.70, greater than 0.80, greater than 0.90, greater than 0.95, method performance characterized by an area under the receiver operator characteristic (ROC) curve (AUC) being greater than 0.97, method performance characterized by an area under the receiver operator characteristic (ROC) curve (AUC) being greater than 0.99. In other embodiments, the method performance is characterized by an area under the receiver operator characteristic (ROC) curve (AUC) ranging from 0.60 to 0.69, 0.70 to 0.79, 0.80 to 0.89, or 0.90 to 1.0. In yet other embodiments, method performance is expressed in terms of sensitivity, specificity, and/or accuracy.

In some embodiments, the method has a sensitivity of at least 60%, for example 65%, 70%, 75%, 80%, 85%, 90%, 910%, 92%, 93%, 94%, 95%, 96%, 97%, 98%, 99% or 100% sensitivity.

In other embodiments, the method has a specificity of at least 60%, for example 65%, 70%, 75%, 80%, 85%, 90%, 91%, 92%, 93%, 94%, 95%, 96%, 97%, 98%, 99% or 100% specificity.

In some embodiments, the method has an accuracy of at least 60%, for example 65%, 70%, 75%, 80%, 85%, 90%, 91%, 92%, 93%, 94%, 95%, 96%, 97%, 98%, 99% or 100%.

Having established an optimal classifier or a multiclassifier model that distinguishes one or more different conditions e.g. the serological state of an individual, the method is applied to determine the condition e.g. the serological state of a subject. A sample is obtained from a subject for whom a diagnosis is desired. The sample is contacted to the array of peptides, and the binding signals resulting from the binding of the antibodies in the subject sample to a plurality of peptides on the array are detected e.g. using a scanner. The images are imported into software to quantitatively compare the binding signal resulting from the binding antibodies in the subject sample to the corresponding binding signal of discriminating peptides previously identified for the optimal classifying model. An overall score that accounts for differences in signals between the discriminating peptides of the model and the binding signals of the corresponding peptides bound by the antibodies of the subject's sample is calculated, and an output indicating for example, the presence or absence of an infection is given. Other outputs can indicate the status of an infection. For example, an output can indicate whether the infection is in an acute state, a chronic state, or an indeterminate state. The status of the infection can be determined for any one of the exemplary infections provided herein i.e. T. cruzi. HBV, HCV, WNV, and any other known infection provided elsewhere herein.

In some embodiments, the method has a reproducibility of classification characterized by an AUC greater than 0.6, greater than 0.65, greater than 0.7, greater than 0.75, greater than 0.80, greater than 0.85, greater than 0.9.0, greater than 0.95, greater than 0.96, greater than 0.97, greater than 0.98, or greater than 0.99. In some embodiments, the reproducibility of classification is characterized by an AUC=1.

### Identifying Candidate Biomarkers

The immunosignature obtained as provided can then be used in multiple applications comprising identifying candidate therapeutic targets, for classifying the infection, monitoring the activity of the infection, and developing treatments for the individual against the identified infectious disorder according to the methods and devices disclosed herein. In another aspect, the differential binding of antibodies in samples from subjects having two or more different health conditions identifies discriminating peptides on the array can be analyzed, for example, by comparing the sequence of one or more discriminating peptides that distinguish between two or more health conditions in the array sequences in a protein database to identify a candidate target protein. In some embodiments, splaying the antibody repertoire out on an array of peptides (immunosignature assay, IST) and comparing samples from diseased subjects e.g. infected subjects, to samples from healthy reference subjects e.g. subjects known not to have an infection, informative discriminating peptides can be identified to reveal the proteins recognized i.e. bound by the antibodies. For example, the peptides can be identified with informatics methods.

In cases where the informatics cannot identify a putative match, such as in the case of discontinuous epitopes, the informative peptide can be used as an affinity reagent to purify reactive antibody. Purified antibody can then be used in standard immunological techniques to identify the target.

Having diagnosed a condition i.e. the infection, the appropriate reference proteome can be queried to relate the sequences of the discriminating peptides bound by the antibodies in a sample. Reference proteomes have been selected among all proteomes (manually and algorithmically, according to a number of criteria) to provide broad coverage of the tree of life. Reference proteomes constitute a representative cross-section of the taxonomic diversity to be found within UniProtKB at http://www.uniprot.org/proteomes/?query=reference:yes Reference proteomes include the proteomes of well-studied model organisms and other proteomes of interest for biomedical and biotechnological research. Species of particular importance may be represented by numerous reference proteomes for specific ecotypes or strains of interest. Examples of proteomes that can be queried include without limitation the human proteome, and proteomes from other mammals, non-mammal animals, viruses, bacteria, fungi, worms, infestations and protozoan parasites. Additionally, other compilations of proteins that can be queried include without limitation lists of disease-relevant proteins, lists of proteins containing known or unknown mutations (including single nucleotide polymorphisms, insertions, substitutions and deletions), lists of proteins consisting of known and unknown splice variants, or lists of peptides or proteins from a combinatorial library (including natural and unnatural amino acids). In some embodiments, the proteomes that can be queried using the identified discriminating peptides include without limitation the proteome of T. cruzi (Sodre C L et al., Arch Microbiol. [2009] February; 191(2):177-84. Epub 2008 Nov. 11. Proteomic map of Trypanosoma cruzi CL Brener: the reference strain of the genome project); the proteomes of HBV, HCV, and WNV which can be found, for example at http://www.uniprot.org/proteomes/.

Software for aligning single and multiple proteins to a proteome or protein list include without limitation BLAST, CS-BLAST, CUDAWS++, DIAMOND, FASTA, GGSEARCH (GG or GL), Genoogle, HMMER, H-suite, IDF, KLAST, MMseqs2, USEARCH, OSWALD, Parasail, PSI-BLAST, PSI_Protein, Sequilab, SAM, SSEARCH, SWAPHI, SWIMM, and SWIPE.

Alternatively, sequence motifs that are enriched in the discriminating peptides relative to the motifs found in the entire peptide library on the array can be aligned to a proteome to identify target proteins that can be validated as possible therapeutic targets for the treatment of the condition. Online databases and search tools for identifying protein domains, families and functional sites are available e.g. Prosite at ExPASy, Motif Scan (MyHits, SIB, Switzerland), Interpro 5, MOTIF (GenomeNet, Japan), and Pfam (EMBL-EBI).

In some embodiments, the alignment method can be any method for mapping amino acids of a query sequence onto a longer protein sequence, including BLAST (Altschul, S. F. & Gish, W. [1996]“Local alignment statistics.” Meth. Enzymol. 266:460-480), the use of compositional substitution and scoring matrices, exact matching with and without gaps, epitope prediction, antigenicity prediction, hydrophobicity prediction, surface accessibility prediction. For each approach, a canonical or modified scoring system can be used, with the modified scoring system optimized to correct for biases in the peptide library composition. In some embodiments, a modified BLAST alignment is used, requiring a seed of 3 amino acids with a gap penalty of 4, with a scoring matrix of BLOSUM62 (Henikoff, J. G. Proc. Natl. Acad. Sci. USA 89, 10915-10919 [1992]) modified to reflect the amino acid composition of the array (States et al., Methods 3:66-70 [1991]). These modifications increase the score of similar substitutions, remove penalties for amino acids absent from the array and score all exact matches equally.

The discriminating peptides that can be used to identify candidate biomarker proteins according to the method provided, are chosen according to their ability to distinguish between two or more different health conditions. As described elsewhere herein, discriminating peptides can be chosen at a predetermined statistical stringency, e.g. by p-value, for the probability of discriminating between two or more conditions; by differences in the relative binding signal intensity changes between two or more conditions; by their intensity rank in a single condition; by their coefficients in a machine learning model trained against two or more conditions e.g. AUC, or by their correlation with one or more study parameters, e.g. R squared, Spearman correlation. In some embodiments, the discriminating peptides selected for identifying one or more candidate biomarkers are chosen as having a p-value of p<1E-03, p<1E-04, or p<1E-05.

Having identified the set of discriminating peptides for an infection as described elsewhere herein, the discriminating peptides are aligned to one or more pathogen proteomes, and peptides having a positive BLAST score are identified. For each of the proteins to which discriminating peptides are aligned, the scores for the BLAST-positive peptides in the alignment are assembled into a matrix e.g. modified BLOSUM62, with each row of the matrix corresponding to an aligned peptide and each column corresponding to one of the consecutive amino acids that comprises the protein.

Each row of the matrix corresponds to an aligned peptide and each column corresponds to an amino acid on the protein, with gaps and deletions allowed within the peptide rows to allow for alignment to the protein.

Using the modified BLAST scoring matrix described above, each position in the matrix receives the score for paired amino acids of the peptide and protein in that column. Then, for each amino acid in the protein, the corresponding column is summed to create an amino acid “overlap score” that represents coverage of that amino acid at a position in the protein by the discriminating peptides.

The amino acid overlap score is subsequently corrected for the composition i.e. the amino acid content of the array library. For example, a correction is made to account for library array peptides that exclude one or more of the 20 natural amino acids. To correct this score for library composition, an amino acid overlap score is calculated by the same method for a list of all array peptides. This allows for the calculation of a peptide overlap difference score based on the discriminating peptides, s_{d}, at each amino acid position according to the following equation:

s_{d}=a−(b/d)*c

where “a” is the overlap score from the discriminating peptides, “b” is the number of ImmunoSignature discriminating peptides, “c” is the overlap score for the full array of peptide and “d” is the number of library peptides on the entire array.

Next, the amino acid overlap score obtained from the alignment of the discriminating peptides is converted to a protein score, S_{d}. To convert the scores at the amino acid level, S_{d}, to a full-protein statistic, S_{d}, the sum of scores for every possible tiling n-mer epitope within a protein is calculated, and the final score is the maximum score obtained along this rolling window of n-mers for each protein, where n can be 20 (etc). In some embodiments, the scores can be obtained for tiling 10-mer epitopes, 15-mer-epitopes, 20-mer epitopes, 25-mer epitopes, 30 mer-epitopes, 35-mer-epitopes, 40-mer-epitopes, 45-mer epitopes, or 50-mer epitopes. Protein score S_{d} is the maximum score obtained along the rolling window. In some embodiments, the n-mer correlates to the entire length of the protein i.e. the discriminating peptides are aligned to the entire sequence of the protein. Alternatively, the scores can be obtained by aligning the peptide sequences to the entire protein sequences.

Ranking of the identified candidate biomarkers is made subsequently relative to the ranking of randomly chosen non-discriminating peptides. Accordingly, an overlap score for non-discriminating peptides (non-discriminating random ‘sᵣ’ score) i.e. randomly chosen peptides that align to each of one or more proteins of a same proteome or protein list is obtained as described for the discriminating peptides. The amino acid overlap score is calculated for the random peptides, and is subsequently corrected for amino acid content of the peptide library to provide a non-discriminating or random sᵣ score. The non-discriminating sᵣ score is then converted to a non-discriminating protein ‘Sᵣ’ score for each of a plurality of randomly chosen non-discriminating peptides. For example, non-discriminating random protein ‘Sᵣ’ scores can be obtained for at least 25, at least 50, at least 100, at least 150, at least 200, or more randomly-chosen non-discriminating peptides. In some embodiments, the final protein score, Sᵣ score-for the randomly chosen non-discriminating peptides can be calculated using the equivalent number of discriminating peptides used to obtain protein score S_{d}. In other embodiments, at least 20%, at least 30%, at least 40%, at least 50%, at least 60%, at least 70%, at least 80%, at least 85%, at least 90%, at least 95%, at least 98%, at least 99% of the number of discriminating peptides used to determine S_{d} are used to determine the non-discriminating protein ‘Sᵣ’ score.

In some embodiments, the candidate protein biomarkers are ranked by their S_{d} score relative to the S, score of the proteins identified by alignment of non-discriminating peptides. In some embodiments, ranking can be determined according to a p-value. Top candidate biomarkers can be chosen as having a p-value less than 10⁻³, less than 10⁻⁴, less than 10⁻⁵, less than 10⁻⁶, less than 10⁻⁷, less than 10⁻⁸, less than 10⁻⁹, less than 10⁻¹⁰, less than 10⁻¹², less than 10⁻¹⁵, less than 10⁻¹⁸, less than 10⁻²⁰, or less. In some embodiments, at least 5, at least 10, at least 15, at least 20, at least 30, at least 40, at least 50, at least 60, at least 70, at least 80, at least 90, at least 100, at least 120, at least 150, at least 180, at least 200, at least 250, at least 300, at least 350, at least 400, at least 450, at least 500, or more candidate biomarkers are identified according to the method.

In other embodiments, candidate biomarkers are chosen according to the S_{d} score obtained by tiling a plurality of discriminating peptides to n-mer epitopes as described in the preceding paragraphs, and selecting the number of candidate biomarkers as a percent of proteins having the greatest S_{d} score for the pathogen's proteome. In some embodiments, candidate biomarkers are proteins having the highest ranking S_{d} scores and comprising at least 0.01% of the total number of proteins of the pathogens' proteome. In other embodiments, candidate biomarkers are proteins having the highest ranking S_{d} scores and comprising at least 0.02%, at least 0.03%, at least 0.04%, at least 0.05%, at least 0.1%, at least 0.15%, at least 0.2%, at least 0.25%, at least 0.3%, at least 0.35%, at least 0.4%, at least 0.45%, at least 0.5%, at least 0.55%, at least 0.6%, at least 0.65%, at least 0.7%, at least 0.75%, at least 0.8%, at least 0.85%, at least 0.9%, at least 1%, at least 2%, at least 3%, at least 4%, at least 5%, at least 10%, at least 20%, or more of the total number of proteins of the pathogens' proteome.

In some embodiments, a method is provided for identifying at least one candidate protein biomarker for an infection in a subject, the method comprising: (a) providing a peptide array and incubating a biological sample from said subject to the peptide array; (b) identifying a set of discriminating peptides bound to an antibody in the biological sample from said subject, the set of peptides capable of differentiating the infection from at least one different condition; (c) querying a proteome database with a plurality of said discriminating peptides in said set; (d) aligning said plurality of peptides in said set to one or more proteins of the proteome of the infection-causing pathogen; and (e) obtaining a relevance score for each of the proteins and ranking for each of the identified proteins from the proteome database; wherein each of the identified proteins is a candidate biomarker for the disease in the subject. In some embodiments, the at least one different condition can comprise one or more different infections, and/or a healthy condition. In some embodiments, the method further comprises obtaining an overlap score, wherein said score corrects for the peptide composition of the peptide library. The discriminating peptides can be identified by statistical means e.g. t-test, as having p-values of less than 10⁻³, less than 10⁻⁴, less than 10⁻⁵, less than 10⁻⁶, less than 10⁻⁷, less than 10⁻⁸, less than 10⁻⁹, less than 10⁻¹⁰, less than 10⁻¹¹, less than 10⁻¹², less than 10⁻¹³, less than 10⁻¹⁴, or less than 10⁻¹⁵. In some embodiments, the resulting candidate biomarkers can be ranked according to a p-value of less than less than 10⁻³, less than less than 10⁻⁴, less than less than 10⁻⁵, or less than less than 10⁻⁶ when compared to proteins identified according to the method but using non-discriminating peptides.

Candidate Biomarkers of Infectious Disease e.g. Chagas Disease

Example 4 illustrates a method for identifying candidate proteins biomarkers using discriminating peptides that distinguish the serological state of samples form healthy subjects from samples from subjects infected with T. cruzi (Chagas disease). Healthy subjects can be subjects that were previously infected with T. cruzi and have seroreconverted to being seronegative, and/or subjects that have never been infected with T. cruzi. A list of candidate protein biomarkers is provided in Table 2. Similarly, candidate protein biomarkers can be identified using discriminating peptides that distinguish the serological state of samples from subjects having other infectious diseases from samples from healthy subjects, from samples from subjects having other infectious diseases, and from samples from subjects having mimic diseases, which may or may not be infectious.

In some embodiments, a method for identifying a candidate protein biomarker for an infectious disease comprises: (a) providing a peptide array and incubating a biological sample from said subject to the peptide array; (b) identifying a set of discriminating peptides bound to antibodies in the biological sample from the subject, the set of discriminating peptides displaying signals capable of differentiating the samples that are seropositive for the infectious disease from samples that are seronegative for the same infectious disease; (c) querying a proteome database with each of the peptides in the set of discriminating peptides; (d) aligning each of the peptides in the set of peptides to one or more proteins in the proteome database to identify one or more proteins of the pathogen causing the infection; and (e) obtaining a relevance score and ranking for each of the identified proteins from the proteome database; wherein each of the identified proteins is a candidate biomarker for the infectious disease in the subject. In some embodiments, the discriminating peptides used in the method are identified as having p-values of less than 10⁻⁵, less than 10⁻⁶, less than 10⁻⁷, less than 10⁻⁸, less than 10⁻⁹, less than 10⁻¹⁰, less than 10⁻¹¹, less than 10⁻¹², less than 10⁻¹³, less than 10⁻¹⁴, or less than 10⁻¹⁵. In other embodiments, the discriminating peptides used in the method are all of the discriminating peptides, i.e. peptides that have not been ranked according to a statistical method.

In some embodiments, the method further comprises identifying a set of discriminating peptides that differentiate the infectious disease from a healthy condition e.g. a seronegative condition. In some embodiments, the discriminating peptides distinguish from subjects having Chagas from subjects having a different infection. Alternatively, the discriminating peptides distinguish subjects having Chagas from a mixture of subjects each having a different infection. In some embodiments, subjects with any one infection e.g. Chagas, HBV, HCV, WNV, can be distinguished from subjects not having an infection. In some instances the subjects not having the infection are seronegative subjects that have reversed from having an infection. Thus, the candidate biomarkers can serve to diagnose a disease, and to identifying a stage of disease progression. The biomarkers can also be used in the monitoring of infectious diseases. Examples of candidate biomarkers identified in subjects having Chagas relative to healthy subjects are listed in Table 2. In some embodiments, the candidate biomarker proteins identified according to the method are ranked according to a p-value of less than less than 10⁻³, less than less than 10⁻⁴, less than less than 10⁻⁵, or less than less than 10⁻⁶. Ranking of the resulting candidate can be determined relative to proteins that have been identified from array peptides that are non-discriminating for a condition.

Alternatively, discriminating peptides identified according to the methods provided, can identify candidate target proteins using sequence motifs that are enriched in the most discriminating peptides that distinguish two different conditions. In one embodiment, the method for identifying a candidate target for the treatment of an infectious disease in a human subject comprises (a) obtaining a set of discriminating peptides that differentiate the infectious disease from one or more different infectious diseases; (b) identifying a set of motifs for said discriminating peptides; (c) aligning the set of motifs to a human proteome; (d) identifying regions of homology between each motif in the set to a region of an immunogenic protein; and (e) identifying the protein as a candidate target for treating said infectious disease. The method can further comprise identifying a set of discriminating peptides that differentiate the infectious disease from a healthy condition. Motifs that are enriched in the most discriminating peptides that can be used to identify candidate target proteins for development and use in treating various infectious diseases, some at different stages of progression are provided in FIGS. 9-20.

In some embodiments, the step of identifying the discriminating peptides can comprise (i) detecting the binding of antibodies present in samples form a plurality of subjects having said infectious disease to an array of different peptides to obtain a first combination of binding signals; (ii) detecting the binding of antibodies to a same array of peptides, said antibodies being present in samples from two or more reference groups of subjects, each group having a different health condition; (iii) comparing said first to said second combination of binding signals; and (iv) identifying peptides on said array that are differentially bound by antibodies in samples from subjects having said disease and the antibodies in said samples from two or more reference groups of subjects, thereby identifying said discriminating peptides. In some embodiments, the infectious disease is Chagas disease. In some embodiments, Chagas is distinguished from a healthy condition. In some embodiments, Chagas is distinguished from one or more different infections. As described above, infections such as HBV, HCV, WNV and Chagas can be distinguished from one another.

### Applications for Candidate Biomarkers

In other embodiments, the methods, apparatus and systems provided identify discriminating peptides that correlate with disease activity, and/or correlate with changes in disease activity over time. For example, discriminating peptides can determine disease activity and correlate it with the activity defined by known markers of an existing scoring system. Example 3 describes that several discriminating peptides correlate to the S/CO activity score for Chagas. These discriminating peptides have been used to identify proteins according to the method provided. Therefore, some of these proteins may be novel candidate biomarkers that can be used in tests and monitoring of Chagas disease activity.

The discriminating peptides can also serve as a basis for the design of drugs that inhibit or activate the target protein-protein interactions. In another aspect, therapeutic and diagnostic uses for the novel discriminating peptides identified by the methods of the invention are provided. Aspects and embodiments thus include formulations, medicaments and pharmaceutical compositions comprising the peptides and derivatives thereof according to the invention. In some embodiments, a novel discriminating peptide or its derivative is provided for use in medicine. More specifically, for use in antagonising or agonising the function of a target ligand, such as a cell-surface receptor. The discriminating peptides of the invention may be used in the treatment of various diseases and conditions of the human or animal body, such as cancer, and degenerative diseases. Treatment may also include preventative as well as therapeutic treatments and alleviation of a disease or condition.

Accordingly, the methods, systems and array devices disclosed herein are capable of identifying discriminating peptides, which serve to identify candidate biomarkers, identify vaccine targets, which in turn are useful for medical interventions for treating a disease and/or condition at an early stage of the disease and/or condition. For example, the methods, systems and array devices disclosed herein are capable of detecting, diagnosing and monitoring a disease and/or condition days or weeks before traditional biomarker-based assays. Moreover, only one array, i.e., one immunosignature assay, is needed to detect, diagnose and monitor a side spectra of diseases and conditions caused by infectious agents, including inflammatory conditions, autoimmune diseases, cancer and pathogenic infections. The candidate biomarkers can be identified for validation and subsequent development of therapeutics.

### Infectious Diseases

The assays, methods and devices provided can be utilized to identify a plurality of different infections. In some embodiments, the assays, methods and devices provided can be utilized to identify discriminating peptides that distinguish any one infection from any other one or more infections. In other embodiments, the discriminating peptides that identify the different infections can be utilized to identify candidate biomarkers for the different infections. The methods, apparatus, and devices described herein are suitable for identifying infections caused by a wide variety of pathogens including bacteria, viruses, fungi, protozoans, worms, and infestations, In some embodiments, the assays, methods and devices provided can be utilized to identify candidate biomarkers for medical intervention of the different infections, including diagnosing an infection, providing a differential diagnosis of an infection relative to other infections and diseases mimicking those caused by the infections, determining the progression of the infection and disease caused thereby, scoring the activity of the infection and disease, serving as candidate target for evaluation as therapeutics for the treatment of the infection and disease, and stratifying patients in clinical trials based on predicted responses to therapy.

The candidate biomarkers can be utilized in the medical intervention of any infectious disease.

In some embodiments, the infection is caused by a pathogenic viral infection for which candidate biomarkers can be identified according to the methods provided. Non-limiting examples of pathogenic viral infections for which candidate biomarkers can be identified according to the methods provided include infections caused viruses that can be found in the following families of viruses and are illustrated with exemplary species: a) Adenoviridae family, such as Adenovirus species; b) Herpesviridae family, such as Herpes simplex type 1, Herpes simplex type 2, Varicella-zoster virus, Epstein-barr virus, Human cytomegalovirus, Human herpesvirus type 8 species; c) Papillomaviridae family, such as Human papillomavirus species; d) Polyomaviridae family, such as BK virus, JC virus species; e) Poxviridae family, such as Smallpox species; f) Hepadnaviridae family, such as Hepatitis B virus species; g) Parvoviridae family, such as Human bocavirus, Parvovirus B19 species; h) Astroviridae family, such as Human astrovirus species; i) Caliciviridae family, such as Norwalk virus species; j) Flaviviridae family, such as Hepatitis C virus, yellow fever virus, dengue virus, West Nile virus species; k) Togaviridae family, such as Rubella virus species; 1) Hepeviridae family, such as Hepatitis E virus species; m) Retroviridae family, such as Human immunodeficiency virus (HIV) species; n) Orthomyxoviridaw family, such as Influenza virus species; o) Arenaviridae family, such as Guanarito virus, Junin virus, Lassa virus, Machupo virus, and/or Sabia virus species; p) Bunyaviridae family, such as Crimean-Congo hemorrhagic fever virus species; q) Filoviridae family, such as Ebola virus and/or Marburg virus species; Paramyxoviridae family, such as Measles virus, Mumps virus, Parainfluenza virus, Respiratory syncytial virus, Human metapneumovirus, Hendra virus and/or Nipah virus species; r) Rhabdoviridae genus, such as Rabies virus species; s) Reoviridae family, such as Rotavirus, Orbivirus, Coltivirus and/or Banna virus species; t) Flaviviridae family, such as Zika Virus. In some embodiments, a virus is unassigned to a viral family, such as Hepatitis D.

In some embodiments, the infections are bacterial infections caused by pathogens including Streptococcus (pyogenes, viridans). Staphylococcus (aureus, epidermidis, saprophyticus). Pseudomonas aeruginosa, Burkholderia cenocepacia, Mycobacterium (M. leprae. M. tuberculosis, avium). Actinomyces israelii. Bacillus anthracis. Bacteroides fragilis. Bordetella pertussis. Borrelia (B. burgdorferi. B. garinii. B. afzelii). Campylobacter jejuni. Chlamydia (C. pneumoniae. C. trachomatis). Chlamydophila psittaci. Clostridium (C. botulinum C. difficile. C. perfringens. C. tetani). Enterococcus (E. faecalis. E. faecium). Escheridia (E. coli. Enterotoxigenic E. coli. Enteropathogenic E. coli. Enteroinvasive E. coli. Enterohemorrhagic (EHEC), including E. coli O157:H7). Francisella tularensis. Haemophilus influenzae. Helicobacter pylori. Klebsiella pneumoniae. Legionella pneumophila, Leptospira species. Mycoplasma pneumoniae. Nocardia asteroides. Shigella (S. sonnel. S. dysenteriae) Treponema pallidum, and Vibrio cholerae. Obligate intracellular parasites (e.g. Chlamydophila, Ehrlichia (E. canis. E. chaffeensis), Rickettsia. Salmonella (S. typhi, other Salmonella species e.g. S. typhimurium), Neisseria (N. gonorrhoeae, N. meningitides), Brucella (B. abortus, B. canis, B. melitensis, B. suis), Mycobacterium, Nocardia, Listeria Listeria monocytogenes, Francisella, Legionella, and Yersiniapestis. Infections caused by bacterial pathogens further include sexually transmittable disease including Chancroid caused by Haemophilus ducreyi, Chlamydia caused by Chlamydia trachomatis), Gonorrhea (Neisseria gonorrhoeae), Granuloma inguinale or (Klebsiella granulomatis), Mycoplasma genitalium, Mycoplasma hominis, Syphilis (Treponema pallidum), and Ureaplasma infection.

In some embodiments, the subject suffers from a protozoan infection, which are parasitic diseases caused by organisms formerly classified in the Kingdom Protozoa. They include organisms classified in Amoebozoa, Excavata, and Chromalveolata. Examples include Entamoeba histolytica, Acanthamoeba: Balamuthia mandrillaris:and Endolimax: Plasmodium (some of which cause malaria), and Giardia lamblia.^{[2]} Trypanosoma brucei, transmitted by the tsetse fly and the cause of African sleeping sickness, is another example. Other non-limiting examples of protozoa can be found in the following families and are illustrated with exemplary species: a) Trypanosoma cruzi species; Trypanosoma brucei species; Toxoplasma gondii species; Plasmodium falciparum species; Entamoeba histolytica species, and Giardia lamblia species. The capability of the method provided to identify candidate biomarkers for an infectious disease is demonstrated in the Examples, which show that discriminating peptides can identify candidate biomarkers in samples from subjects infected with the protozoan Trypanosoma cruzi, which causes Chagas disease, also known as American trypanosomiasis.

In other embodiments, the infection is a fungal infection i.e. mycosis, including superficial mycoses, cutaneous mycoses, subcutaneous mycoses, systemic mycoses due to primary pathogens, and systemic mycoses due to pathogenic fungi including the candida sp., Aspergillus sp., Cryptoccocus sp., Histoplasma sp., Pneumocystis sp., Stachybitrys sp., and Endothermy sp.

In other embodiments, the infection is a transmissible spongiform encephalopathy (TSE), which belongs to a group of progressive conditions that affect the brain (encephalopathies) and nervous system of many animals, including humans, and are caused by infection by prions, which are transmittable pathogenic agents. According to the most widespread hypothesis, they are transmitted by prions, though some other data suggest an involvement of a Spiroplasma infection. Prion diseases of humans include classic Creutzfeldt-Jakob disease, new variant Creutzfeldt-Jakob disease (nvCJD, a human disorder related to bovine spongiform encephalopathy), Gerstmann-Sträussler-Scheinker syndrome, fatal familial insomnia, kuru, and the recently discovered variably protease-sensitive prionopathy.

In some embodiments, the infection is a parasitic helminthiasis, also known as worm infection, which is any macroparasitic disease of humans and other animals in which a part of the body is infected with parasitic worms, known as helminths. There are numerous species of these parasites, which are broadly classified into tapeworms, flukes, and roundworms. They often live in the gastrointestinal tract of their hosts, but they may also burrow into other organs, where they induce physiological damage. Of all the known helminth species, the most important helminths with respect to understanding their transmission pathways, their control, inactivation and enumeration in samples of human excreta from dried feces, faecal sludge, wastewater, and sewage sludge are: soil-transmitted helminths, including Ascaris lumbncoides (the most common worldwide), Trichuris trichiura, Necator americanus, Strongyloides stercoralis and Ancylostoma duodenale: Hymenolepis nana; Taenia saginata: Enterobius; Fasciola hepatica; Schistosoma mansoni; Toxocara canis; and Toxocara cati. Helminthiases are classified as follows (the disease names end with “-sis” and the causative worms are in brackets); Roundworm infection (nematodiasis): Filariasis (Wuchereria bancrofti, Brugia malayi infection); Onchocerciasis (Onchocerca volvulus infection); Soil-transmitted helminthiasis—this includes ascariasis (Ascaris lumbricoides infection, trichuriasis (Trichuris infection), and hookworm infection (includes Necatoriasis and Ancylostoma duodenale infection); Trichostrongyliasis (Trichostrongylus spp. infection); Dracunculiasis (guinea worm infection); Tapeworm infection (cestodiasis); Echinococcosis (Echinococcus infection); Hymenolepiasis (Hymenolepis infection); Taeniasis/cysticercosis (Taenia infection); Coenurosis (T. multiceps, T. serialis, T. glomerata, and T. brauni infection); Trematode infection (trematodiasis); Amphistomiasis (amphistomes infection); Clonorchiasis (Clonorchis sinensis infection); Fascioliasis (Fasciola infection); Fasciolopsiasis (Fasciolopsis buski infection); Opisthorchiasis (Opisthorchis infection); Paragonimiasis (Paragonimus infection); Schistosomiasis/bilharziasis (Schistosoma infection); and Acanthocephala infection: Moniliformis infection.

In other embodiments, the infection is a tickborne infection including Anaplasmosis, babesiosis, ehrlichiosis, lyme disease (Borrelia burgorferi infecton), Powassan virus infection, spotted fever rickiettiosis, including Rocky Mountain spotted fever (RMSF), and typhus fever.

The timeline for infectious organisms and corresponding symptomatic changes in individuals may vary for each disease. In Chagas disease, for example, an infected individual initially experiences an acute phase of 4-8 weeks that manifests as periorbital swelling or ulcerative lesions at the entry site and is associated with high-levels of parasite circulating through the bloodstream. This transitions into the asymptomatic, indeterminant phase that is typically a life-long infection and that is characterized by loss of blood-parasitemia and sequestration of the protozoa into muscle and fat cells of host organs [Perez C J et al., Lymbery A J, Thompson R C (2014) Trends Parasitol 30: 176-182.]. Ten to thirty years later, a third or more of the individuals in the indeterminate phase will progress to a chronic, symptomatic phase, and will suffer severe manifestations of cardiac, gastric, or other organ-related disease that lead to irreversible muscular lesions and often death within two years of entering the chronic phase [Viotti R et al., (2006) Ann Intern Med 144: 724-734; Granjon E et al., (2016) PLoS Negl Trop Dis 10: e0004596; Oliveira G B F et al., (2015) Global Heart 10: 189-192]. Additionally, reactivation of Chagas disease has been documented in immunocompromised patients including patients co-infected with HIV or patients under treatment for cancer or autoimmune disorders [Rassi Jr A et al., (2010); Pinazo M J et al., (2013) PLoS Negl Trop Dis 7: e1965].

The WHO has recently estimated that approximately 200,000 people will die from Chagasic cardiomyopathy in the next five years. That corresponds to the same number of women forecast to die in the US from breast cancer in the same timeframe [Pecoul B et al., (2016) PLoS Negl Trop Dis 10: e0004343].

There is no vaccine against Chagas and the only mode of prevention is to control spread of the insect-vector. For the past 40 years only two drugs, benznidazole and nifurtimox, have been available for treatment [Rassi Jr A et al., (2010), Clayton J (2010) Nature 465: S4-S5]. They have shown variable but significant effectiveness against acute phase infections but have proven little therapeutic value to those suffering chronic manifestations or for preventing transition from subclinical to symptomatic disease [Issa V S and Bocchi E A (2010) The Lancet 376: 768; Morillo C A et al., (2015) New England Journal of Medicine 373: 1295-1306.]. The unpredictability of the drugs' efficacy, poor availability, and known side-effects have rendered their prescription to less than 1% of diagnosed Chagas patients [Clayton J (2010); Viotti R et al., (2009) Expert Rev Anti Infect Ther 7: 157-163]. Some that receive treatment experience adverse complications that require stoppage [Viotti R et al., (2006)]. There is currently no tool to identify which patients would benefit versus be harmed by treatment.

Recently, there has been some increased interest in discovering new drugs against T. cruzi infections that are safer and more efficacious [De Rycker M et al., (2016) PLoS Negl Trop Dis 10: e0004584.]. However, development of new drugs has been hampered by the lack of reliable, and practical methods to assess drug efficacy at the subclinical and chronic phases. Many difficulties exist in measuring infection status and determining therapeutic impact [Gomes Y M et al., (2009) Mem Inst Oswaldo Cruz 104 Suppl 1: 115-121]. For example, parasitemia is subpatent and low levels of tissue-parasites are anatomically scattered, the existence of antigen similarity to other endemic diseases such as leischmaniosis and malaria, the absence of reliable markers of incipient or active disease, and the lag in the development of symptoms by decades post initial infection [Keating S M et al., et al. (2015) Int J Cardiol 199: 451-459.] In sum, a method is needed to stratify Chagas seropositive individuals into clinically distinct groups. For example, it would be important to distinguish those individuals who remain infected following the acute phase from those that have resolved it. Therefore, it would be desirable to predict which of the infected individuals in the indeterminant phase individuals will progress from being clinically silent to having life-threatening complications.

Direct detection of the T. cruzii parasite can be done by blood microscopy, hemoculture, xenodiagnosis, or PCR of nucleic acids extracted from peripheral blood cells. However, these assays are not sensitive, and are considered uninformative in the chronic disease phase. In clinics and blood banks, diagnosis is dependent on indirect detection by serology. ELISA tests are available for the detection of T. cruzi antibodies against crude parasite lysate (Ortho T. cruzi ELISA), semi-purified in vitro-cultured epimastigote fractions, or a mix of four recombinant proteins (Abbott PRISM and ESA Dot Blot). The FDA has approved the Ortho and Abbott tests, which report a signal to cut off value (S/CO) for Chagas Disease that quantifies levels of antigen binding in blood plasma and reflect antibody titers. Unfortunately, inconclusive and discordant results both between and within these test platforms are persistent problems, as are cross-reactivity and the common occurrence of false positives. Consequently, confirmatory serologic tests are used in improving the accuracy, although none are FDA approved or considered a reference standard for Chagas diagnosis. The radio-immunoprecipitation assay (T. cruzi RIPA) is a qualitative, more specific test for reactive antibodies to epimastigote lysates, and is employed routinely as a confirmatory test by some blood banks [Tobler L H et al., (2007) Transfusion 47: 90-96.]. Other assays, for example, the ESA (ELISA strip assay) [Cheng K Y et al., (2007) Clinical and Vaccine Immunology 14: 355-361], the Architect Chagas kit [Praast G et al., (2011) Diagnostic Microbiology and Infectious Disease 69: 74-81.], and the assay of Granjon et al. (2016), utilize recombinant antigens from T. cruzi. It is recognized that the complex proteome and life cycle of T. cruzi necessitates discovery of additional antigens [De Pablos L M and Osuna A (2012) Infection and Immunity 80: 2258-2264.] The diversity of human immune responses to the T. Cruzii infection [Chatelain E (2017)] also testifies to the need for employing many targets to accurately determine positivity within any large intended use population, especially those with asymptomatic disease. A need has been demonstrated for new validated markers and new approaches for measuring T. cruzi infection status and monitoring disease activity [Pinazo M J et al., (2013); Pinazo M J et al., (2014) Expert Rev Anti Infect Ther 12: 479-496.]

A pre-requisite for establishing the desired tests is to develop a single, robust platform that can accurately and reproducibly detect Chagas in a diverse, asymptomatic population such as blood donors. Additionally, a single test is desired to could simultaneously diagnose Chagas and other disease infections including infections caused by other pathogens e.g. West Nile Virus (WNV), that are endemic to the same geographical areas as T. cruzi. For blood banks this would also include viruses such as hepatitis B (HBV) and hepatitis C (HCV).

Current blood-testing laboratories use a separate series of assays, each performed on all blood samples along with the Chagas series, to ensure US transfusion recipients of infectious disease-free products [McCullough J (1993) JAMA 269: 2239-2245]. In addition to serologic screening, tests for the different viral series include nucleic acid screening based on a pooling and partitioning protocol [Busch M P et al., (2008) J Infect Dis 198: 984-993].

Similarly to the cases of Chagas disease, many subjects infected with the hepatitis B and hepatitis C viruses have no symptoms during the initial infection, and many who develop chronic disease remain asymptomatic. Additionally, viral hepatitis symptoms, when present, are similar no matter which hepatitis. Over the years, the infection often leads to liver disease and cirrhosis, which in turn can develop complications such as liver failure and liver cancer. Assays for the detection of HBV and HCV infection involve test that detect viral antigens or antibodies produced by the host. However, interpretation of these assays is complex. Furthermore, testing for HBV and HCV is not routinely performed, and development of serious complications in the host and transmission of the virus remain unchecked.

Similarly, the mosquito-borne infection caused by the West Nile virus may not produce any symptoms in approximately 80% of humans. If untreated, neurological disease including West Nile encephalitis, West Nile meningitis, WN meningoencephalitis, and WN poliomyelitis can develop. A number of various diseases may present with symptoms similar to those caused by a clinical WNV infection, e.g. enterovirus infection and bacterial meningitis. Accounting for differential diagnoses is crucial in the definitive diagnosis of WNV, and diagnostic and serologic tests including PCR and viral cultures are necessary to identify the specific pathogen causing the symptoms.

### Samples

The samples that are utilized according to the methods provided can be any biological samples. For example, the biological sample can be a biological liquid sample that comprises antibodies. Suitable biological liquid samples include, but are not limited to blood, plasma, serum, sweat, tears, sputum, urine, stool water, ear flow, lymph, saliva, cerebrospinal fluid, ravages, bone marrow suspension, vaginal flow, transcervical lavage, synovial fluid, aqueous humor, amniotic fluid, cerumen, breast milk, broncheoalveolar lavage fluid, brain fluid, cyst fluid, pleural and peritoneal fluid, pericardial fluid, ascites, milk, pancreatic juice, secretions of the respiratory, intestinal and genitourinary tracts, amniotic fluid, milk, and leukophoresis samples. A biological sample may also include the blastocyl cavity, umbilical cord blood, or maternal circulation which may be of fetal or maternal origin. In some embodiments, the sample is a sample that is easily obtainable by non-invasive procedures e.g. blood, plasma, serum, sweat, tears, sputum, urine, sputum, ear flow, or saliva. In certain embodiments the sample is a peripheral blood sample, or the plasma or serum fractions of a peripheral blood sample. As used herein, the terms “blood,” “plasma” and “serum” expressly encompass fractions or processed portions thereof.

Because of its minimally invasive accessibility and its ready availability, blood is the most preferred and used human body fluid to be measured in routine clinical practice. Moreover, blood perfuses all body tissues and its composition is therefore relevant as an indicator of the over-all physiology of an individual. In some embodiments, the biological sample that is used to obtain an immunosignature/antibody binding profile is a blood sample. In other embodiments, the biological sample is a plasma sample. In yet other embodiments, the biological sample is a serum sample. In yet other embodiments, the biological sample is a dried blood sample. The biological sample may be obtained through a third party, such as a party not performing the analysis of the antibody binding profiles, and/or the party performing the binding assay to the peptide array. For example, the sample may be obtained through a clinician, physician, or other health care manager of a subject from which the sample is derived. Alternatively, the biological sample may be obtained by the party performing the binding assay of the sample to a peptide array, and/or the same party analyzing the antibody binding profile/IS. Biological samples that are to be assayed, can be archived (e.g., frozen) or otherwise stored in under preservative conditions.

The terms “patient sample” and “subject sample” are used interchangeably herein to refer to a sample e.g. a biological sample, obtained from a patient i.e. a recipient of medical attention, care or treatment. The subject sample can be any of the samples described herein. In certain embodiments, the subject sample is obtained by non-invasive procedures e.g. peripheral blood sample.

An antibody binding profile of circulating antibodies in a sample can be obtained according to the methods provided using limited quantities of sample. For example, peptides on the array can be contacted with a fraction of a milliliter of blood to obtain an antibody binding profile comprising a sufficient number of informative peptide-protein complexes to identify the health condition of the subject.

In some embodiments, the volume of biological sample that is needed to obtain an antibody binding profile is less than 10 ml, less than 5 ml, less than 3 ml, less than 2 ml, less than 1 ml, less than 900 ul, less than 800 ul, less than 700 ul, less than 600 ul, less than 500 ul, less than 400 ul, less than 300 ul, less than 200 ul, less than 100 ul, less than 50 ul, less than 40 ul, less than 30 ul, less than 20 ul, less than 10 ul, less than 1 ul, less than 900 nl, less than 800 nl, less than 700 nl, less than 600 nl, less than 500 nl, less than 400 nl, less than 300 nl, less than 200 nl, less than 100 nl, less than 50 nl, less than 40 nl, less than 30 nl, less than 20 nl, less than 10 nl, or less than 1 nl. In some embodiments, the biological fluid sample can be diluted several fold to obtain a antibody binding profile. For example, a biological sample obtained from a subject can be diluted at least by 2-fold, at least by 4-fold, at least by 8-fold, at least by 10-fold, at least by 15-fold, at least by 20-fold, at least by 30-fold, at least by 40-fold, at least by 50-fold, at least by 100-fold, at least by 200-fold, at least by 300-fold, at least by 400-fold, at least by 500-fold, at least by 600-fold, at least by 700-fold, at least by 800-fold, at least by 900-fold, at least by 1000-fold, at least by 5000-fold, or at least by 10,000-fold. Antibodies present in the diluted serum sample, and are considered significant to the health of the subject, because if antibodies remain present even in the diluted serum sample, they must reasonably have been present at relatively high amounts in the blood of the patient.

An example of detecting a disease in a subject according to the methods described herein is given in the Examples. The examples demonstrate that correct diagnosis of infection was provided using a mere 90 microliters of serum or of plasma.

### Treatments and Conditions

The methods and arrays of the invention provide methods, assays and devices for identifying discriminating peptides, which can be used for screening of infections, and identifying candidate biomarkers of the infections. The methods and arrays of the embodiments disclosed herein can be used, for example, for screening infections and/or identifying one or more candidate biomarkers for infections in a subject. A subject can be a human, a guinea pig, a dog, a cat, a horse, a mouse, a rabbit, and various other animals. A subject can be of any age, for example, a subject can be an infant, a toddler, a child, a pre-adolescent, an adolescent, an adult, or an elderly individual.

The arrays and methods of the invention can be used by a user. A plurality of users can use a method of the invention to identify and/or provide a treatment of a condition. A user can be, for example, a human who wishes to monitor one's own health. A user can be, for example, a health care provider. A health care provider can be, for example, a physician. In some embodiments, the user is a health care provider attending the subject. Non-limiting examples of physicians and health care providers that can be users of the invention can include, an anesthesiologist, a bariatric surgery specialist, a blood banking transfusion medicine specialist, a cardiac electrophysiologist, a cardiac surgeon, a cardiologist, a certified nursing assistant, a clinical cardiac electrophysiology specialist, a clinical neurophysiology specialist, a clinical nurse specialist, a colorectal surgeon, a critical care medicine specialist, a critical care surgery specialist, a dental hygienist, a dentist, a dermatologist, an emergency medical technician, an emergency medicine physician, a gastrointestinal surgeon, a hematologist, a hospice care and palliative medicine specialist, a homeopathic specialist, an infectious disease specialist, an internist, a maxillofacial surgeon, a medical assistant, a medical examiner, a medical geneticist, a medical oncologist, a midwife, a neonatal-perinatal specialist, a nephrologist, a neurologist, a neurosurgeon, a nuclear medicine specialist, a nurse, a nurse practioner, an obstetrician, an oncologist, an oral surgeon, an orthodontist, an orthopedic specialist, a pain management specialist, a pathologist, a pediatrician, a perfusionist, a periodontist, a plastic surgeon, a podiatrist, a proctologist, a prosthetic specialist, a psychiatrist, a pulmonologist, a radiologist, a surgeon, a thoracic specialist, a transplant specialist, a vascular specialist, a vascular surgeon, and a veterinarian. A diagnosis identified with an array and a method of the invention can be incorporated into a subject's medical record.

### Array Platform

In some embodiments, disclosed herein are methods and process that provide for array platforms that allow for increased diversity and fidelity of chemical library synthesis. The array platforms comprise a plurality of individual features on the surface of the array. Each feature typically comprises a plurality of individual molecules, which are optionally synthesized in situ on the surface of the array, wherein the molecules are identical within a feature, but the sequence or identity of the molecules differ between features. The array molecules include, but are not limited to nucleic acids (including DNA, RNA, nucleosides, nucleotides, structure analogs or combinations thereof), peptides, peptide-mimetics, and combinations thereof and the like, wherein the array molecules may comprise natural or non-natural monomers within the molecules. Such array molecules include the synthesis of large synthetic peptide arrays. In some embodiments, a molecule in an array is a mimotope, a molecule that mimics the structure of an epitope and is able to bind an epitope-elicited antibody. In some embodiments, a molecule in the array is a paratope or a paratope mimetic, comprising a site in the variable region of an antibody (or T cell receptor) that binds to an epitope an antigen. In some embodiments, an array of the invention is a peptide array comprising random, pseudo-random or maximally diverse peptide sequences.

The peptide arrays can include control sequences that match epitopes of well characterized monoclonal antibodies (mAbs). Binding patterns to control sequences and to library peptides can be measured to qualify the arrays and the immunosignature assay process, mAbs with known epitopes e.g. 4C1, p53Ab1, p53Ab8 and LnKB2, can be assayed at different doses. Additionally, inter wafer signal precision can be determined by testing sample replicates e.g. plasma samples, on arrays from different wafers and calculating the coefficients of variation (CV) for all library peptides. Precision of the measurements of binding signals can be determined as an aggregate of the inter-array, inter-slide, inter-wafer and inter-day variations made on arrays synthesized on wafers of the same batch (within wafer batches). Additionally, precision of measurements can be determined for arrays on wafers of different batches (between wafer batches). In some embodiments, measurements of binding signals can be made within and/or between wafer batches with a precision varying less than 5%, less than 10%, less than 15%, less than 20%, less than 25%, or less than 30%.

The technologies disclosed herein include a photolithographic array synthesis platform that merges semiconductor manufacturing processes and combinatorial chemical synthesis to produce array-based libraries on silicon wafers. By utilizing the tremendous advancements in photolithographic feature patterning, the array synthesis platform is highly-scalable and capable of producing combinatorial chemical libraries with 40 million features on an 8-inch wafer. Photolithographic array synthesis is performed using semiconductor wafer production equipment in a class 10,000 clean room to achieve high reproducibility. When the wafer is diced into standard microscope slide dimensions, each slide contains more than 3 million distinct chemical entities.

In some embodiments, arrays with chemical libraries produced by photolithographic technologies disclosed herein are used for immune-based diagnostic assays, for example called immunosignature assays. Using a patient's antibody repertoire from a drop of blood bound to the arrays, a fluorescence binding profile image of the bound array provides sufficient information to classify disease vs. healthy.

In some embodiments, immunosignature assays are being developed for clinical application to diagnose/monitor infectious diseases and to assess response to infectious treatments. Exemplary embodiments of immunosignature assays is described in detail in US Pre-Grant Publication No. 2012/0190574, entitled “Compound Arrays for Sample Profiling” and US Pre-Grant Publication No. 2014/0087963, entitled “Immunosignaturing: A Path to Early Diagnosis and Health Monitoring”, both of which are incorporated by reference herein for such disclosure. The arrays developed herein incorporate analytical measurement capability within each synthesized array using orthogonal analytical methods including ellipsometry, mass spectrometry and fluorescence. These measurements enable longitudinal qualitative and quantitative assessment of array synthesis performance.

In some embodiments, the array is a wafer-based, photolithographic, in situ peptide array produced using reusable masks and automation to obtain arrays of scalable numbers of combinatorial sequence peptides. In some embodiments, the peptide array comprises at least 5,000, at least 10,000, at least 15,000, at least 20,000, at least 30,000, at least 40,000, at least 50,000, at least 100,000, at least 200,000, at least 300,000, at least 400,000, at least 500,000, at least 1,000,000, at least 2,000,000, at least 3,000,000, at least 4,000,000, at least 5,000,000, at least 10,000,000, at least 100,000,000 or more peptides having different sequences. Multiple copies of each of the different sequence peptides can be situated on the wafer at addressable locations known as features.

In some embodiments, detection of antibody binding on a peptide array poses some challenges that can be addressed by the technologies disclosed herein. Accordingly, in some embodiments, the arrays and methods disclosed herein utilize specific coatings and functional group densities on the surface of the array that can tune the desired properties necessary for performing immunosignature assays. For example, non-specific antibody binding on a peptide array may be minimized by coating the silicon surface with a moderately hydrophilic monolayer polyethylene glycol (PEG), polyvinyl alcohol, carboxymethyl dextran, and combinations thereof. In some embodiments, the hydrophilic monolayer is homogeneous. Second, synthesized peptides are linked to the silicon surface using a spacer that moves the peptide away from the surface so that the peptide is presented to the antibody in an unhindered orientation.

The in situ synthesized peptide libraries are disease agnostic and can be synthesized without a priori awareness of a disease they are intended to diagnose. Identical arrays can be used to determine any health condition.

The term “peptide” as used herein refers to a plurality of amino acids joined together in a linear or circular chain. For purposes of the present invention, the term peptide is not limited to any particular number of amino acids. Preferably, however, they contain up to about 400 amino acids, up to about 300 amino acids, up to about 250 amino acids, up to about 150 amino acids, up to about 70 amino acids, up to about 50 amino acids, up to about 40 amino acids, up to 30 amino acids, up to 20 amino acids, up to 15 amino acids, up to 10 amino acids, or up to 5 amino acids. In some embodiments, the peptides of the array are between 5 and 30 amino acids, between 5 and 20 amino acids, or between 5 and 15 amino acids. The amino acids forming all or a part of a peptide molecule may be any of the twenty conventional, naturally occurring amino acids, i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y). Any of the amino acids in the peptides forming the present arrays may be replaced by a non-conventional amino acid. In general, conservative replacements are preferred. In some embodiments, the peptides on the array are synthesized from less of the 20 amino acids. In some embodiments, one or more of amino acids methionine, cysteine, isoleucine and threonine are excluded during synthesis of the peptides.

### Digital Processing Device

In some embodiments, the systems, platforms, software, networks, and methods described herein include a digital processing device, or use of the same. In further embodiments, the digital processing device includes one or more hardware central processing units (CPUs), i.e., processors that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, a digital processing device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, a digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, a digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

In some embodiments, a digital processing device includes a digital camera. In some embodiments, a digital camera captures digital images. In some embodiments, the digital camera is an autofocus camera. In some embodiments, a digital camera is a charge-coupled device (CCD) camera. In further embodiments, a digital camera is a CCD video camera. In other embodiments, a digital camera is a complementary metal-oxide-semiconductor (CMOS) camera. In some embodiments, a digital camera captures still images. In other embodiments, a digital camera captures video images. In various embodiments, suitable digital cameras include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and higher megapixel cameras, including increments therein. In some embodiments, a digital camera is a standard definition camera. In other embodiments, a digital camera is an HD video camera. In further embodiments, an HD video camera captures images with at least about 1280× about 720 pixels or at least about 1920× about 1080 pixels. In some embodiments, a digital camera captures color digital images. In other embodiments, a digital camera captures grayscale digital images. In various embodiments, digital images are stored in any suitable digital image format. Suitable digital image formats include, by way of non-limiting examples, Joint Photographic Experts Group (JPEG), JPEG 2000, Exchangeable image file format (Exif), Tagged Image File Format (TIFF), RAW, Portable Network Graphics (PNG), Graphics Interchange Format (GIF), Windows® bitmap (BMP), portable pixmap (PPM), portable graymap (PGM), portable bitmap file format (PBM), and WebP. In various embodiments, digital images are stored in any suitable digital video format. Suitable digital video formats include, by way of non-limiting examples, AVI, MPEG, Apple® QuickTime®, MP4, AVCHD®, Windows Media®, DivX™, Flash Video, Ogg Theora, WebM, and RealMedia.

### Non-Transitory Computer Readable Storage Medium

In some embodiments, the systems, platforms, software, networks, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the systems, platforms, software, networks, and methods disclosed herein include at least one computer program. A computer program includes a sequence of instructions, executable in the digital processing device's CPU, written to perform a specified task. In light of the disclosure provided herein, those of skill in the art will recognize that a computer program may be written in various versions of various languages. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. A web application for providing a career development network for artists that allows artists to upload information and media files, in some embodiments, includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

The systems, platforms, software, networks, and methods disclosed herein include, in various embodiments, software, server, and database modules. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

The present invention is described in further detail in the following Examples which are not in any way intended to limit the scope of the invention as claimed. The attached Figures are meant to be considered as integral parts of the specification and description of the invention. The following examples are offered to illustrate, but not to limit the claimed invention.

### EXAMPLES

### Example 1—Immunosignature Methods for the Diagnosis of Infections

Immunosignature assays were developed to detect and differentiate T. cruzii, HBV, HCV, and WNV infections according to the following.

Donor Samples.

Donor plasma samples serologically positive for Chagas antibodies, along with age and gender matched healthy donor plasma, and plasma samples that tested seropositive for hepatitis B virus (HBV), hepatitis C virus (HCV) or West Nile virus (WNV) (WNV), were obtained from Creative Testing Solutions (Tempe, Ariz.). Two cohorts of samples were obtained, one in 2015 and a second set in 2016. Upon receipt, the plasma was thawed, mixed 1:1 with ethylene glycol as a cryoprotectant and aliquoted into single use volumes. Single use aliquots were stored at −20° C. until needed. The remaining sample volume was stored neat at −80° C. Identities of all samples were tracked using 2D barcoded tubes (Micronic, Leystad, the Netherlands). In preparation for assay, sample aliquots were warmed on ice to 4° C. and diluted 1:100 in primary incubation buffer (Phosphate Buffered Saline with 0.05% Tween 20 (PBST) and 1% mannitol). Microtiter plates containing the 1:100 dilutions were then diluted to 1:625 for use in the assay. For the subset of samples selected for evaluating platform performance across wafer lots, the 1:100 dilutions were aliquoted into single use microtiter plates and stored at −80° C. All aliquoting and dilution steps were performed using a BRAVO robotic pipetting station (Agilent, Santa Clara, Calif.). All procedures using de-identified, banked samples were reviewed by the Western Institutional Review Board (protocol no. 20152816).

Arrays. A combinatorial library of 126,009 peptides with a median length of 9 residues and range from 5 to 13 amino acids was designed to include 99.9% of all possible 4-mers and 48.3% of all possible 5-mers of 16 amino acids (methionine, M; cysteine, C; isoleucine, I; and threonine, T were excluded). These were synthesized on an 200 mm silicon oxide wafer using standard semiconductor photolithography tools adapted for tert-butyloxycarbonyl (BOC) protecting group peptide chemistry (Legutki J B et al., Nature Communications. 2014; 5:4785). Briefly, an aminosilane functionalized wafer was coated with BOC-glycine. Next, photoresist containing a photoacid generator, which is activated by UV light, was applied to the wafer by spin coating. Exposure of the wafer to UV light (365 nm) through a photomask allows for the fixed selection of which features on the wafer will be exposed using a given mask. After exposure to UV light, the wafer was heated, allowing for BOC-deprotection of the exposed features. Subsequent washing, followed the by application of an activated amino acids completes the cycle. With each cycle, a specific amino acid was added to the N-terminus of peptides located at specific locations on the array. These cycles were repeated, varying the mask and amino acids coupled, to achieve the combinatorial peptide library. Thirteen rectangular regions with the dimensions of standard microscope slides, were diced from each wafer. Each completed wafer was diced into 13 rectangular regions with the dimensions of standard microscope slides (25 mm×75 mm). Each of these slides contained 24 arrays in eight rows by three columns. Finally, protecting groups on the side chains of some amino acids were removed using a standard cocktail. The finished slides were stored in a dry nitrogen environment until needed. A number of quality tests are performed ensure arrays are manufactured within process specifications including the use of 3σ statistical limits for each step. Wafer batches are sampled intermittently by MALDI-MS to identify that each amino acid was coupled at the correct step, ensuring that the individual steps constituting the combinatorial synthesis are correct. Wafer manufacturing is tracked from beginning to end via an electronic custom Relational Database which is written in Visual Basic and has an access front end with an SQL back end. The front-end user interface allows operators to enter production info into the database with ease. The SQL backend allows us a simple method for database backup and integration with other computer systems for data share as needed. Data typically tracked include chemicals, recipes, time and technician performing tasks. After a wafer is produced the data is reviewed and the records are locked and stored. Finally, each lot is evaluated in a binding assay to confirm performance, as described below.

Plasma Assay.

Production quality manufactured microarrays were obtained and rehydrated prior to use by soaking with gentle agitation in distilled water for 1 h, PBS for 30 min and primary incubation buffer (PBST, 1% mannitol) for 1 h. Slides were loaded into an ArrayIt microarray cassette (ArrayIt, Sunnyvale, Calif.) to adapt the individual microarrays to a microtiter plate footprint. Using a liquid handler, 90 μl of each sample was prepared at a 1:625 dilution in primary incubation buffer (PBST, 1% mannitol) and then transferred to the cassette. This mixture was incubated on the arrays for 1 h at 37° C. with mixing on a TeleShake95 (INHECO, Martinsried, Germany) to drive antibody-peptide binding. Following incubation, the cassette was washed 3× in PBST using a BioTek 405TS (BioTek, Winooski, Vt.). Bound antibody was detected using 4.0 nM goat anti-human IgG (H+L) conjugated to AlexaFluor 555 (Thermo-Invitrogen, Carlsbad, Calif.), or 4.0 nM goat anti-human IgA comjugated to DyLight 550 (Novus Biologicals, Littleton, Colo.) in secondary incubation buffer (0.5% casein in PBST) for 1 h with mixing on a TeleShake95 platform mixer, at 37° C. Following incubation with secondary, the slides were again washed with PBST followed by distilled water, removed from the cassette, sprayed with isopropanol and centrifuged dry. Quantitative signal measurements were obtained by determining a relative fluorescent value for each addressable peptide feature. Separately, ELISAs were conducted to assess cross-reactivity between the anti-IgG and anti-IgA secondary antibody products. A low level of cross-reactivity was noted for the anti-IgG product against an IgA monoclonal; no reactivity was found for the anti IgA product against an IgG monoclonal.

Monoclonal Assay.

Prior to conducting the IST assays with donor plasma, the binding activity of commercial, murine monoclonal antibodies (mAb) to control peptides, corresponding to each mAb's established epitope sequence, was evaluated. The IST arrays were probed in triplicate with 2.0 nM each of antibody clones 4C1 (Genway), p53Ab1 (Mllipore), p53Ab8 (Millipore), and LnkB2 (Absolute Antibody) in primary incubation buffer (l % mannitol, PBST). Secondary incubation and quantification of signal were the same as described above.

Data Acquisition.

Assayed microarrays were imaged using an Innopsys 910AL microarray scanner fitted with a 532 nm laser and 572 nm BP 34 filter (Innopsys, Carbonne, France). The Mapix software application (version 7.2.1) identified regions of the images associated with each peptide feature using an automated gridding algorithm. Median pixel intensities for each peptide feature were saved as a tab-delimitated text file and stored in a database for analysis.

Data Analysis.

The median feature intensities were log₁₀ transformed after adding a constant value of 100 to improve homoscedasticity. The intensities on each array were normalized by subtracting the median intensity of the combinatorial library features for that array.

In the monoclonal assays, selective binding of each monoclonal to its cognate epitope was assessed using a Z-score, calculated as:

$$Z = \frac{mean ( {I}_{mAb} ) - mean ( {I}_{2 °} )}{sd ( {I}_{2 °} )}$$

where I_{mAb} and I₂ₒ are the transformed peptide intensities in the presence of monoclonal or secondary antibody only, respectively. Binding to each of the peptides containing an epitope of one of the mAbs was measured on all four mAbs.

In the IST assays, binding of plasma antibodies to each feature was measured by quantifying fluorescent signal. Peptide features that showed differential signal between groups were determined by t-test of mean peptide intensities with the Welch adjustment for unequal variances. For the 2105 Chagas cohort, Chagas seropositive donors (n=146) were compared to seronegative donors (n=189), and peptides with significantly differential signal were identified. A second set of peptides that could discriminate Chagas from other infectious diseases was identified by comparing mean intensities among Chagas seropositive donors (n=88) to Chagas seronegative donors who were positive for HCV (n=71), HBV (n=88) or WNV (n=88) by standard blood panel testing algorithms. Peptides that showed significant discrimination were identified based on 5% threshold for false positives after applying the Bonferroni correction for multiplicity (i.e., p<4e-7). In addition, a Pearson correlation was calculated for the transformed peptide intensities of Chagas-positive donors to their median signal over cut-off value (S/CO) from three T. cruzi ELISA assays. Also, peptides correlated to S/CO were identified using a 10% false discovery rate criterion by the Benjamini-Hochberg method (Benjamini Y and Hochberg Y [1995]Journal of the Royal Statistical Society, Series B 57: 289-300) within the 2015 cohort.

To construct a classifier, features were ranked for their ability to discriminate Chagas positive from other samples based on the p value associated with a Welch's t-test comparing Chagas positive to Chagas negative donors, or between the different disease types in the multi-disease model. The number of peptides selected was varied between 5 and 4000 features in steps and each of the selected features was input to a support vector machine (Cortes C, and Vapnik V. Machine Learning. 1995; 20(3):273-97) with a linear kernel and cost parameter of 0.01 to train a classifier. A four-fold or five-fold cross validation repeated 100 times was used to quantify model performance, estimated as the error under the receiver-operating characteristic curve (AUC), and incorporated both feature selection and classifier development to avoid bias.

Finally, a fixed SVM classifier was fit in the 2015 cohort using the optimal number of features based on performance under cross-validation, selected by their t-test p-values. This model was used in assessing precision and reproducibility of the platform, and was also evaluated in the 2016 cohort as an independent verification test of the cross-validation analyses.

All analyses were performed using R version 3.2.5. (Team RC. R: A language and environment for statistical computing. R Foundation for Statistical Computing Vienna 2016. Available from: https://www.R-project.org/.)

Peptide Alignment Scoring.

Library peptides were aligned to the T. cruzi CL Bener proteome [Sodre C L et al., (2009) Arch Microbiol 191: 177-184]. The alignment algorithm used a modified BLAST strategy [Altschul SF and Gish W (1996) Methods Enzymol 266: 460-480], requiring a seed of 3 amino acids, a gap penalty of 4 amino acids, and a scoring matrix of BLOSUM62 [Henikoff and, Henikoff J G (1992) Proc Natl Acad Sci USA 89: 10915-10919] modified to reflect the amino acids composition of the array [States D J et al., (1991) Methods 3: 66-70]. These modifications increase the score of similar substitutions, remove penalties for amino acids absent from the array and score all exact matches equally.

To generate an alignment score to a protein for a set of classifying library peptides i.e. discriminating peptides, those that yield a positive BLAST score are assembled into a matrix, with each row of the matrix corresponding to an aligned peptide and each column corresponding to one of the amino acids in the protein's sequence. Gaps and deletions are permitted within the peptide rows for alignment to the protein. In this way, each position in the matrix receives a score associated with the aligned amino acid of the peptide and protein. Each column, corresponding to an amino acid in the protein, is then summed to create an overlap score; this represents coverage of that amino acids position by the classifying peptides. To correct this score for library composition, another overlap score is calculated using an identical method for a list of all array peptides. This allows for the calculation of a peptide overlap difference score, s, at each amino acids position via the equation:

s_{d}=a−(b/d)*c

In this equation, a is the overlap score from the discriminating peptides, b is the number of discriminating peptides, c is the overlap score for the full library of peptides and d is the number of peptides in the library.

To convert these s scores (which were at the amino acids level) to a full-protein statistic, the sum of scores for every possible tiling 20-mer epitope within a protein is calculated. The final protein score, also known as protein epitope score, S_{d} is the maximum along this rolling window of 20 for each protein. A similar set of scores was calculated for 100 iterative-rounds of randomly selecting peptides from the library, equal in number to the number of discriminating peptides. The p-value for each score, S, is calculated based on the number of times this score is met or exceeded among the randomly selected peptides, controlling for the number of iterations.

Precision, Reproducibility and Performance Analyses.

The precision of antibody binding to the array features was characterized for a set of eight plasma samples by measuring the signals of 200 peptides used in a Chagas fixed classifier model. Four Chagas seropositive donors displaying a range of S/CO values and three Chagas seronegative samples were selected from the full cohort of donors. These were assayed in triplicate. A well-characterized in-house plasma sample from a healthy donor was also included in the slide design, assayed in duplicate. As a negative control, one array was incubated without plasma in the primary incubation step but incubated with the secondary detection antibody. These 24 samples were distributed evenly across the array positions on a single slide. This slide layout was then replicated across multiple slides.

To evaluate precision within a batch, three wafers from a single manufacturing lot were selected. Twelve of the thirteen slides from each wafer were evaluated using the one-slide precision design described above. The slides were evaluated across three ArrayIt cassettes per day on three different days. Slides from each wafer were assigned evenly across the three days such that each cassette contained two slides from one of the three wafers and one slide each from the remaining two wafers.

To measure precision between batches one wafer from each of four different production lots was selected. Twelve of the thirteen slides from each wafer were evaluated using the precision study sample-set described above. These slides were distributed for testing across four cassettes per day, spanning three days. Slides from each wafer were distributed evenly across the 3 days such that each cassette contained two slides from two of the four wafers. A mixed effects model was used to estimate the sources of experimental variance. Donor sample was treated as a fixed effect. The nested factors ‘wafer’, ‘slide’, and ‘array’ were crossed with ‘day’, and these were treated as random effects. Models were fit in R using the Ime4 package to derive coefficients of variance (CV).

To assess the robustness of the ImmunoSignature classifier across many wafer manufacturing batches and assays, a quality control (QC) sample-set was selected that could be assayed on a single slide. It was comprised of a representative panel of 11 cases and 11 controls that were assayed on a single slide from 22 different wafers manufactured across 10 synthesis batches. For each of the 22 wafer-slides tested, the fixed model classifier developed in the Chagas trial was applied to this sample set to estimate area under the receiver operator characteristic (ROC) curve. One of these wafers was used for the Chagas trial and another for the mixed cohort (Chagas, HBV, HCV, & WNV) trial.

### Example 2—Platform Validation

Experiments were conducted using monoclonal antibodies to evaluate the quality of final in situ synthesized array peptide products with respect to ligand presentation and antibody recognition.

All diagnostic assays were conducted on a validated microarray platform.

A peptide synthesis protocol was developed in which parallel coupling reactions are performed directly on silicon wafers using masks and photolithographic techniques. Arrays displaying a total of 131,712 peptides (median length of 9 amino acids) at features of 14 μm×14 μm each were utilized to query antibody-binding events. The array layout included 126,009 library-peptide features and 6203 control-peptide features attached to the surface via a common linker (see Example 1). The library peptides were designed to evenly sample all possible amino acids combinations. The control peptides include 500 features that correspond to the established epitopes of five different well-characterized monoclonal antibodies (mAb), each replicated 100 times. Another 935 features correspond to four different sequence variants of three of the five epitopes, each replicated from 100 to 280 times. An additional 500 control features were designed with amino acids compositions similar to those of the library peptides, but are uniformly 8-mers and present in triplicate. The median signals of these 500 control features were quantitated and treated as part the library when developing the IST models. The remaining 3,268 controls include fiducial markers to aid grid alignment, analytic control sequences and linker-only features. Aside from the fiducials, all features are distributed evenly across the array.

Experiments were conducted using mAbs that evaluated the quality of final array-synthesized products with respect to ligand presentation and antibody recognition. A panel of four murine antibody clones: 4C1, p53Ab1, p53 Ab8, and LnkB2 were selected with recognition sequences that correspond to four of the five control epitopes designed within the array layout. The sequence contents of the four array-represented epitopes collectively include all 16 amino acids that were used to build the library.

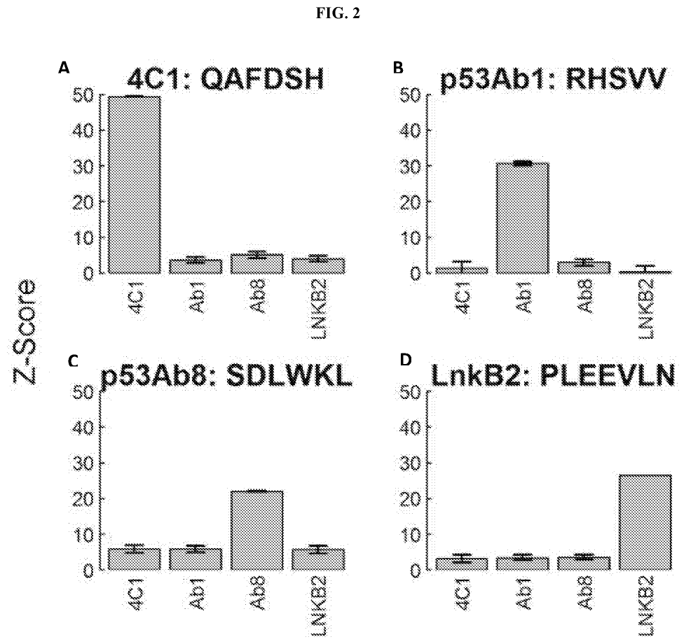

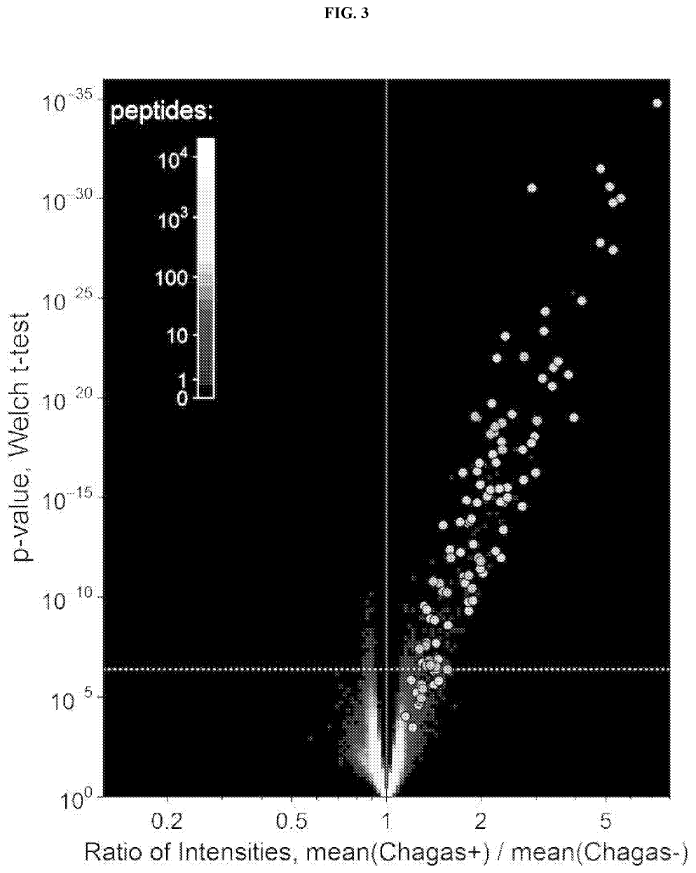

FIG. 2 presents the results from a binding assay conducted as described (see Example 1) in which each antibody was individually applied to an array with competitor agent, in triplicate. For each mAb, the control feature intensities were used to calculate a Z score for both the peptide sequence corresponding to its epitope, and the three non-cognate sequences. Each of the cognate sequences were bound with high signal intensity whereas the non-cognates displayed little or no signal above background values (secondary only).

These data validate the integrity of the synthetic library products. The data indicate that the microarrays carry peptides suitable for specific antibody recognition and binding. The use of photolithography and masks for the in situ process provides an opportunity for production scaling and efficient costing. Notably, the exact same library array design can be used to identify peptides that distinguish a variety of different conditions e.g. infections, as is exemplified by the accuracy of classification of Chagas disease, HPV, HCV, and WNV (Tables 4 and 5).

### Example 3—Immunosignature Assay Differentiates Subjects that are Seropositive for T. crui from Subjects that are Seronegative for T. Cruzi

Two cohorts of plasma samples of asymptomatic donors were obtained from a blood bank repository (Creative Testing Solutions, Tempe, Ariz.), and are shown in Table 1. The 2015 cohort is of 335 donors that were each serologically tested for Chagas disease using the blood bank's algorithm. The testing is intended to prevent entry of samples into the blood supply from any donor with indications of Chagas. First, three ELISAs were serially performed that assayed plasma against whole T. cruzi lysate (Ortho). If any one of these is scored positive by a signal to cutoff value (S/CO>1.0), then a confirmatory test is performed. This is an immunoprecipitation assay (T cruzi RIPA) that uses the plasma to precipitate radiolabeled T cruzi lysates. By these criteria 189 donors were seropositive and 146 were seronegative. An S/CO score of >4.0 is considered to be strong positivity [Remesar M et al., (2015) Transfusion 55: 2499-2504], which places 49 (26%) seropositive donors into this high S/CO subgroup. The distributions of gender, age, and ethnicity were those typically observed in a US blood donor population. The 2016 cohort is of 116 donors that were tested for Chagas with the same protocol of serial ELISA and RIPA testing described above. The results identified 58 Chagas seropositive and 58 seronegative participants. A higher proportion of the Chagas positive individuals (31 of 58 (53%) scored into the high S/CO>4 subgroup. The distributions of gender and age are similar although ethnicity was mildly skewed in this second donor population.

TABLE 1
Description of donors in the Chagas Study

| | Training cohort (2015) | | | | Test cohort (2016) | | | |
|---|---|---|---|---|---|---|---|---|
| | | Chagas | Chagas | | | Chagas | Chagas | |
| | all | neg | pos | S/CO > 4 | all | neg | pos | S/CO > 4 |
| Group size | 335 | 189 | 146 | 49 | 116 | 58 | 58 | 31 |
| Gender | | | | | | | | |
| female | 90 | 80 | 10 | 2 | 48 | 24 | 24 | 11 |
| male | 127 | 109 | 18 | 6 | 68 | 34 | 34 | 20 |
| unknown | 118 | 0 | 118 | 41 | 0 | 0 | 0 | 0 |
| Ethnicity | | | | | | | | |
| white | 145 | 144 | 1 | 1 | 14 | 8 | 6 | 4 |
| Hispanic | 49 | 32 | 17 | 4 | 84 | 43 | 41 | 24 |
| black | 4 | 4 | 0 | 0 | 3 | 2 | 1 | 0 |
| other | 10 | 9 | 1 | 0 | 2 | 2 | 0 | 0 |
| unknown | 127 | 0 | 127 | 44 | 13 | 3 | 10 | 3 |
| Age bin | | | | | | | | |
| (15-20) | 10 | 9 | 1 | 1 | 16 | 7 | 9 | 5 |
| (20-30) | 29 | 26 | 3 | 0 | 20 | 11 | 9 | 5 |
| (30-40) | 52 | 46 | 6 | 1 | 24 | 14 | 10 | 6 |
| (40-50) | 38 | 33 | 5 | 2 | 26 | 9 | 17 | 7 |
| (50-60) | 38 | 32 | 6 | 1 | 21 | 11 | 10 | 7 |
| (60-70) | 29 | 26 | 3 | 2 | 7 | 4 | 3 | 1 |
| (70-87) | 21 | 17 | 4 | 1 | 2 | 2 | 0 | 0 |
| unknown | 118 | 0 | 118 | 41 | 0 | 0 | 0 | 0 |

The study trial presented here was conducted by using the 2015 cohort as an algorithm-training set to develop a classifier that distinguishes Chagas seropositive from seronegative individuals. This classifier was fixed and then applied to predict the positivity of the 2016 cohort donors. Thus, the 2016 samples represented a training-independent verification set.

### Evaluating the Performance of the Immunosignature for Determining Chagas Positivity

Immunosignature (IST) assays were performed as described in Example 1 and scanned to acquir signal intensity measurements at each feature. Application of Welch's t-test identified 356 individual peptides that had significant differences in mean signal between those donors who were blood-bank scored as seropositive versus seronegative for Chagas. As demarcated in FIG. 3 by a white dotted line, most, but not all, of the significantly distinguishing peptides displayed higher binding intensities in the Chagas positive as compared to Chagas negative donors. Many of these peptides had signals that were also positively correlated to the median T. cruzi S/CO value of all Chagas positive donors (shown as blue and green circles). This is consistent with the possibility that some library peptides may bind the same or related plasma-antibodies as those bound by antigen in the ELISA screen. There were 14 peptides that are significantly correlated to S/CO but did not meet the Bonferroni threshold for IST discrimination of Chagas positivity (circles below white dashed line). Notably, many of the 356 peptides that showed the strongest discrimination by IST were not significantly correlated to S/CO values. This demonstrates that the binding data collected by IST (t-test) shares some overlap with that collected by ELISA (S/CO) but indicates that unique interactions were also measured.

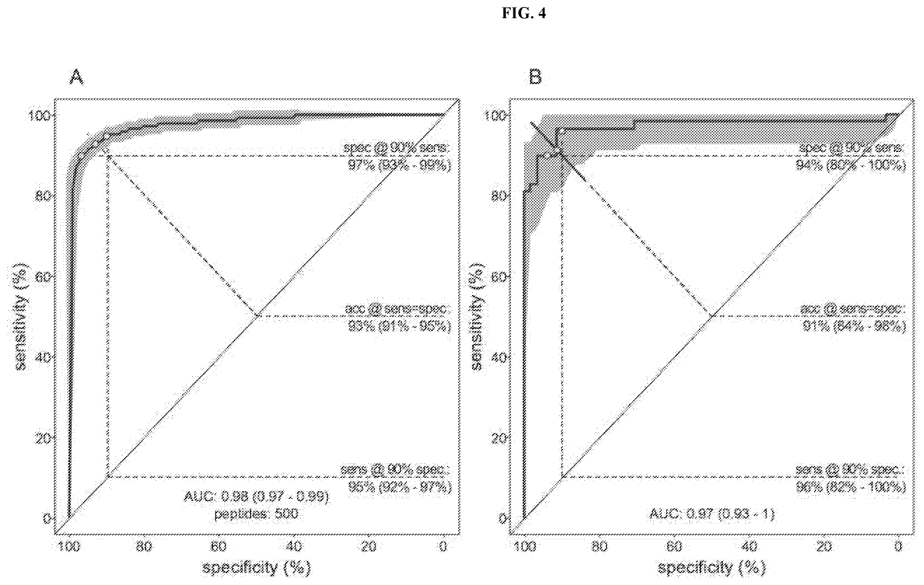

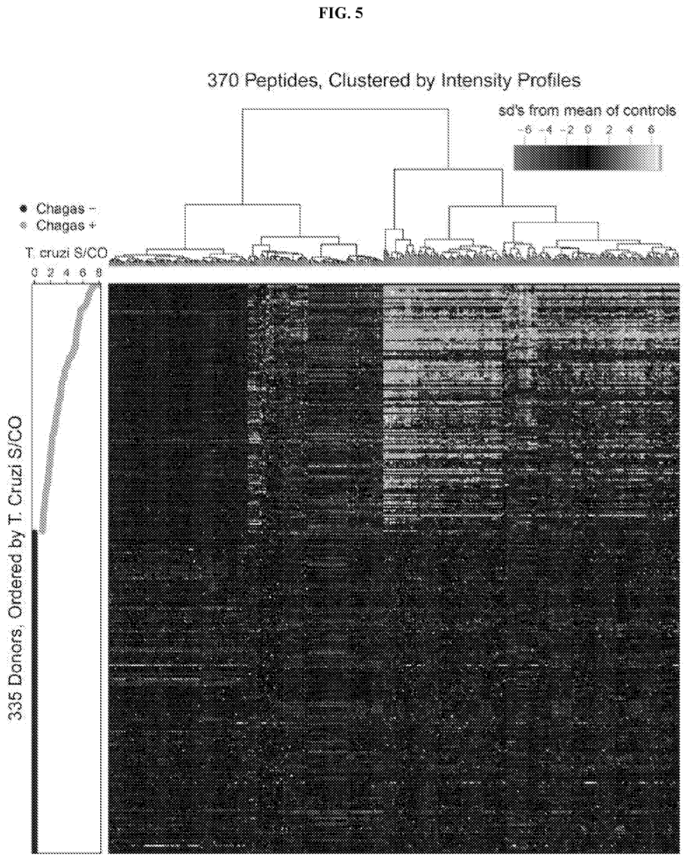

A support vector machine (SVM) classifier of Chagas seropositivity was developed in the 2015 cohort. Under cross-validation, the best performance was achieved when the top 500 peptides, as ranked by Welch t-test were input to the model. This number is greater than 356 that met the Bonferroni significance cutoff, indicating that additional information content existed in some of the peptides meeting the less stringent, false discovery rate (FDR) cutoff of significance. FIG. 4A shows the relationship between mean sensitivity and specificity of 100 iterations of five-fold cross validation models, using the top 500 peptides within each training sample, as a function of diagnostic threshold. The area under the curve (AUC) estimates that for a donor chosen at random from within each of the two groups, the seropositive donor would have a 98% probability of being classified with a higher likelihood of Chagas positivity than the seronegative donor, with a 95% confidence interval (CI) of 97%-99%. At the threshold where sensitivity equaled specificity, the accuracy was 93% (CI=91%-95%). The cross-validation estimates were confirmed by application of a single, fixed SVM classifier using the top 500 peptides to the 2016 cohort, where the performance observed (AUC 97%; accuracy 91%) was within the 95% CI of the cross-validation estimates (FIG. 4B).

This same fixed classifier was used to assess the binding precision and reproducibility of the assay using a protocol in which four Chagas seropositive donors and three Chagas seronegative samples were repeatedly assayed as described in the Methods section. Classification accuracy was repeatedly calculated. These precision measurements indicated the following binding signal CVs for the IST assay features which comprise the fixed classifier: inter-array=11%, inter-slide=4%, inter-wafer=2.7%, inter-day=7.7%, and inter-batch=14.6%. Reproducibility of classification was also determined, as described in the Methods, indicating AUCs>0.98 (median AUC=1.0).

The results in FIG. 5 explore the heterogeneity of antibody binding across the 2015 Chagas cohort. The relative signal intensities are displayed for the 370 (356+14) peptides described in FIG. 3 that provided significant discrimination of Chagas positivity by t-test, by correlation to the ELISA S/CO levels or both criteria (FIG. 21 A-N).

The peptides that discriminated Chagas seropositive from Chagas seronegative samples were found to be enriched by greater than 100% in one or more motifs listed in FIG. 9B-F relative to the incidence of the same motifs in the entire peptide library. Additionally, 99% of the peptides that discriminated seropositive from seronegative samples were found to be enriched by greater than 100% in one or more amino acids arginine, aspartic acid, and lysine (FIG. 9A).

Each peptide (x axis) for each donor (y axis) is represented, and is shaded relative to the difference in its intensity compared to the mean intensity of the same peptide in all seronegative donors, which serve as controls. The heatmap color scheme is scaled by the standard deviation (sd) of a feature's signal from that of the controls. The legend has been truncated at 7 sd's to permit smaller, but significant variations to be visualized. The donors were ordered by their median reported ELISA S/CO measurements, and these data are plotted alongside the heatmap. The peptides have been clustered as indicated by the dendrogram at the top. The distinction between ELISA positive and negative donors is evident in the heatmap visualization, as are correlations between some peptides' IST signals and the ELISA signal levels. The Chagas positive samples display at least three distinct binding profiles for a subset of the peptides with i) uniformly lower signal than controls, ii) marginally higher signal than controls and iii) signal that increases as S/CO value increases. Peptide signal heterogeneity in the Chagas negative samples is relatively minor.

These data indicate that the different clusters may correlate with the status of the infection, and/or indicate disease progression.

In addition to measuring the IgG antibodies bound to the IST peptide array, IgA binding activity was determined, by simply detecting the plasma-antibody binding-events with a fluorescently-labeled anti-IgA specific secondary reagent. Fewer library peptides (224) passed the Bonferroni cutoff for significantly different signal levels between the seropositive and negative donors, and these overlapped with 50% of those detected by the anti-IgG secondary reagent. Additionally, all 23 IgA-classifying peptides that correlated to S/CO values were found within the list of 26 IgG-classifying peptides that correlated with S/CO (23/26=88% overlap). The performance of the IgA classification (AUC=0.94) was similar to that of the IgG classifier.

These findings indicate that a correlation exists between the IST test results and the disease-specific immune activity. These findings suggest the use of the immunosignature method as a test for monitoring the status of the T. cruzi-induced Chagas disease. A longitudinal study could provide the information necessary for monitoring sero-reconversion of seropositive subjects or long-term development of life-threatening complications of the infection.

### Example 4—Proteome Mapping the Chagas-Classifying Peptides

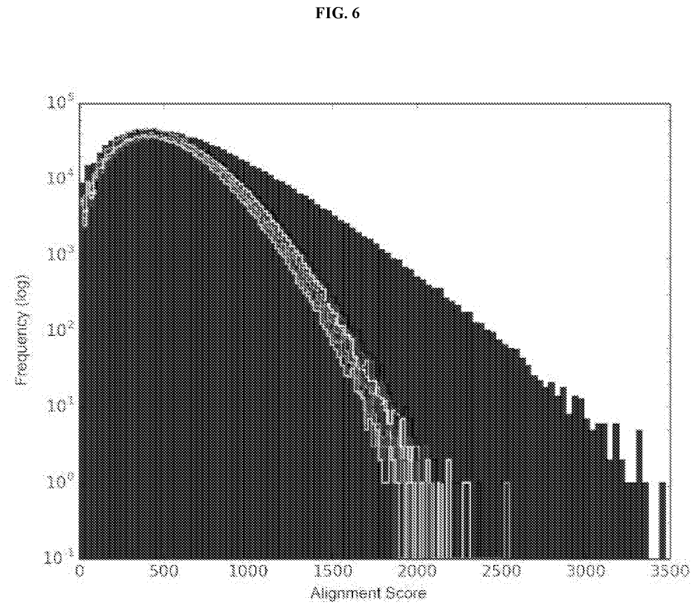

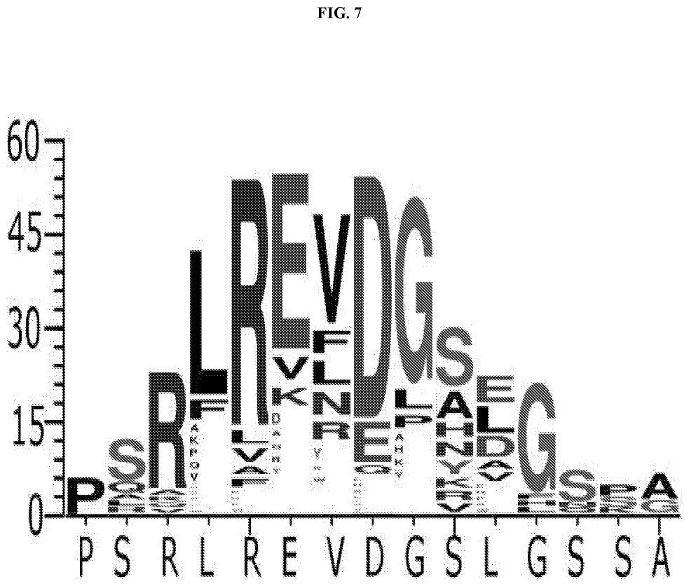

The 356 IST library peptides that significantly distinguished Chagas positive from negative donors plus the 14 that were correlated to S/CO values were aligned to the T. cruzi proteome with a modified BLAST algorithm and scoring system that used a sliding window of 20-mers (Example 1). This yielded a ranked list of candidate protein-target regions shown in Table 2. These classifying peptides display a high frequency of alignment scores that greatly exceed the maximum scores obtained by performing the same analysis with ten equally-sized (370) sets of peptides that were randomly selected from the library (FIG. 6). For example, the maximum score obtained with the randomly selected peptides ranged from less than 2000 to 2500; whereas the classifying peptides generated an alignment score of 3500. Thus, in this instance, the classifying peptides provided a protein score that was at least 28% greater than that of the highest scoring random peptide. Reliable results can also be achieved with a lesser degree of separation.

The top-scoring candidate mapped by the Chagas classifying peptides was the C terminus of the Mucin II family of surface glycoproteins. The IST peptide-aligned region includes a glycosylphosphatidylinositol (GPI) attachment site and corresponds to a highly immunogenic epitope in Chagas patients [Buscaglia C A et al., (2004) J Biol Chem 279: 15860-15869]. The amino acids's most frequently identified in the Mucin II-aligned IST peptides are summarized in FIG. 7 as a modified WebLogo [Crooks G E et al., (2004) Genome Res 14: 1188-1190]. The corresponding T. cruzi mucin sequence (UniProt ID=Q4DXM4) is displayed along the x axis. Amino acid substitutions at any one position are shown vertically and the proportional coverage within the mapped library peptides is depicted by the height of the one-letter code. Another member of the Mucin II protein family is identified as the sixth ranked target candidate, and it also maps to the C terminus (UniProt ID=Q4DN88). A member of another T. cruzi surface glycoprotein family, the dispersed gene family proteins (DGF-1) [Lander N et al., (2010) Infection and Immunity 78: 231-240], ranked eighth by the aligning algorithm (Q4DQ05), mapping to its C-terminal region and corresponding to the family's consensus sequence. The remaining top 10 scoring alignment regions mapped to proteins involved in calcium signal transduction (calmodulin), vesicle trafficking (vacuolar protein sorting-associated protein, Vps26) [Haft C R et al., (2000) Molecular Biology of the Cell 11: 4105-4116] and uncharacterized proteins. Together these 10 candidate proteome targets accounted for 220 of the aligned 370 IST classifying peptides. Leading candidate biomarkers can also be identified by up to all of the total number of discriminating peptides.

TABLE 2
Top ranking alignments of classifying library peptides
to T. cruzi proteome.

| | | | Amino acid |
|---|---|---|---|
| Rank | T. cruzi protein | UniProt ID | position |
| 1 | Mucin TcMUCII | Q4DXM4 | 170-190 |
| 2 | Uncharacterized protein | Q4DLV5 | 170-190 |
| 3 | Uncharacterized protein | K4EBQ9 | 950-970 |
| 4 | Calmodulin | Q4DQ24 | 110-130 |
| 5 | Uncharacterized protein | Q4D6B0 | 910-930 |
| 6 | Mucin TcMUCII | Q4DN88 | 340-360 |
| 7 | Uncharacterized protein | Q4DUA0 | 500-520 |
| 8 | Dispersed gene family protein 1 | Q4DQ05 | 3380-3400 |
| | (DGF-1) | | |
| 9 | Uncharacterized protein | Q4DCE7 | 220-240 |
| 10 | Vacuolar protein sorting-associated | K4DSC6 | 10-30 |
| | protein (Vps26) | | |

These data show that array peptides that mimic parasitic epitopes were bound differentially by peripheral blood antibodies in Chagas seropositive subjects. These discriminating peptides were mapped to several known immunogenic T. cruzi proteins, and to several previously unknown antigens.

### Example 5—IST Co-Classification of Chagas Positive Donors from Those Testing Positive for Other Blood Infectious Diseases: Chagas Disease, Hepatitis B, Hepatitis C, and West Nile Virus Disease

In addition to discriminating Chagas positive samples from Chagas negative samples, the immunosignature method was tested to determine whether Chagas disease could be discriminated from other infectious diseases, and whether the other infectious diseases could be discriminated from each other.

To determine whether Chagas positive samples could be discriminated by IST from other infectious disease samples, a subset of 88 samples from the full Chagas 2015 cohort was re-assayed, alongside 88 HBV, 88 WNV, and 71 HCV disease-positive plasma samples. The virus samples were assigned positivity by both indirect serologic and direct nucleic acid testing at Creative Testing Solutions. All study samples were reported as being positive for only one of the four diseases. The demographic data are presented in Table 3, showing mixed genders and ethnicities and a range of ages. A higher prevalence of Chagas positivity is seen among Hispanic donors, which is consistent with disease prevalence in Central and South America. This higher prevalence was also seen within the full Chagas cohort (Table 1). The distribution of ethnicities for donors testing positive for HBV, HCV and WNV were similar to the distributions found in the general U.S. population.

All IST assays for this study were performed on the same day and scanned immediately to acquire signal intensity measurements at each feature. The raw data was imported into R for analysis.

TABLE 3
Description of donors in the blood panel-positive disease study

| | all | Chagas | HBV | HCV | WNV |
|---|---|---|---|---|---|
| Group size (n) | 335 | 88 | 88 | 71 | 88 |
| Gender | | | | | |
| female | 62 | 27 | 7 | 7 | 21 |
| male | 102 | 30 | 11 | 21 | 40 |
| unknown | 171 | 31 | 70 | 43 | 27 |
| Ethnicity | | | | | |
| white | 70 | 5 | 2 | 16 | 47 |
| Hispanic | 54 | 38 | 1 | 5 | 10 |
| black | 5 | 0 | 4 | 1 | 0 |
| other | 18 | 4 | 11 | 2 | 1 |
| unknown | 188 | 41 | 70 | 47 | 30 |
| Age bin | | | | | |
| (16-20) | 11 | 3 | 3 | 1 | 4 |
| (20-30) | 30 | 7 | 6 | 7 | 10 |
| (30-40) | 26 | 14 | 2 | 2 | 8 |
| (40-50) | 36 | 11 | 3 | 6 | 16 |
| (50-60) | 35 | 12 | 1 | 10 | 12 |
| (60-70) | 18 | 6 | 3 | 2 | 7 |
| (70-87) | 8 | 4 | 0 | 0 | 4 |
| unknown | 171 | 31 | 70 | 43 | 27 |

Immunosignature assays were performed on all sample to identify the array peptides that were differentially bound by antibodies in samples from subjects infected with T. cruzi (Chagas disease), Hepatitis B, Hepatitis C, and West Nile. The array-based assay was performed as described in Example 1, on samples from subjects described in Table 3, and signal intensities of array-bound antibodies in each of the samples was acquired and analyzed as described.

Distinguishing an Infection from Another Infection

Differential antibody binding to array peptides identified peptides that discriminated Chagas (T. cruzii infection) from HBV, Chagas form HCV, Chagas from WNV, HBV from HCV, HCV from WNV, and WNV from HBV.

Comparisons of signal binding data obtained from samples from Chagas subjects to binding data from a group of subjects with HBV identified peptides that discriminated the Chagas samples from the group HBV were enriched by greater than 100% in one or more motifs listed in FIG. 14A relative to the incidence of the same motifs in the entire peptide library. Additionally, peptides that discriminated Chagas samples from HBV samples were found to be enriched by greater than 100% in one or more amino acids arginine, tyrosine, serine, alanine, valine, glutamine, and glycine (FIG. 14B). The method performance for this contrast was characterized by an 0.98 (0.98-0.99). At 90% sensitivity, the specificity of the assay was 96% (94-97%), the sensitivity of the assay at 90% specificity was 96% (94-97%), and the accuracy of the assay at sensitivity=specificity was 94% (93-96%).

Comparisons of signal binding data obtained from samples from Chagas subjects to binding data from a group of subjects with HCV identified peptides that discriminated the Chagas samples from the group HCV were enriched by greater than 100% in one or more motifs listed in FIG. 15A relative to the incidence of the same motifs in the entire peptide library. Additionally, peptides that discriminated Chagas samples from HCV samples were found to be enriched by greater than 100% in one or more amino acids arginine, tyrosine, serine, valine, and glycine (FIG. 15B). The method performance for this contrast was characterized by an 0.99 (0.98-0.99). At 90% sensitivity, the specificity of the assay was 94% (92-98%), the sensitivity of the assay at 90% specificity was 98% (95-99%), and the accuracy of the assay at sensitivity=specificity was 93% (92-95%).

Comparisons of signal binding data obtained from samples from Chagas subjects to binding data from a group of subjects with WNV identified peptides that discriminated the Chagas samples from the group WVN were enriched by greater than 100% in one or more motifs listed in FIG. 16A relative to the incidence of the same motifs in the entire peptide library. Additionally, peptides that discriminated Chagas samples from WVN samples were found to be enriched by greater than 100% in one or more amino acids lysine, tryptophan, aspartic acid, histidine, arginine, glutamic acid, and glycine (FIG. 16B). The method performance for this contrast was characterized by an 0.95 (0.94-0.97). At 90% sensitivity, the specificity of the assay was 87% (76-94%), the sensitivity of the assay at 90% specificity was 89% (85-92%), and the accuracy of the assay at sensitivity=specificity was 90% (86-91%).

Comparisons of signal binding data obtained from samples from HBV subjects to binding data from a group of subjects with HCV identified peptides that discriminated the HBV samples from the group HCV were enriched by greater than 100% in one or more motifs listed in FIG. 17A relative to the incidence of the same motifs in the entire peptide library. Additionally, peptides that discriminated HBV samples from HCV samples were found to be enriched by greater than 100% in one or more amino acids phenylalanine, tryptophan, valine, leucine, alanine, and histidine (FIG. 17B). The method performance for this contrast was characterized by an 0.91 (0.88-0.94). At 90% sensitivity, the specificity of the assay was 79% (69-86%), the sensitivity of the assay at 90% specificity was 71% (53-83%), and the accuracy of the assay at sensitivity=specificity was 84% (78-87%).

Comparisons of signal binding data obtained from samples from HBV subjects to binding data from a group of subjects with WNV identified peptides that discriminated the HBV samples from the group WNV were enriched by greater than 100% in one or more motifs listed in FIG. 18A relative to the incidence of the same motifs in the entire peptide library. Additionally, peptides that discriminated HBV samples from WNV samples were found to be enriched by greater than 100% in one or more amino acids tryptophan, lysine, phenylalanine, histidine, and valine (FIG. 18B). The method performance for this contrast was characterized by an 0.97 (0.96-0.98). At 90% sensitivity, the specificity of the assay was 96% (90-99%), the sensitivity of the assay at 90% specificity was 94% (90-97%), and the accuracy of the assay at sensitivity=specificity was 93% (90-96%).

Comparisons of signal binding data obtained from samples from HCV subjects to binding data from a group of subjects with WNV identified peptides that discriminated the HCV samples from the group WNV were enriched by greater than 100% in one or more motifs listed in FIG. 19A relative to the incidence of the same motifs in the entire peptide library. Additionally, peptides that discriminated HCV samples from WNV samples were found to be enriched by greater than 100% in one or more amino acids lysine, tryptophan, arginine, tyrosine, and proline (FIG. 19B). The method performance for this contrast was characterized by an 0.97 (0.95-0.98). At 90% sensitivity, the specificity of the assay was 92% (84-97%), the sensitivity of the assay at 90% specificity was 93% (86-97%), and the accuracy of the assay at sensitivity=specificity was 92% (87-94%).

These data show that comparisons of individual infections can be made using the immunosignature assay described herein to differentially diagnose many different infectious conditions.

Distinguishing One Infection from a Group Comprising Two or More Different Types of Infection

Binary classifiers were developed for differentiating each of the available infectious diseases from the combination of the others (Table 4). Performance metrics of each disease contrast and their corresponding 95% CI's were determined by four-fold cross-validation analysis. The models generated similar strong AUC's, which ranged from 0.94 to 0.97, and corresponded to accuracies of 87%-92%. Nominally, the contrast of Chagas disease versus the combined class of the remaining three diseases (other) was best performing; however, the parenthetically shown CI's overlapped. Nominally, the hepatitis contrasts were the weakest models. The number of optimal SVM input peptides varied widely from 50 to 16,000 peptides.

Differential antibody binding to array peptides identified peptide that discriminated Chagas samples from a group of mixed samples from subjects having HBV, HCV, and WNV (other). The most discriminating peptides were found to be enriched by greater than 100% in one or more motifs listed in FIG. 10A relative to the incidence of the same motifs in the entire peptide library. Additionally, peptides that discriminated Chagas samples from the group of HBV, HCV, and WNV samples were found to be enriched by greater than 100% in one or more amino acids arginine, aspartic acid, and lysine (FIG. 10B).

A binary classifier was developed based on the binding signal information of discriminating peptides, and was shown to clearly differentiate samples from Chagas disease subjects from samples from the other infectious diseases, HBV, HCV, and WNV, with an assay performance characterized by an AUC=0.97. At a 90% confidence level, the specificity of the assay was 94%, the sensitivity of the assay was 92%, and the accuracy of the assay was 92% (Table 4).

Comparisons of signal binding data obtained from samples from HBV subjects to binding data from a group of subjects with Chagas disease, HCV, and WNV identified peptides that discriminated the HBV samples from the group of Chagas disease, HCV, and WNV, which were enriched by greater than 100% in one or more motifs listed in FIG. 11A relative to the incidence of the same motifs in the entire peptide library. Additionally, peptides that discriminated HBV samples from the group of HBV, HCV, and WNV samples were found to be enriched by greater than 100% in one or more amino acids tryptophan, phenylalanine, lysine, valine, leucine, alanine, and histidine (FIG. 11B). The method performance for this contrast was characterized by an AUC 94%. At a 90% confidence level, the specificity of the assay was 85%, the sensitivity of the assay was 85%, and the accuracy of the assay was 87% (Table 4).

In a third set of contrasts, comparisons of signal binding data obtained from samples from HCV subjects to binding data from a group of subjects with Chagas disease, HBV, and WNV identified peptides that discriminated the HCV samples from the group of Chagas disease, HBV, and WNV, which were enriched by greater than 100% in one or more motifs listed in FIG. 12A relative to the incidence of the same motifs in the entire peptide library. Additionally, peptides that discriminated HCV samples from the group of HBV, HCV, and WNV samples were found to be enriched by greater than 100% in one or more amino acids arginine, tyrosine, aspartic acid, and glycine (FIG. 12B). The method performance for this contrast was characterized by an AUC=96%. At a 90% confidence level, the specificity of the assay was 91%, the sensitivity of the assay was 90%, and the accuracy of the assay was 90% (Table 4).

In a fourth set of contrasts, comparisons of signal binding data obtained from samples from WNV subjects to binding data from a group of subjects with Chagas disease, HBV, and HCV identified peptides that discriminated the WNV samples from the group of Chagas disease, HBV, and HCV, which were enriched by greater than 100% in one or more motifs listed in FIG. 13A relative to the incidence of the same motifs in the entire peptide library. Additionally, peptides that discriminated WNV samples from the group of HBV, HCV, and Chagas samples were found to be enriched by greater than 100% in one or more amino acids lysine, tryptophan histidine, and proline (FIG. 13B). The method performance for this contrast was characterized by an AUC=0.96. At a 90% confidence level, the specificity of the assay was 88%, the sensitivity of the assay was 87%, and the accuracy of the assay was 89% (Table 4).

TABLE 4
Binary classification of each of four disease classes versus a
combined class of the remaining three.

| | | | | accuracy |
|---|---|---|---|---|
| | | sensitivity | specificity | @ sens^{b} = |
| | AUC | @ 90% spec^{a} | @ 90% sens^{b} | spec^{a} |
| Chagas vs. | 0.97 | 92% | 94% | 92% |
| Other | (0.96-0.98) | (90%-94%) | (90%-96%) | (90%-92%) |
| HBV vs. | 0.94 | 85% | 85% | 87% |
| Other | (0.93-0.95) | (78%-90%) | (78%-90%) | (85%-90%) |
| HCV vs. | 0.96 | 90% | 91% | 90% |
| Other | (0.94-0.97) | (86%-94%) | (82%-96%) | (88%-93%) |
| WNV vs. | 0.96 | 87% | 88% | 89% |
| Other | (0.95-0.97) | (78%-94%) | (84%-92%) | (86%-91%) |

^{a}spec, specificity;
^{b}sens, sensitivity

These data show that binary classification of a plurality of different infections based on identified discriminating peptides can distinguish subjects that are seropositive for Chagas from subjects that are seronegative for Chagas, and from subjects that are asymptomatic for WNV, HPV, and HCV. As shown, in every instance, the method performance is greater than 0.94.

### Example 6—Simultaneous Classification of Four Different Infections

A multiclassifier model was developed to classify all four infectious disease states simultaneously, with one set of selected peptides, and one algorithm. This multiclass model had similar performance to the binary classifiers shown in Table 4. Namely, the four-fold cross validation analysis yielded multiclass AUC's of 0.98 for Chagas, 0.96 for HBV, 0.95 for HCV, and 0.97 for WNV. Table 5 presents the performance metrics of the assignments of each sample to a class based on its highest predicted probability. In this confusion matrix, each binary contrast is presented. The estimated overall multiclass classification accuracy achieved 87%.

The classifiers for the group contrasts described in the preceding paragraphs and Table 5 were combined to obtain a multiclassifier to determine whether the four infections: Chagas, HBV, HCV, and WNV could be simultaneously discriminated from each other.

Peptides discriminating Chagas, HBV, HCV, and WNV samples from each other in the multiclassifier analysis were enriched by greater than 100% in one or more motifs listed in FIG. 20A relative to the incidence of the same motifs in the entire peptide library. Additionally, the peptides that discriminated Chagas, HBV, HCV, and WNV samples from each other in the multiclassifier analysis were enriched by greater than 100% in one or more amino acids arginine, tyrosine, lysine, tryptophan, valine, and alanine (FIG. 20B).

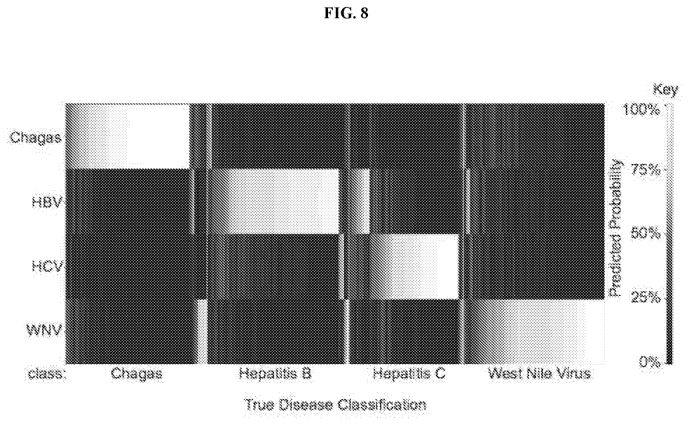

The heat map shown in FIG. 8 visualizes the mean predicted probability of class membership of out of the bag cross validation model predictions (shown in Table 5) for each of the 335 test cohort samples, encompassing all four diseases. This figure demonstrates that the highest predicted probabilities correctly assigned samples to the infectious disease class. Signal intensities of the classifying peptides are visibly more different in the Chagas samples relative to all three of the virus sample. Most, but not all, are higher in Chagas with notable exceptions for a few lower peptide signals relative to HBV and WNV. By contrast, the differences in signal intensities for the same peptides assayed against HBV and HCV samples are less extreme.

Each sample has a predicted class membership for each outcome ranging from 0 (black) to 100% (white). Each sample was assigned to a disease class based on the highest predicted probability presented in FIG. 8 and show in the confusion matrix given in Table 5. The classifications were assigned based on the predicted probabilities shown in FIG. 8 with each sample being assigned to the class with the highest probability. The assay performance for the four contrast ranged from 0.95 to 0.98. The overall accuracy was 87%.

TABLE 5
Confusion matrix and Performance Estimates for multiclass predictions

| | ImmunoSignature Classification | | | | Performance | | |
|---|---|---|---|---|---|---|---|
| Confirmed | | HBV | HCV | WNV | Summary | | |
| Diagnosis | Chagas pos | pos | pos | pos | Sens | Spec | AUC |
| Chagas | 77 | 3 | 1 | 2 | 93% | 96% | 0.98 |
| HBV | 3 | 79 | 12 | 2 | 82% | 96% | 0.96 |
| HCV | 0 | 3 | 55 | 2 | 92% | 94% | 0.95 |
| WNV | 8 | 3 | 3 | 82 | 85% | 97% | 0.97 |

Overall accuracy = 87%

These data show that the immunosignature assay can simultaneously distinguish one infection from two or more other infections with a high degree of accuracy. In all instances, the method performance as defined by the AUC was greater than 0.95.

### Example 7—Immunosignature Assay Differentiates Subjects that are Seropositive for T. cruzi from Subjects that are Seronegative for T. cruzi Using an Expanded Peptide Array

To identify additional array peptides that could differentiate samples that are seropositive for T. cruzi from samples that are seronegative, a 3.3M feature array of 3.2M unique peptides (V16 array) was used for the binding study. The V16 array comprises a library of peptides synthesized from 18 of the 20 naturally occurring amino acids by excluding cysteine (C) and methionine (M). Peptides are median length 8, and range from 5 to 16 amino acids in length. The libraries on the V16 array include: (A) a low-bias library, which is a high sequence-diversity library of unique peptides designed to cover sequence space evenly based on the 18 amino acids that includes pentamers, hexamers, septamers, and octamers, and their monomer, dimer, trimer, and tetramer subsequences; (B) a V13 library, comprised of 88,927 full-length peptides from the array library described in Example 2, and between two and four fragments of another 37,098 peptides from the array library described in in Example 2; and (C) an IEDB library of 274,417 unique epitope sequence peptides targeting epitopes in the International Epitope Data Base (http://www.iedb.org/). The IEDB library comprises 2,951 unique peptides mapped to epitopes of proteins of the T. cruzi organism.

Plasma samples were obtained from Creative Testing Solutions (CTS; USA) (at www.mycts.org). Binding assays were performed using 49 samples from asymptomatic donors known to be seropositive for Chagas having an S/CO score of at least 1.245, and 41 samples from seronegative donors. Six additional replicates of one of the seronegative donors were also included in the binding assays. The binding assays were performed, and sample antibody-to-peptide binding was detected as quantitative signal measurements that were obtained by determining a relative fluorescence value for each addressable peptide feature, as described in Example 1.

To construct a classifier, features were ranked for their ability to discriminate Chagas seropositive from the seronegative samples based on the p value associated with a Welch's t-test comparing Chagas positive to Chagas negative donors. The number of input peptides selected was varied between 25 and 16,000 features in steps and each set of the selected features was input to a support vector machine (Cortes C, and Vapnik V. Machine Learning. 1995; 20(3):273-97) with a linear kernel and cost parameter of 0.01 to train a classifier. A five-fold cross validation repeated 100 times was used to quantify model performance, estimated as the error under the receiver-operating characteristic curve (AUC), and incorporated both feature selection and classifier development to avoid bias.

All analyses were performed using R version 3.3.3 (Team RC. R: A language and environment for statistical computing. R Foundation for Statistical Computing Vienna 2017. Available from: https://www.R-project.org/.)

The Volcano plot visualizing a set of library peptides displaying antibody-binding signals that are significantly different between Chagas seropositive and Chagas seronegative subjects is shown in FIG. 22. The volcano plot is used to assess this discrimination as the joint distribution of t-test p-values versus log differences in signal intensity means (log of ratios). The density of the peptides at each plotted position is indicated by the heat scale. The 2,707 peptides above the red dashed line discriminate between positive and negative disease by immunosignature technology (IST) with 95% confidence after applying a Bonferroni adjustment for multiplicity. The blue colored circles indicate the differential binding of seropositive and seronegative samples to peptides in the IEDB library targeting epitopes of Chagas disease. The 67 discriminating peptides shown by blue circles above the blue line discriminate between positive and negative disease with 95% confidence after applying a Bonferroni adjustment for multiplicity. The green circles represent the 493 peptides bound by sample antibodies to peptides of the V13 library. The 52 peptides shown by green circles above the green line discriminate between positive and negative disease with 95% confidence after applying a Bonferroni adjustment for multiplicity. Three Bonferroni cut-off values were used, adjusted for the sizes of the 3 subsets of peptides on the V16, V13, and IEDB libraries.

The discriminating peptides from the V16 array analysis are listed in Table 6 below. The peptides are ordered by increasing p-values for a t-test of the difference in mean log-transformed intensities of subjects who were Chagas seropositive and mean log-transformed intensities of subjects who were Chagas seronegative. The hash-tag symbol (#) identifies discriminating peptides from the IEDB library that were designed to map to reported Chagas epitope sequences, and the asterisk symbol (*) identifies peptides from the V13 library of V16 that are listed in FIGS. 21A-N. Each unique peptide's sequence is followed by the ratio of the mean seropositive over mean seronegative intensity for that peptide.

TABLE 6
Peptide Sequences Discriminating Between Chagas Seropositve Samples from Chagas
Seronegative Samples

TIRKIDA 35.61, YIRKIDPS 32.05, IYRKIDG 28.78, LRKIDSL 26.27,
LIRKIEA 30.75, ILHRKIDEV 28.7, AIRQID 18.17, LRKVD 30.82,
IVRKIDYQ 23.41, IIRKVDVD 20.41, LRAVDPVG 16.88, ITVRKID 23.25,
IRKIYDNV 20.03, PGKDTKPA 9.97, IRDKIDTF 14.73, LRKIDSNS 25.71,
DKLREIDK 18.01, IRKIETVD 15.84, LREIDEGF 17.63, DKIRQIDG 15.16,
LYRKIDS 27.61, DLRTKIDS 19.37, IRAIDPYT 13.58, PGKEVKK# 10.61,
EIARKIDY 13.98, VIRKVEGDI 15.85, IPGKENKY 16.96, LRKLD 22.03,
PGKPEIFKS 9.85, IRKIGDTSVS 22.22, IARLIDPG 12.62, PGKAQLKE 12.45,
ELIRKIE 18.3, LREVDADGDL 19.31, DIRKLDY 14.82, PGKEQKVI 10.84,
QAAAGDKP# 13.98, IQRRKIDV 18.83, PGKGTKENL 10.58, DLREIDPA 15.25,
IRRRIDT 19.69, IRKPIDYTI 13.45, DPGKQIK 14.57, IRKPIDYTV 10.43,
DQLRKIID 14.39, LLRKVDSDL 17.06, HRIRKIDI 19.53, RDLRRIDP 13.91,
IRKIEAY 21.06, LQRKIEA 23.05, PGIRKELK 10.27, LVREIDQE 17.81,
PIGKDLKI 11.53, IRRRIDINP 15.95, HRDLRQID 14.5, IRAIEAPD 16.36,
DLRSIDSP 17.52, PGKELTRQ 9.75, WITRKLID 16.58, AFRIRLID 11.09,
ALRLIDSG 13.76, HLRDKIDG 18.56, YNPGREIK 12.02, VREIDK 16.58,
LREIDGSLS# 10.13, LYRRIDG 16.29, IREKIDGV 17.5, RDLRRVDG 10.28,
TVRKIDA 16.38, RIQRKIED 18.89, AVLRAIDG 13.18, APGIRKELK 8.91,
RIDRKIE 17.35, PGPPKDLKVS 8.31, IRKIGEAE 18.52, PGKEFLKI 8.78,
WVRAIDV 9.75, KQIRLIE 13, PNGKLETK 10.49, IYRRIDG 15.94,
NLGRKIDE 24.93, PGWGKEQK 10.83, PGEVKERK 9.07, DTIRLIDA 13.03,
LRLVDGGG 13.67, VRAIDLP 9.34, LKRAKIDE 22.06, ALDRKIDP 18.62,
IRKIDQRVLE 12.86, LQRKLDE 20.47, ITRKIKDSDA 20.72, LQRLIDS 14.87,
DLARQIDT 15.48, QLGREID 18.25, IRWTKIDE 13.27, IRQQIDG 20.75,
YKELRKID 21.31, FLPRKIDG 15.87, YIRLIDGV 9.02, GFQREID 12.29,
LREVDQVDG* 15.55, RLREIDG# 11.34, LRRELDAS 14.75, YIREIDSN 15.68,
LTFREIDS 16.53, LRRKLEDG 14.78, RLRKVDDA 18.74, IYRAIDG 15.8,
IRGQRQID 15.56, ALVYRRID 10.78, GIRLIDV 10.92, IIRKFIE 15.71,
LRLVDADDP 12.53, IVLRRKVDE 18.69, IRQIDDI 13.08, PGKSLKEN 10.28,
YFREIDTKD 13.96, PGSELKIK 8.77, IQERKIDD 19.58, IRKLDSAL 14.01,
HLRDIDGN 15.34, LRRIDEAT 14.67, LRSEIDNVK 16.06, LRQVDDTG 19.38,
FDQRRQVD 11.3, RLREVDGS 13.64, PGLKWDLK 9.36, NLNREIDT 19.24,
VRAIDE 12.08, DRLRQIEA 14.72, LRKLEAAE 17.06, PGTETKSG 5.62,
AIRYRIDT 11.94, KLREIEEV 14.76, FVRAIDV 9.59, LREVKDEV 17.24,
VIKRKIEPLEV 14.87, HNIRDID 17.25, DFRAIDGI 7.63, QIRLIENGS 14.43,
DIVRLIDG 10.9, IRKFIDT 16.43, LLTREVDDT# 15.4, LRAKIDLSS 17.9,
IREVDQAG 19.1, LIRLIEDG 11.31, IAIRRRIE 14.09, PGKLLKE 8.33,
HRVIRQID 11.93, IGKETIKSS 14.82, QIRLIEK 16.78, GRIREIE 11.6,
HYLRAIDG 14.93, DLRQIDPA 14.08, PGKDGKP 8.61, LRALDQTPGSS 12.24,
TLRLIEPV 10.67, HHLRRVD 13.51, YSREIDTE 13.95, YLRGQIDV 14.45,
DQRAIDPA 12.7, LRLVDADD 14.27, HIRQIDWP 8.84, AILRTKID 16.16,
ILRELDVE 13.07, HTYIRRID 10.83, HDSVNIT 4.02, IRLIEAVD 9.36,
VLKREIDK 18.7, PSGRETKG 9.02, ILRALDST 16.07, LREVEEPD 15.41,
IIRKLDF 12.87, PGSFKEAK 9.8, AQIHRKIE 16.64, HFREIDVE 13.34,
STLRKIE 17.38, SPGWKERK 10.77, PGEKQTKP 8.41, LQRRIDY 12.49,
QVQLRKIE 16.47, LDRKIET 14.93, LREVDPWN 6.96, LRDEIDQF 11.82,
GYIRKIEL 14.71, INRRIDVI 11.31, APGYKHEIK 10.42, LTVREIDH 15.13,
IESRKIDQ 14.04, DITIRKLD 11.77, SIIRLIE 11.43, HRPIRKIE 14.3,
QLRQEIDQ 20.27, KLVRKVDEP 16.41, SLRKLEPE 14.8, DDLRAHID 11.88,
IRAVDGTIAG 12.24, LREIEYAE 13.91, NIRDIDV 14.95, PGKWDAQK 7.08,
LRELDDFT 11.35, LRHVD 16.31, PGPSKDIKAS 8.13, RLREIDGS# 11.81,
LERKIDWN 14.59, GREIDNFV 10.08, DLRAIDEE 14.05, IPGKQAKG 8.66,
YLRQVEAP 16.1, LRRDIDDLE 13.57, TDLYRKIE 13.41, LYRQIDQP 13.46,
IRHEIDAD 15.6, ALHRKIEI 17.67, LKREKIDGV 14.19, IRLVEDGK 12.17,
FWRKIDTE 15.28, LRKLDHISES 15.12, IIRLLDS 11.86, FTRKIDVE 13.97,
LDREVDPVD 14.06, YLQRHRID 14.1, LREITDK 12.74, IRRLVDT 13.21,
DKPIREID 7.5, LRELIDQ 15, IRRIETEG 11.55, LNRIID 9.57, IARLVDDP 8.97,
IWRKIVDI 9.84, DLRGESIDVDES 13.75, DIRQNIDI 12.47, LHRRQIEP 17.07,
GIRDIEAI 9.51, LTREKIDGV 14.42, RLDRKIE 13.99, LRQIDGQT 15.95,
HFPVRKID 11.33, DFKRLQID 9.22, PVLRKIEEV 8.35, LRLLRRVD 12.14,
IQRQRNID 15, IHIRSIDV 13.4, NALRKIDT 19.07, KLLRQVD 15.72,
LRKHIDES 20.64, TQLRRHID 14.8, GIRLIDI 9.12, FLRKIYDA 15.55,
YFLRKNID 16.06, YTLREVDTV 11.03, VQRKVDAE 13.08, LRLLID 10.72,
IRIRLIDH 15.85, IRYIDTDD 13.95, IIRLLEGANP 10.24, LKREEIDG 12.63,
GRLIDFP 7.98, SHIREIDP 13.08, IIRLLESS 10.41, INRIIDGE 8.48,
IRPKIDSH 11.11, IRKINWDG 10.83, GVRLRQVD 11.77, LARQVDG 14.79,
NIREIEI 14.96, LRLIDGQTS 12.17, HIVQREID 13.23, LIHRLIE 12.31,
IRKVEWPDL 7.53, APIAREID 7.33, GYREIDYI 8.36, IPGKAENK 10.65,
GPIARRIDG 7.64, IRRFIDT 13.05, PTGKEPIK 6.39, RLREVDKY 11.77,
VREIDIAS 11.58, ILRQQIDP 18.81, KLREIEDQ 14.32, DNHIRLIE 13.73,
NLLHREVD 13.04, PEGKHQVK 8.63, INRSIDDE 14.94, LLLTREVD# 12.83,
GLRKVID 14.52, LAREVDLKDY 10.55, LRKIFDGY 13.59, LPWLREID 8.14,
IQGRQIDY 10.72, LIRELDGV 8.54, TALRKRID 15.98, LGRSIDDIG 10.36,
LESREIDA 14.36, IFGFREID 10.55, LARQVDGD 13.85, DYLIRRLD 8.89,
DLLRSIDSG 13.67, IRTNIDES 16.77, YIKRAIDS 14.5, LRKVETSL 14.06,
LGIRAIDP 13.05, RIRKIEWE 6.53, LRKLDLIE 9.91, PGKQQKP 7.16,
DIRKLLDI 9.27, AFILRRIE 12.44, NIREIEE 15.77, QLKRQIDD 15.5,
DLRLVENA 10.44, AGLHREIE 11.7, PGFREVYK 6.7, APGKGLEQKR 8.64,
LSRELDF 9.4, IARDQIDS 13.33, YIFRQQID 14.94, GFLRHKID 17.3,
LLRKIYE 12.01, YGLRAIEP 13.12, LRRFIDGP 12.6, DIRKLLDS 8.55,
AREIDESL 11.01, RIRKVGDIE 8.42, LIRLVESS 12.08, IRHKIEEK 15.84,
RIRRHIDA 14.46, HFAKREID 13.79, LSQKRQID 15.48, LREVEPWKE* 7.86,
LDREVDVW 11.18, DLRKRIEAF 11.3, WVQRKVDDG 11.72, KRIFRRID 12.37,
HIIRKLEE 11.84, YDFRKVD 8.69, LRDQIDPIL 10.14, DSLRREIE 10.29,
HIRFIDDV 9.25, LWWYRDID 8.86, LRELDDQE 12.46, IRRIDTEW 5.66,
LRLLDDTK 11.64, WIRHNIDG 13.63, PGKGLEVK 6.1, IRLIDKL 14.67,
QLEIRKID 14.13, VLRREIES 12.3, VPGKQTKS 7.45, YRDTYVVH 2.3,
GIRAIEGN 12.65, ITDRKIEY 10.21, YIRNIDGE 10.22, LRSIDLVSSV 10.46,
LRLLDPTS 11.67, RILRQIEGL 8.33, IREKIEDAK 14.77, LLRKINSEP 11.39,
WQSLRRID 10.8, DIRDIIDS 8.03, ATREIDKP 10.05, ELRSIDPP 13.11,
SLRLIENG 11.92, LLRETDGP 11.82, WQIRAIDN 13.92, KLKRQEIDG 14.26,
LRVIDSAA 11.7, SLRLVDA 11.92, DNDPKNWT 2.44, LRALDELP 8.87,
LRRREIEP 9.37, LHRQVDGT 18.2, QRRIRYID 9.28, LRTAIDQ 13.51,
LRANIDNI 13.66, VIRQRLVD 8.96, PDTGWKHERK 6.03, ILRSEIDS 12.97,
LNRKIEVL 13.86, GIDSKH 2.34, ISRDIDTA 12.66, SPVGKEHK 10.11,
SLREIKDF 10.91, LRDVDEAAV 12.8, LRGLDGPAA 10.42, DYVRAIDA 6.76,
AIWREIEV 7.91, SLIREVDK 12.11, AIKRKIDN 12.3, YFGHREID 13.19,
DGRLIDTG 9.59, IREIELK 11.21, IRGLIEEL 9.46, DTRRIDGY 5.21,
LRRSVDTSS 13.4, IRTKIEQS 13.29, IDRQIENF 7.71, NLNRKIEDG 14.99,
LRKVGDSV 11.69, YPGKQSKP 8.26, LRAEIDLG 7.2, ALRNLIDG 10.01,
ALIRLIEDG 9.7, LRQGLIDTS 14.3, LRREVEK 12.29, IRQILDEAG 12.44,
LQRLLD 9.41, IIRLIESARP 11.25, DGRLIDS 9.44, LRNITDEP 12.81,
LHREVEGV 12.25, LRAVEPALL 10.85, VRKIDINQ 11.74, IRDLDSGTV 9.87,
LIRLINEES 11.11, ATYLRAID 8.18, LHRELDYT 9.5, LIWHRSID 13.45,
DRSLRIID 7.58, FLQRRLIE 10.92, LRALEEP 11.1, ADLRRLD 7.56,
IWRDIDF 7.59, DILRNIDG 11.55, FLRKIHEE 12.11, LRLIDDFT 9.25,
YDQRWRID 5.5, NLRRIDSL 11.45, IRLIEKQ 14.64, ILRKIETFL 8.88,
IPQKRKID 9.87, LRSIEEKA 14.13, GLHDSTS 2.65, LRTIDDFG 7.9,
AQSREID 11.01, IRDRQHLH 1.87, YTPGRENK 10.17, PGKEDKRYGP 5.1,
KLSRLIE 12.93, LRAKVDELLE 11.6, VQKREIDY 9.6, IIRLLDG 9.68,
LLRKHIDI 13.21, DSWLRKVE 8.45, LDRYQRID 7.8, DIRSIDGQ 12.61,
QRRKIDNE 13.73, ELRREVDT 11.04, LARIIDS 10.05, LRAVDSEYP 7.82,
LRRQVEVLT 9.77, ILYRQIDN 14.46, FREIDQKW 9.29, PILRLIDP 7.26,
NQDLRLID 12.23, LLRALDN 10.29, GLRLVDPQ 10.22, DVWIHHVQ 2.09,
YQLRQIDV 11.68, INRSQIDV 16.37, IYRKQVDY 10.78, LLRALDNGLG 8.99,
LAREVDLKDYE 10.5, NFRQRLID 10.18, LARRLD 10.06, IARAIDWG 8.27,
LRELIEES 11.85, PGREAQKR 7.9, YLRNIDGE 10.39, LRAIDPDEG 13.41,
ILRDVIDGG 9.34, WLQQRAVD 12.8, KIRDIDAATE 10.03, ARREIDAF 5.42,
DFYFRQID 9.49, IGRQKIDG 13.73, LRKPLDFETK 6.07, DLRQTIDF 8.78,
AQRKIDSF 13.41, GARRIDF 7.66, LKRQVDEAEE 16.23, NLARKIESEV 13.34,
HVTLREVD 10.33, YRLQRKIE 14.65, QLIRKILD 10.76, DLRDQVDG 10.29,
LYRKDIDY 8.45, QRLLD 11.43, DLREEIDY 11.6, GVIINIGH 1.88,
IARTIDES 11.03, LRLVDGQAS 10.42, DRDHSVLH 2.44, DQSLRKLD 9.57,
VYRIRHID 8.73, LRIIDSK 11.85, LNRLIDK 15.3, IRLKIDLY 8.42,
RPGKGQKEG 7.47, GIRQIDFV 7.83, LDRRLDV 6.46, LRDLIDKQT 12.52,
LFKRLID 11.65, PGSRDIKS 7.46, LGRRIDNL 11.18, LRTLIDQ 10.86,
RLQRKIE 13.41, GIRRLDV 8.41, QKIRRQIE 12.69, ARLVD 7.53,
IWRDIDFA 7.54, RGRIRRVD 10.97, FQPQRKID 11.51, LKRELIDI 9.64,
NVVHHHI 2.42, LDIRALDSP 9.73, DYDRGRYI 2.09, LKRKLEGDASDF 10.52,
WIREIHDN 12.65, IRSIDVTI 8.47, NLRRKVED 13.23, LHSREVDG 10.9,
IRAVETPE 10.7, DKLTTREIE 6.8, SDLRKLD 12.44, DDKGSKVQ 2.82,
IKTRKIDA 10.72, IQRLIEQEE 11.95, LTRRELDI 7.32, LRTAIDQVS 11.67,
TISRSIDY 8.14, WQHRKIDL 14.67, YLRANIDG 12.43, GIRLIDIA 6.78,
GLRSYIDNI 8.12, GDIHESSL 2.58, LGRQIDNG 15.01, RALRLVDGG 12.26,
YIRKINELLP 9.16, WFRRGQID 11.38, LRHQIEAS 17.32, WELERKID 11.45,
GYIREIEATG 7.08, IRALIDYD 8.71, YLLRAVDV 9.84, LRSVDWIP 8.6,
LIRKFDAG 9.02, YRDRQIDL 6.61, DSDYSIHH 2.02, WLYREIGDS 8.41,
KSLRRIDP 13.77, WVGKDIKV 10.35, QLREKVDFEG 9.9, IHLRSIDE 13.34,
YIRTNIDY 8.46, IYRQRIDF 8.79, QAAAGDKPS# 7.93, FRAIDGNG 11.97,
TLRKIVDI 10.95, LHFRKIEE 14.43, GWADHLYQ 1.93, NRNRIRLIEG 8.8,
DALRTLIDQ 8.72, NLVRLIDN 10.79, RIGVRSID 9.63, IRLLDGIV 9.13,
LRRSVDTSL 10.44, LRKIGEYQ 10.36, LRKLDIKVE 8.85, NLVRKIEVG 11.61,
KRLRREIE 9.58, FKIRRIDY 11.62, ILRNIDSH 13.84, IHYRTIDS 11.31,
IRLNIEE 12.2, IIRLLESA 8.82, IFRRTIDS 13.19, IQVGKEVKTGS 9.96,
HIRTIDVI 8.39, DLRRKQIE 11.9, LRATDPDVG 9.97, VWIRFKIDAS 10.45,
IIRLLESATP 8.55, IRQIDKSS 14.57, NHLRRKIE 15.8, WQHIREIE 10.85,
GRKIDALP 12.06, ILRSIEGEL 8.66, DSYRAIDT 5.77, LDRSIEVP 12.6,
HAREIDDE 11.22, LRELDLQV 9.57, IRALIEEVA 8.13, FREIIDQ 8.43,
NLNRKVDDG 12.67, GRDIDYGG 5.68, GLRAIEI 9.73, QIRDVDFA 8.11,
IPGKLVKG 6.57, LRELDLPSQ 9.07, WAIRAIET 8.84, KLNRLIE 10.59,
YLRRIIDQ 8.87, LRRVIDTS 8.54, PGLKGLKGLP 6.63, PGKSELR 5.99,
IIRLLEDAKP 8.06, LRVKIHDA 9.95, DLLRLIDYN 9.05, IRLLDFPT 8.62,
IFKIRELD 9.11, LRGAVDIDDNG 11.34, LLVRQIEG 9.27, FLRVIDGG 9.58,
DDFHTGKI 2.9, LRWLIDSQ 10.34, LSRRIDAL 9.08, LTKTRAID 12.4,
TLRLLD 8.34, DIRLNIDF 7.14, RYLREIET 9.08, FLRKIYEE 9.34,
LKRPEIDW 6.73, IREVIDHL 9.44, LDTRDIDL 11.47, YLERNKIDVNE 12.06,
LARQVDGDN 12.31, AIGNRSID 9.28, VIRAIEE 9.71, LRQLDLDV 8.98,
IIRTIDQL 8.15, DGIRQIEV 8.23, QIRTQIE 12.07, LKDRLIDP 10.5,
YIFRIIDG 9.19, WIRAIDDN 11.58, DLRVIDFNST 5.8, GLKRDIDD 11.27,
AGPLRLLD 5.88, YKREIDEE 10.5, PGKDWIAK 5.24, VIGRQIEG 8.65,
LRLINSGD 10.41, IRARVNID 8.92, KSHHVHHI 2.24, LRNLDLAP 9.55,
IRAVEET 11.37, QRAIDGVT 11.5, IRKIDDNR 9.35, IVRAVDTV 10.05,
GWLRRLDG 9.05, LRVQIEEA 9.91, LPGKDSK 6.8, LERQIDDQ 13.83,
FTRAIDSA 11.18, ILVDRQID 7.99, QRAIDGDT 11.85, YRILRQIEGL 6.56,
FYREVDGI 6.76, FTDREIDL 9.78, FLVARKID 11, AIDVSSS 2.44,
GRAIHAEG 2.57, LRHYRIDS 13.19, WILRLNID 9.24, ILKFRKID 11.9,
GQDTNFEK 2.46, LDRLLDG 8.93, HGGFLNQT 2.48, RSLNRRVD 8.35,
YARQIDGY 5.9, PVIKRKIEPLEV 7.49, IPPGKALK 5.58, IRPLIDLS 6.67,
LERAQIDD 14.18, LTREEIDGV 9.48, VLRAVDDY 9.46, IRALDSDLQT 9.89,
DHSHRRID 7.15, IREEIDG 10.07, FLERTQID 11.5, YSAVHQFH 2.34,
RLNRLIE 8.03, LRSLIDEL 9.73, GDHQHFSG 1.83, IREEIDGV 9.28,
LWLFRRVD 9, LQREIEWQ 9.12, QWHIRQIE 9.99, LIVRRIES 10.23,
LNRGEIDGV 9.23, AHLRIIDG 8.72, ILKYRELD 9, QRIEIDST 9.75,
IRLIEDGRGS 10.25, TIRRIEGF 7.02, GRSIDF 5.74, LWRAIEN 10.25,
FLRQLNID 9.48, AIRS VDVG 8.84, LRVVETDG 8.14, DQWRKIDH 9.01,
FRKVDVDEY 8.2, LRASIDNQ 11.39, LFREQVDQGP 9.88, SYRAIDY 7.54,
DQDTLKGLL 2.84, IYRKLDAS 10.74, LIRFIEE 8.3, IIRLLESAGP 8.07,
IYGLRHID 9.06, KLRREVE 10.22, VLQREVDH 10.26, IRLWIDNG 8.51,
RLRLVDAD 7.3, LKQRLHID 11.87, RGIKEHVIQN 2.86, LVFRKVDSLS 10,
LRQVDVTSF 8.51, LATAGDKP 6.05, QRRVD 9.52, WITRNIDP 12.57,
LRNRIDQAS 12.03, EIRRLIE 8.19, RIREVEPI 8.58, FSTRKIDLV 12.02,
VWREIDIA 7.71, IRNIDQYV 9.84, IRKPIDNT 9.93, LLHRAIE 14.02,
LREVIEIEDAS 9.36, SIRLVDSL 8, LARAIEPEV 9.77, LRRINGDT 9.74,
IRQQIDYK 12.94, EAIRKIES 8.12, LRHLD 11.07, GPGKAEIAQK 5.72,
TRLIDLPG 7.66, QREIETSA 12.04, VLARLVDP 9.43, LERKIESLEE 12.23,
LRLVDGQNS 10.03, LDHRALDPA 9.21, LYRKVEGW 10.8, ELRQIDK 13.94,
HDLREIEA 9.5, LNRVKIDGV 9.41, KPGKTEIQKS 5.51, LDRRVEGS 8.19,
GREHHILP 2.4, LWPSRDID 6.14, VRLVDPE* 7.66, VLRLTDVG 8.32,
LREVNDNV 9.81, WLYHRLVD 8.93, LRQIYDQL 9, FSLRRHVD 10.28,
SPLRLVDG 8.09, IARKLESNGE 6.95, RPGKLESQKV 5.13, LRKLFD 9.61,
YHIRVIDS 9.07, LRLVDGHTSDI 10.12, IRQQIEWP 6.35, IQTRIIDP 7.88,
LIARSIDQ 13.85, IRNLIEQA 10.47, IDIKRTIE 6.37, WKPIRRIE 7.29,
RHRHIHQH 2.33, IRRKIENQ 10.68, VLRSLID 8.87, FTLPRKIE 9.82,
YRRDSRHV 2.32, YREITDTV 7.45, LVRSVDGSS 10.3, GFREIELS 7.38,
THREIDS 11.57, LKREEIDGV 8.8, VRQIDLS 8.7, SIRQIEVG 9.55,
LARAIESE 12.06, LARQVDGDNS 10.88, LTRKVEEN 11.61, IKGRLIDQ 12.18,
QRAVD 10.76, TQRAIDG 11.76, LRKVGEE 8.92, DKHLRRLD 9.65,
FYSREVDVS# 7.53, VSLRKVID 9.04, QRAIDGIT 10.75, LRAVDIPGLK 9.61,
AYRLIDNG 7.95, NNLRLKID 11.12, LRKISSDL 8.46, IARDIDEN 10.81,
LRNIDNPAL 11.93, AIRKNIE 9.56, VRKIEPVI 8.68, YNRRLIDA 9.14,
LDRQLDLT 8.34, HIRKQIVDQE 8.76, RTRLIDG 9.36, LDNIRKVD 9.3,
YIRQHRIDT 10.35, DLFRHVD 8.63, ALRDEIDP 8.73, QKHIRAID 11.63,
LSRLLDPV 8.96, DQVSREID 8.63, FGREVDAEY 9.29, IIRLLESV 7.04,
QRAIDGLT 10.55, LREIYTDY 5.57, VIARDIDW 6.46, AKIRHHID 8.74,
ILYRHRIE 9.64, LRDIDDFW 7.86, LWRRVVDA 7.71, KDLRHIDE 10.8,
PGKWLKSD 6.08, LDDRRVD 6.16, LRDVEDGE 9.03, LNRKLEDG 9.24,
IRKLHDE 6.88, DLDQSRHH 2.29, QRQIDSDY 9.25, IRVRIEED 8.74,
DLRKQVEE 9.3, WLLKRKLED 9.68, IRAIQDLI 9.43, ALRRNIDQ 13.36,
VRLIDYQE 7.4, AKAREID 8.9, IIRLLET 8.08, TQLRRHIDL 10.25,
QDRKRIDI 7.15, YQTRLIDD 9.42, LIRELEPL 7.22, GIGVSHVQ 2.34,
ISWNRAID 8.81, WLREVEFE 8.91, RWLRKIET 11.31, LQRSVDDTS 12.48,
DLLGRDIDI 9.63, ISRKIEPS 11.18, VVREVDG* 6.42, TQRAIDGV 10.86,
YQRKIESEE 10.37, DSKHSVSFQ 2.63, LARVQHID 9.81, LRSLDVQF 7.11,
LRAIYDEV 7.76, KLLRLVDNG 11.34, LVSRAIDLS 9.36, GADQNSNF 2.78,
ADYKPHVR 2.05, LRITKIDL 9.52, FGKLREIE 11.21, LREILSDT 8.05,
IYRHKVDD 12.1, KRLRELDE 7.36, LRESIETD 8.92, IRKLLDI 8.54,
HFRRQIDE 12.41, PWGKQQTK 5.59, FLQRLIDT 11.4, HGLRHQIE 10.97,
YLRDLDSK 7.01, FVGKELKS 10.12, IRYIDNQVV 8.91, FREKIDNS 10.54,
VREIEPWT 5.63, IRGPKIDD 8.12, FRYEIDTP 7.33, GSDNATQY 2.6,
AGIRLLDQP 6.57, LFRSIEIP 8.9, GTETRHLH 2.55, DYEPRKID 7.63,
YQLRKAID 11.54, FRWKIDEL 6.53, ITRDIDKN 12.98, IVTRLRID 7.03,
IRSNIDTL 11.02, DLYYRAIE 8.11, YFRPRQID 7.22, HLRGLVD 8.54,
IALRTNID 9.31, IIVLRLVD 7.28, LYREFD 6.02, PRGKESKH 8.12,
GRSIDDIE 7.8, LARAIESEV 9.95, WNLLRELDG 9.64, QLWRQIDH 12.27,
IYREQVDP 8.38, KIIQRLVE 8.36, KLDRLIE 9.79, DIRYIDKF 8.7,
EIRRIDL 6.57, HGNTREID 9.24, GYDYKPLH 1.95, IRLLESAKPE 8.82,
LRRTDVDL 7.33, YNPYRKID 7.74, DDTIRYL 2.23, WHLRAIID 8.08,
KANLRLVDG 10.69, IRKIHEYS 7.71, LRDLDLQQ 6.85, GYLRYIDS 9.67,
QREIKDEA 12.9, ARIHRAVD 9.68, DDIRAFID 5.81, GTLRAVDP 11.08,
PGKFLKSD 6.32, YFSHRLID 9.12, WQRHKIDE 13.26, WDSKRRID 6.52,
KAREIDES 10.44, PGNEQKGI 5.52, WIILRRVE 10.02, INREKIDGV 10.12,
GIADIHRL 2.14, LRGVDDSYPP 7.52, EPKSAEPKPAES# 4.92, FREIEKVT 9.91,
LRLVDGQIS 7.75, KDSFQNQT 2.48, LKRRIDPH 11.82, GTDHHLTQ 1.74,
DLRKIDRA 10.71, AIRSIVDS 7.65, NPGDKDTKIAKR 6.1, LLRLLDP 8.29,
YLRIKQIE 8.72, IYRSQVDV 10.7, RLARLVDN 9.31, LGYVNHHI 1.47,
DHVNREID 8.61, IRKIPFDY 6.06, LRINIDFH 10.03, AIRAIWDS 5.68,
KLAREIES 9.47, HLERKIYD 8.88, EPKSAEPKPAEP# 5.03, LQRLLDE 8.88,
NRKIDDG 10.74, LRLFD 7.84, ILREIGES 8.74, PGKVQKEF 5.3,
IDKGIHIG 2.48, ARQIDESP 8.96, LFQIRSVD 9.25, TIRNIDS 11.23,
ARLRLLE 7, LRAADLDV 7.1, LNRLIEK 10.6, LARELDFTE 8.74, WDPVRRID 3.93,
FGRAIDF 7.2, DYLQRVKVD 6.24, TLSREIE 10.79, YREVD 7.37,
TLRYIRID 6.87, DLRAFDPL 6.07, IRQFIDES 9.32, HLRNAIDT 12.61,
LRYEIKDIHV 8.99, DRLTQRAIE 8.04, RRLRKVD 9.93, DGIVRQVD 5.27,
LFGPRDID 9.82, SIVREVDL 7.61, AFLFRELD 8.17, LTTREIEQV 10.47,
HNIRDIDKALS 11.57, LRQQLDG 10.3, LNRAVDE 11.34, WFWARRID 9.07,
RNPGKELR 6.22, IQNLRQIE 10.52, QRKLDEEV 10.41, DWHGVHSL 1.85,
IRKHVDAGIA 9.64, SRLIDANP 8.23, QIERLIEAES 6.84, HLRNDIDVV 12.32,
WIGNRTID 8.87, LVLRRLD 7.65, VIHHQHV 2.21, VIRELDYE 8.06,
LSVWRDID 7.21, AELSGKAE 2.26, QLRLIGET 8.79, LRTIDGK 12.11,
YVLRKPID 6.85, RRAIDLP 8.93, HLRGQLDNLG 8.38, HNKYREID 7.95,
PGKAPKS 6.22, DLRTPQID 6.23, LVRGQEID 7.37, DIDTAAKF 2.77,
LRPIEDSV 5.54, IIRETDTP 9.25, LNGREIES 9.86, LLRAVESY 9.08,
LIRSKVDGFT 9.49, LRVRAIET 8.44, HVILRFID 7.14, DLRGREVEVLG 6.15,
WADDRHLE 2.19, WLRAIEDGNLE 9.57, RIREIELK 9.22, RIGFRYID 7.91,
GRQIDE 11.97, IRYYIDKE 9.65, DKLSRKIE 9.22, LFLRKVDG 9.82,
IRTLIDL 7.78, PGVGTKVA 5, DKKDTLES 3.2, DHLRHVE 9.7, TALRLIEA 8.69,
LRALDARPFAE 6.82, IRTLVDNA 9.43, IRQIHDE 9.71, IWRKLEVDES 7.71,
NDRYGIHI 2, GLRTDIDATS 8.3, EVLREIDR 7.86, GRLIDLS 6.85,
IFREIVED 6.72, LLRRVEL 7.21, LFKRELDPS 8.99, WTDRLLYQ 2.15,
LTQRLSIDNS 11.26, WALQRLLD 7.42, NFIFRLID 7.43, KDYSTGSSYLS 2.4,
GRLIDFV 6.15, LRRFKVED 8.48, NFREQIDI 9.93, GVRAIDQE 9.44,
YQRQIDEL 9.19, YARKIDEY 6.86, DYKYWSGI 2.04, TDRWGSGI 2.35,
GAHEYQH 2.11, LWFEREVDGH# 8.08, IIRLLESAG 7.57, AIRPQVDP 6.7,
LHIRRLVE 10.31, HLRLQIDH 11.63, LRIVEPYVT 6.67, FIRLIEYA 6.51,
SRAIDYV 6.27, RPLRLLDGP 5.72, AYILRTID 7.59, FRTIDEPL 5.76,
GAIRDIDLK 6.71, LVYRTIDP 9.87, DIRHIIDS 7.46, IRNSIDTF 8.29,
PGPREGK 5.09, YFRSQIDDL 8.1, WFRQIDSN 11.33, IREVEFSN 7.5,
EARRIDF 4.16, IYNRRLVDS 9.55, LKRYIDPG 9.35, SRQIDY 6.21,
DNDQIFAA 2.31, HIRKQVIDQE 9.1, LLARLVDS 9.49, GSDNWSGYS 1.84,
LRLLDPQ 8.36, LRKVADEL 9.05, GTHLPLAG 2.23, FFGREVDAE 8.29,
DLRPRKLD 7.01, QDRDIDIV 10.28, LRHIDGEW 6.28, IDREIEFLPS 9.37,
GRYQIDS 8.45, LRIEIDFRE# 6.55, TLKRLVDSS 10.5, IKVFREIE 6.97,
VIRLLESA 5.68, VYRQVDPI 5.97, FRLIDPYG 7, DWDQRNHH 1.82, LGRLLDE 7.9,
PWIRYIDE 5.87, SRQIDIFP 6.78, HHQLRLVE 7.7, IRLINDLG 6.7,
RLERQKIDGV 9.62, DHELKKFQ 2.43, NLVWRAID 10.03, TLRKLVDT 9.33,
ADKGYSTY 2.12, FLQRQIDP 13.67, TEPKSAEPKPAEP# 5.6, WLQWREIE 9.26,
RVFRDIDE 6.96, WLLRKLDL 8.63, PGKQTRVS 5.26, DGLVYEGRGWNFT 1.89,
NQPDREID 8.74, LKRELDQTL 7.05, QLRFIDPA 7.53, IRIWIDQP 6.4,
LHYRLVDTAS 10.66, SNIRKIFE 6.89, IRSIIETT 6.38, HLRPIDEE 7.93,
LRDWQIDF 6.59, WIRHIDEE 10.35, AILRTQVDP 8.94, WLGRSLIDS 10.17,
HIRHAIDV 9.68, FKLRQVDS 10.82, YRTLRDVD 6.56, IALRFIDV 6.53,
LRKVDGQH 10.05, DTRAIDQF 4.53, GLRRVDDFK 6.9, FTQRYRID 7.13,
VRLIEPSH 7.46, GVHVHGGY 2.79, LRRDLDA 6.77, PGKELRKRS 5.92,
FRNIDTPQ 11.27, IYLVWRRIE 7.41, LRKIHSIE 5.63, SLAAFGHI 2.13,
IRWDIDDV 7.09, RQKREIDV 10.39, LRLVDGQTSDTV 9.74, KDSTHYLG 2.33,
IGLRDVDPG 7.59, IWRIIDAQ 6.34, FPPGKHTK 5.56, YLRAILDAHS 9.65,
LRTAVDSLV 7.37, NLTRFRIDELEP 8.1, LDRAHIDN 9.94, QRAIDEDV 10.06,
DNSSQAHL 2.41, LLRELDQKE 8.53, KLHRYIDS 12.19, IFRQIIDY 5.86,
QVRAIDL 8.22, IRGIDDSI 9.25, LRENIELG 9.25, LARFRIVD 6.42,
LRRAVEVL 8.65, PPKSANKE 5.6, LRGIETYP 7.11, LRRHIDLL 7.11,
TDVQRGYW 1.89, IRHLLIDG 7.29, LKREAIDGV 8.05, VAPGKDLTK 4.34,
FRKLDEL 7.56, IRKTDDAL 9.76, IWLHRQLD 9.28, LRREVYDF 5.68,
IIRELEPGV 6.89, DHRDEKAV 2.47, SSGRDHNF 2.64, WHQRAIDD 9.43,
GTRRIDF 6.52, LRAIVEGFQP 5.66, LRDLDDTSV 8.38, ILRRITEIPE 4.64,
GSSSHHIA 2.3, GDEKGVLW 2.3, SIARLLD 7.09, IRAVDSNL 9.29,
LRALEPHSE* 8.52, FGALRELD 8.41, PGREIAQK 4.85, PGFREFLK 4.88,
YDWSRGWLS 1.71, SPLREVDF 5.8, GLFRKHIE 8.95, IVQRLIEQ 8.4,
AIRQQIES 8.89, GRSIDDA 9.63, IHYREIEY 6.96, WLRELDDH 7.09,
ILRYEIHD 7.6, KLRAEIENL 9.18, NLGRRIDNL 10.41, INPHRTID 8.29,
GGGFHV 2.15, WIRKNIDK 11.08, AHLRAYID 6.29, VLRKLDLV 7.48,
YLFRSVDAV 9.74, IQPRQIDL 7.66, LNRGKIDG 9.07, LRGRIEEL 5.7,
LARWHIDS 9.69, LRRETDANLG 7.54, FREIISDY 5.67, AHLREVET 8.28,
IARFIEGGWQG 6.62, VHKIDEPA 4.22, LYLRQKVD 8.41, IQRRLQVD 9.7,
LRIGIDNV 7.32, IKIRRRVDV 8.7, GDVTHESAS 2.48, IWRELDE 7.19,
LPRKLDS 6.72, GIRSIDFERVG 6.26, HLRAIGDGE 9.53, QNRFRSID 9.45,
LSYRNIDT 9.79, KDLSTNL# 2.38, TLKREIEK 10.58, HDFNAFHI 1.93,
TLRDIETF 8.1, IRDFDGYV 5.35, ARLRLVET 8.26, FRIRLVEA 7.42,
LGWRVIDN 7.5, LRVKIERDDLS 6.83, WLGRTIDE 9.41, TVQRYQID 6.77,
QLRKLVDLA 9.73, NLKKRAID 11.25, YIHRNIDE 10.66, HSDPASSP 2.45,
LKGPRAID 8.99, PFFLRDID 5.41, EQRLIDIS 8.2, IRPIDKTY 6.01,
NLRLLIDA 9.77, YLERRIESEI 7.61, ARLRLVDVV 8.72, WYVLRRVE 7.75,
LILRLVDADE 9.24, TYRRIDG 7.71, IRKHITDQ 7.47, FRALDGTGAS 7.84,
RIQRLIEE 7.48, DSNAGHTH 2.29, YDRQIDLT 5.03, LALRSIET 9.82,
FKVRDID 6.91, YIRRLDSD 7.21, DHLWRRVE 7.76, ILIVRAVDG 8,
LRIKIWEN 7.73, YHLRTIDV 8.02, LRAYLDGTGV 5.31, QYPGRDTK 6.48,
ERKHRHFH 2.36, LRYITDTT 7.54, LRFVDQIP 6.87, LLRENIE 8.37,
FIRQVDRP 8.74, SGQHHGV 2.41, QKRDIDVE 11.82, VREVDIAG 7.5,
LERRIDSL 7.15, LRGRIDYY 8.7, LRALLDET 8.03, GIRDVDPK 6.37,
VYREIEQV 5.59, LRRHIEDQ 9.46, GRLLDGV 6.82, GRDIDESKV 6.49,
VRLRYIES 7.24, EFREVDTP 6.53, IVRKWIDH 8.32, FIQRAVDS 10.74,
DGISKHHI 2.46, LHSREIE 10.55, LRLKVDT 9.05, SIHSKHIQ 2.45,
PGFEQKSPS 3.94, LDRKFDIE 6.55, LRWQVVDTPG 6.93, QQDSGSAF 2.1,
WLRGLDSV 7.38, DHGSWWNI 1.66, LRYIIDKN 9.68, LSRSIDAAL 8.25,
LRASVDLFTP 6.26, LRDKHLID 6.04, ALHRAVEP 9.09, WSGGLAQ 2.1,
AEPKSAEPK# 5.21, DDPVVPFQLG 2.57, LRKEISDV 6.55, WKYIRFID 8.35,
DLSSSLDHS 2.21, DISRRNLDI 4.37, DTIRRIEE 6.74, DKLRFITD 4.18,
TLREVFDN 5.54, IAYRPEID 5.36, YLRKFDVN 7.27, ILFRYHID 9.65,
LRSIDSGH 9.98, KELRLVDGE 8.01, DYEVREID 6.34, APRHGLGH 2.15,
ELRDVDG 7.1, DHDAKKAS 2.29, DLFLREIE 7.85, LVRKLDLS 7.52,
GDSEFVNR 2.22, LNREQIEGV 8.9, ELRRQVDQLT 9.25, RNIRKVDP 10.49,
LYRSIDSHTE 9.62, IRLKITDSGP 8.43, LRTSIDAY 6.07, IIRLLESAQP 6.43,
SLRLVDAL 8.37, KGYREIDQ 8.48, KLRRIDLS 9.77, QHREIDNF 7.46,
SLRSIETA 10.1, GYFRLIDV 7.77, IIVIRQVD 6.61, GIRLLENP 6.01,
IGRAIVDN 8.26, LTFREIEL 6.86, KLFTRLIE 7.47, YTLRDVDD 8.29,
VTRLIEGNE 6.02, LIRAVEIT 6.42, YLARRVESEV 7.16, LYRDIENP 6.99,
LRQQVEQL 9.04, QARQIDFP 7.21, FPGKQFKS 5.61, HDQWIHGV 2,
DHLAKRDVD 6.32, GYHHASIA 2.73, DKETLIQF 1.94, LRAIEYTI 6.25,
SIGHAVHL 1.8, LGRSVDTSS 9.46, ARLRVIDE 6.13, HLRELDLY 6.13,
ARKLIDE 8.22, ALREIVET 5.78, LRSDIDFN 7.11, DAQTQIHH 2.43,
GDILKVLNEE 2.15, EHIRDIDV 8.72, IREIDLFV 6.24, HSTREIDE 8.33,
PGKKNLKP 5.31, LWFEREVDG# 6.47, VDIYQHHF 1.79, NKHIGFHV 2.16,
IRILRDIEQY 6.61, FIRILRID 6.31, ILRTIDRP 9.57, YRIQRLIEE 7.04,
GDIGYLNH 1.86, IRQLEGEGVL 6.65, IARFIEGGWTG 5.87, ITSDRRID 7.18,
DFRLIYDG 5.74, LRSLIEQI 6.29, RQVLPAVL 0.68, PGKTAQTK 5,
YQDARQID 8.01, DTGWWPLN 1.9, QLRAVEFG 6.63, LRGLDGNGTG 7.42,
PGGKQTRP 4.69, LYTARQVD 7.7, YEHRLID 6.98, VEREIDG 7.66,
EPGKHSK 6.74, IRDIENWV 5.43, TQRAIDNL 8.82, IREIRDVW 5.57,
DGQVQRHG 2.48, LRLVDGQTSDI 8.05, YRLQRNIE 7.76, ARKIDPIA 9.76,
RVQRQRID 6.04, QDRDRSID 7.17, LRLEIRDLEE 8.19, IRSLDKFGD 5.72,
YREIDWDN 4.69, LRLSVDSV 7.93, LQVERDID 7.74, FKRYEIDW 4.75,
VRQIDAFG 4.67, NKRQRAID 9.39, ARKQIDFV 7.21, LRRLDTSLGS 6.98,
LNRGKIDGV 8.93, QPRSIDAT 8.92, LRFQVTDLDE 6.85, LRLVGEGPSV 6.54,
IRLLETI 6.13, IFTRFNID 7.26, AQIRKLTDLE 6.84, DLIKRALDF 5.6,
DQFRQHID 7.56, IRRVLDGG 6.37, PGRENK 6.56, WIRWAIDV 5.47,
QILQRDVD 8.02, IKIRRQVDINP 11.05, TFIQRVID 7.02, FRVQIDGE 6.76,
IYRRLDG 7.88, FTNGTHHL 2.05, LFRSHIDT 10.62, IQRWIDPE 5.5,
LRRRVEG 9.29, LRLTDDLI 8.75, LRLVDGQTSDV 8.62, RREIDYNF 5.01,
RGESKIVES 2.24, KLEVVNHT 2.07, QRKEVDLDG 8.71, IIGRLLEGS 6.29,
AYGWANAL 1.9, LRQQWIDV 5.44, ERPRRID 4.91, LRGWIDSQ 7.44,
WTGVAQSGDSYAS 2.06, DDKHNYIV 2.27, LYREQLD 5.79, ALRELIEE 7.19,
PGYKDYTK 4.68, LRATDRID 5.9, HAKIRLLD 7.92, ILRSIHDS 8.85,
NTVLRLIE 6.59, IRDLTDDP 7.74, DSRLIDAL 5.64, DQEPRRID 6.17,
FRLVDDQI 6.55, LRLVDGQSS 8.44, HANRAIDV 8.46, LWFEREVD# 6.46,
DLRSLEPEGAAE 7.64, LHRKLDNS 9.19, DFGRELD 6.03, IRRLDSNF 5.71,
IRAILDQF 6.6, WQEWRQID 6.75, DGAKD 2.25, DDSERLSGS 2.7,
ALARQIEE 8.18, FLLRAIEE 9.12, NPGKAHIK 5.67, LFSNRYID 7.92,
LARDIHHI 1.63, SLERRIDNL 7.76, APAVGGFGS# 3.6, PGKGANKN 5.19,
ELRRVDFA 4.65, LPRNIDH 7.14, WIRRFNIE 8.13, IRRLVDTHG 8.41,
PGTQTKPD 4.24, LRNVDDAV 9.1, KWLRNIDY 7.51, QRKIDTIE 9.64,
PGKLYNKE 4.87, TPRLIDG 5.67, LRAIDKYI 10, DIVRLLDQPS 6.16,
TRLIDEPQ 7.94, KGVRWQID 7.02, LQRVIDSQ 8.59, LRLKVEHE 8.89,
QHYHTVGA 2.61, LTRTIDPL 6.12, IRQVDVTI 6.61, DLIRFIEE 6.16,
SVSGWHVN 2.36, IRS VDEIV 7.82, KILRQSID 10.04, LQRLFD 6.14,
LFVRYIDQ 7.79, LRDITDDW 7, YLHVWRRVD 7.19, LHRTIET 9.67,
TSWREIDF 5.68, IASYRTID 6.81, EFAHHKP 2.36, KIRLDIDV 5.5,
ARRAIDAF 7.35, KFREIEVI 7.16, IRTLIDQK 9.35, FLQRFIDP 10.53,
VSHREIDS 9.3, TDRNHIKH 2.13, IRRRVDIN 8.29, LRQKILESGGV 6.56,
GDPGHYRF 1.87, LAPRRIE 6.36, ELRRQVDQL 8.71, IDLRQVEV 6.91,
GDRLIDFT 6.63, GQRRIDFV 7.21, IRWVEEPL 4.29, LRDEIEEL 8.55,
YTLRALDPDS 6.97, PLRLIDG 3.73, LLRKVYDA 7.62, HDRYDWYN 1.83,
IRAIDRDS 7.63, LGRLLDN 7.65, NGRLIDS 9.68, DNHSPITL 2.34,
VLRGLIDY 6.52, LRQLIDHW 6.59, IRGVDIDNPYFNF 5.93, KRAKLREIE 9.41,
FRSLIDDT 7.76, DLRVVED 5.55, PGKRIQKS 4.81, DPSRKIDG 6.19,
FHIGPEQH 1.87, HPGKIDFK 5.97, HLKYRFID 8.91, IIRLLENS 7,
LFRQVDQW 6.35, VIRIQIEP 5.2, DDFIHTQP 2.32, WTRWKIDV 8.06,
YFRWNIDE 8.59, IRDILDGQ 5.08, NLYRAIEQ 9.55, LRAFIDEF 6.32,
DQRSENID 6.35, APIRQIDV 6.22, HILRAIYD 6.93, GPLRLVDGQTS 6.59,
YPGKFVKE 6.73, LRKLWIEGIE 4.66, FVHHVVNE 2.18, IPREIEFE 5.17,
SRKIDT 10.14, IRDVEKPP 8.6, AITRFIEGG 5.3, IRNWIDQD 6.8,
IRLERIDS 7.04, SLRRDVDES 8.28, QHISDHLSRSQL 2.16, QREIDGNF 7.34,
AEPKSAEPKPAES# 4.61, FDREIDHL 6.25, PGKLPKG 4.86, LQQWRDIE 6.27,
LRNIEKVEV 8.69, SLRGKIEDE 9.5, DTQSNIVS 2.4, DLRIVEAA 4.08,
YHVRLIEP 6.44, QRAVDVDDG 8.71, WVDPKQFV 1.85, YFNRELD 6.84,
DLIYRTVD 5.18, LDRFKVDT 6.56, IRAKKIEE 8.46, HNPHRQID 7.24,
SFNHRHL 2.15, FLRSISDDA 7.74, KQLRVLIDS 9.06, LRQLDFVEEV# 7.31,
NKHREIDV 9.21, IREVQDYV 7.12, ALRRQNVD 7.99, GLDVKNV 2.33,
QKLRREVE 7.88, ILRELDVSYV 6.47, GLGRYQVD 6.04, IWRRLVEG 6.83,
LRIAIGDSP 6.32, DIIREVEE 7.1, DDPYFKTA 2.67, LSLRKLED 7.96,
FRLIHDQP 9.49, IRGAIDGQ 9.71, ADYKHYHS 2.17, FRHVD 7.65,
HLEYRLID 6.94, RWTRLIDG 5.97, IQWFRQIE 7.47, QDYKFTFA 1.69,
LRVTDPYNDLV 4.65, LERWLIDS 7.29, LSLLRALDN 7.85, LRWIDGQW 5.31,
TIRLLDV 6.7, LREQILDLS 7.51, GRLVDGIG 6.42, LNRVEIDGV 6.09,
SVLKRRIE 7.7, NDRARIDI 2.72, QREIEQL 8.57, HIRRAIDK 10.25,
GDGSLRWP 2.12, DLRWIDGQ 6.66, IIREVHDA 8.36, LNRDVDLA 8.89,
KLNRLVE 9.08, IIRLINDNFQ 7.33, FIRRIVDT 6.65, IRLLEEAL 6.79,
LNYRLVDT 8.25, GPRRIDF 4.41, IWRVERID 4.9, HWLRATDP 9.87,
KLARAIEP 8.63, DAQDQQFH 2.1, VSHYNETQ 2.16, WYEHRLID 6.56,
FERLIDVG 7.26, FQQRELDY 6.03, LARALVDE 7.18, IIRLLEA 6.84, SLRLLDS 8,
LIERHIDT 8.44, LQRRPNVD 7.02, DIRKLFDL 4.96, WLVRQIDI 4.71,
LNFRYIDG 9.39, LRNLISDSL 6.34, FFDPQLVQ 2.05, IDRTVIDN 4.04,
RLRLWVD 5.53, FQRRIDEI 5.71, LIRGEIEY 6.37, QRDLIDDAT 9.28,
DFRSRFID 4.97, WIRKAIEY 8.72, IYRAVDNW 7.27, DHFHGGGI 2.14,
IERREGIDVS 6.78, IRSIRDVV 6.06, DRLIHHIQ 1.85, LIRSAQEIDE 5.48,
FHREIEGSQV 6.71, HQRFQIDN 10.34, IRSKVELEV 7.6, DTDAHGYY 1.92,
LRDNIDNH 8.94, GRDVD 5.86, IREFDGPL 4.17, YDKSHGDP 2.18,
TIRAIFDT 5.95, YRKLIDQP 7.83, GRVKIDEVS 8.62, IYRRIDAK 9.43,
QRKVIDEA 9.42, ALVSRARIDAQ 6.92, FLFPRSIDV 8.82, YRQIDDS 7.19,
IREVEDSK 8.34, SRSIDIGY 6.08, WIHAREIE 8.5, IREIHEGA 7.76,
QRLEVDYSI 6.92, HLSRNIDF 6.71, DRITGRAIEV 3.89, LRQYDSDEP 7.28,
LQAGNATEV 2.47, KGREIDFE 7.59, LERRIDTL 7.26, DYISIGHQSTNS 2.53,
IHRVIDQT 8.87, FRLVDEG* 8.03, HFRALIDE 8.39, IWRPIEID 4.88,
SDHKGIHH 1.52, TLRIHIDL 5.89, QIRNQIEY 6.66, IKRDIEEF 6.36,
ALRGEIETV 5.89, SLINRHID 6.95, QRELDEATES 7.51, EHIRFIDQ 6.57,
QNRIQIDPV 8.93, YIDKAANV 2.08, PGLQQKP 4, LARRIENL 6.34,
QLYRNIEP 8.15, PLKRHLID 6.45, GIRSTDIDES 6.74, DGVQWQAI 2.41,
LRHITDST 7.88, IITRVIDT 6.12, LLRATDGW 6, LRKTIEVH 8.63,
IRLVESARPE 6.7, RVSPYSIFLQE# 5.29, DTRKEIDA 5.46, ARANRQID 6.83,
NLRGELID 5.3, QRHQIVGH 2, FREVEEL 6.41, LRLVDGQTSDIVS 7.95,
GIRFLIEG 4.29, GDRAIDTV 6.59, DTGWKFAI 2.14, FRSQIDEF 6.18,
IRKVEFQY 7.77, LRSEIEKA 7.69, AQRAIDSQ 8.3, GNDGAKGDAG 2.12,
LRELLDQS 6.86, IRTIELDG 5.75, PIGREYQK 5.08, AVLRLTDVG 5.88,
IERQKIDK 7.07, FKRKIDDH 8.09, LLSRLHIE 6.9, IARDLIDFD 7.68,
ILREHRVDDS 6.8, VRKVDWEG* 4.94, YLRQLDVL 6.42, IRELLDS 5.96,
YDNKTLA 2.06, DLRQFDGI 4.73, LRVLDSFGTEP 5.17, QWTEREID 7.22,
VGRLIEG 6.15, LTPLDNASLT 5.98, DHQDKKNI 2.25, NLFRDKID 6.55,
IKRQLDSV 9.27, VAFRQKID 6.23, WLKRKFID 7.33, IARKLEDVF 5.91,
TLRQLDL 6.83, NTLPRRVD 6.72, LLRGQVEF 5.52, LRQATDGF 5.27,
WLSRAIEA 9.11, IRKELDEE 7.16, IWRIRIDL 5.53, IVRVLRID 5.09,
LRLVDGQTSN 7.37, LRWLDSTP 6.46, ILDRLLDG 7.13, PGKALRPV 4.84,
LRHVE 8.5, IREIGDLW 5.25, IQKIRFIE 5.96, NFTRQIDW 5.4, VRYIDIVG 4.96,
LRQGLLDTS 4.14, EILRRSVDTS 7.59, VRRIDYIG 6.66, LKSRRVDFET 5.67,
DLRQQLREITE 3.76, YVQRAIEG 6.83, LRLVDGQTSDIV 7.75, DEKFIHYA 1.87,
LEREIEEF 9.24, LRAYLDGTS 5.48, GLRSVDLQ 7.18, LSRAIDARS 8.62,
DDSSLKGL 3.02, LRPLVNID 5.15, IRLTIDTT 5.61, QTQKRLID 9.09,
DSDQQTLY 2.65, TDGLRKVD 6.78, QWRKLD 8.16, FRETDEVS 6.43,
GDHEGASL 1.68, LTGQRIID 6.33, KSQLLREIE 8.14, VRNIDGS 6.65,
IQNRIQIDAV 7.81, NLTRQIID 6.83, YDGQKDRV 2.38, IIRLLEN 5.98,
YLKFRNID 7.23, DLDRKVSDLENE 4.69, ALGRTIDL 5.94, DLRDVETL 6.24,
IRDVELAE 7.77, DFSSSGDG 2.18, LRIARIEE 6.06, DWDHLQLEG 1.65,
DQRDYDDP 4.03, FKREKIDA 6.79, LQIRSVDNG 8.36, IRHVDPGD 7.75,
LKREEVDGVK 6.8, QLRRHIDLL 5.59, WPFFREVD 4.22, TTNLRSID 9.29,
IFRAVEAI 6.53, THSIGNQI 2.49, EIWRDIDF 5.96, DDLRSVEE 4.01,
IRIIEEFT 5.24, TIFRHIDS 8.75, NYDSITPNGS 2.16, LRQTDLAGSS 8.3,
HKYYHDG 2.02, RRAIDAV 7.56, GIFHAKLH 2.15, TVLRFHID 7.78,
GRREIE 6.51, ILRLLENA 6.26, IRLVDIAAQNP 6.46, RLYKTSWR 0.7,
IRAFDEVP 5.56, IDRIIKDE 4.16, DVLRQFD 5.13, QREVDKDK 8.18,
HWQRRIDS 8.08, LNRVIEKPNE 7.69, IDTIITYN 1.82, DVRLIDAQ 5.84,
FDGNRTGI 2.16, LANRRAIE 8.22, IIRQIELK 8.23, IRSLLIDG 6.03,
LNREIQDN 7.22, LRKVEEHS 7.92, IRLVDILGQNP 5.92, ALRGIDEE 8.22,
LDALRRIEAG 6.86, IRLLDHSP 7.63, GVITLIHG 1.83, LRDFSNID 5.02,
EKDIAAYR 1.91, FDRLRIVD 4.33, IRNILDLT 6.07, DYSIWVQY 1.7,
QKHRAIDI 9.26, DFHEKQYQ 2.14, KLNRFIE 7.02, LRKGEIESQ 6.53,
ILHGRLVDS 9.02, GIDRWQGI 2.04, FARELDS 6.82, HYLDREVVD 5.16,
IRQVEEVFS 6.69, DIRRTLDA 3.83, IHSRRSIE 8.79, GDLRQYDS 2.63,
QRDRSEID 6.4, LRLYDSAV 5.46, DSQLLAVT 2.1, VLQRLVDIG 7.1,
VGKDLKGD 6.07, GRKIESDI 7.7, NYIREIEE 8.08, LRAVIEYS 4.82,
AYIHVHHA 2.89, HWHNRRID 6.2, RRAIDIPS 7.68, DNPDKFAW 2.04,
NNLGRRIE 8.12, IRWHQGTL 2.08, SYLRKIVE 6.4, TGARRIDF 5.43,
AYLRQVEG 6.94, TIREIPDL 5.62, YYLRWKVD 7.51, IAGFRTID 6.72,
GIDRFHV 2.08, GDRHFDQV 2.24, TLNRLVDE 7.53, DGYAHG 1.75,
KIGETLG 2.07, QPGTQVK 5.17, LHRVIEDG 7.35, ILRFVETD 5.95,
LQRDLDSL 5.95, RDHRLNTL 2.1, DLLRLIDYNK 8.38, IDKRHIET 3.64,
LDRRNLDN 6.46, WQPWRLID 4.8, DIERIIDD 5.37, WIRDIDWK 6.59,
DTLRNSID 5.5, IKLRRTIE 8.08, LRVLLDSPV 5.78, LRKEVEHE 7.35,
PGTAQKGY 4.26, LRGGRQIE 7.72, ANEQRRID 5.64, GDRRIDFL 3.55,
EALIRLIE 5.82, DVFKLGNI 1.91, IRRGIETV 7.12, DGKDGLL 2.52,
FKHRHETI 2.63, HRLPRRIE 6.98, ERINRKLD 5.95, NAQDPHVG 2.09,
KQYREVDV 6.86, SWDHVKLH 1.97, YGNFRAID 8.26, KIKRHIDG 8.98,
PLGRWEVK 3.73, PGRQQLKV 3.8, LRQKILESGG 5.83, LIRLIFDP 5.57,
AVHTLLSS 2.09, YQQRGEID 7.54, VWQRFEID 4.86, GSSGHASTS 2.14,
LTRGLESGIITS 2.3, DGANHVKN 2.41, DYFSRKLD 4.89, LVRASIDLGS 6.42,
SQGIRSID 8.26, ALVSRARIDA 5.73, IRWLTDEA 6.96, GYDRHGSI 1.85,
DFTRQFID 4.05, VYQRLIDK 9.05, DGAHPKDR 2.13, HQKSRQID 8.94,
LPTAREVD 4.95, APSGGQYTGS 1.88, KGILYRAIE 7.83, LKRETDENLK 5.32,
QRAIDQIT 8.1, QLRWPEID 4.38, AAAGDKPSP# 4.21, IRDIDQHD 9.51,
LTWRPKID 6.55, SLGRRVDG 7.26, QHLRRVDAPVLES 7.37, TDGYPHRS 2.19,
HLFRAVEPG 7.81, YQRSNIDG 7.8, LNREKIEGV 6.92, LLRKQVWD 4.86,
FRNNIDE 9.3, LRGIIDQIQ 6.93, ILRRFVDTSS 8.48, IRLKLDHD 6.53,
EHQRFQID 5.94, TIQKQLHH 2.31, NFRSIDPQ 8.46, KDLAGSD 1.92,
QFFLRYID 6.5, FTRGEIDD 6.69, SLLRKLE 7.28, NSRKIDAL 8.26,
TSRAIDLP 7.01, DSFHREIEGS 4.19, GRLLD 6.32, IRVIEDVE 6.17,
KIIRQVE 8.06, IHAREIFD 4.36, GIYRWEVD 4.63, LDFQFTNA 2,
LHHVGSLS 1.86, KGALRAIE 9.49, LRTWYRID 5.48, PGTEQKGR 5.28,
LRAFDEEGA 6.72, LLRFVDDI 5.62, IRRELDLG 5.23, IQRGDIDALISS 7.84,
ILVRNIDLV 6.97, IQIRLIEW 4.26, LRTRLVES 6.79, VRSIEGAE 5.86,
LYRHDIDS 7.91, IRSLDFNP 5.44, SFRKVDPY 7.02, PQLRTDID 4.23,
NYIRILID 6.05, IQHRIIDY 5.44, KDTPAVFH 2.12, HPGKRQKE 6.27,
IGLAYVN 1.99, YFRDLIDP 6.01, PGHKWKEVR 4.12, LRRSVDASS 8.74,
IRKADVEG 7.23, LPRAVID 5.06, VDRQGASI 2.03, IRLLESFET 5.24,
FSIRKLDP 7.12, KWLARAVD 6.74, SQLRYLID 5.98, LRNVDSVV 8.29,
ITKREVEDDLG 7.97, IREADIDG 7.01, DLRQYDADEP 5.62, LVRLLEGEGV 5.07,
IHRVVDPQ 6.78, DQRVSLIDDEPS 5.67, RDFAPPG 1.97, PGKPEGRP 3.51,
AFEWRRID 5.51, DLRQYDTDEP 5.83, LPRRIEIA 4.38, QIRQEIENS 8.23,
LLRAVESYL 5.94, LTRLLDPYP 5.81, DYQQSQFSD 2.37, IWRAIADL 4.6,
YLRKNFDQEPLG 6.21, TLTRIRKWIE 5.97, VLRLYD 5.51, IRRELDK 6.71,
LTRIEIDP 3.95, PGTATKES 4.66, ILIRTIDH 7.97, IRRKGIDA 6.17,
WTFIRLVD 6.04, LVRRLDAS 6.36, HDNGSENK 2.38, LRSFDPQF 6.58,
VGREVDIA 5.21, QYLRQLDG 8.37, DKWILSET 2.36, TGILNRLIE 6.21,
LDRATDIV 5.23, GGSDSTT 2.22, FRAIEDPL 5.53, GRLVDSIG 6.28,
LRPVIDSP 5.98, DLRSADDL 3, DWRAIDIS 3.41, WTVTRQID 6.75,
KIRNIELP 8.17, LLRFRYVD 7.18, FRRAIETG 7.08, VDLDKINH 2.09,
YLLQRAVEV 6.82, NREKIDEV 8.3, LQRQIADT 9.32, GIRLLEE 5.06,
LRQADFEA 6.84, FLRSVETF 6.63, YRKIDQTD 6.46, AHPKVWIH 1.78,
NYRDIDLG 5.45, RDSNHVG 1.7, GEDRKPSN 2.23, GFHRHQVD 5.33,
IKRLIYEN 6.22, LRDVDKAH 6.32, LTRGFESGIITS 1.79, RLHRYIEG 7.51,
QQRDIEYG 5.9, QQIRKLE 6.6, IHAREIFDS 5.05, WAQRIIDS 5.94,
VRALIDN 6.35, DGYSFFWQ 1.6, WARYQIDL 5.36, DYKEALLIPAK 2.31,
IARKVELA 6.94, FWTTREVD 6.2, IRQEIEITGT 7.3, HRDQGSSAL 2.39,
DRYQRELD 4.65, LRQKIDKF 6.56, LLSRSIEI 7.27, YDGNGKL 2.37,
SQIVRHIN 2.02, REDVDKRAR 2, LYRWQTDV 6.2, IGHWVIH 1.85,
FRKLDGIS 6.95, WTGHGTLQ 2.46, VRWKVDGN 4.78, IDLRLRLD 4.85,
PGSREPK 4.79, TDQREHLQ 2.1, LREEIEE 7.25, SFLRRIEY 6.53,
IRGRKLETEV 5.18, QREIHDE 7.7, LRQADDAP 5.75, PDGKQVRG 4.26,
IKRETDSE 6.79, DEVLHGLQ 2.2, NLRYIDGA 8.23, GVIRLLDP 6.28,
TRRSIDQT 7.76, QDPAHSG 2.06, RITRTIDY 6.72, SARRIDP 7.5,
QRSIDQQF 6.96, LRARIEQA 7.31, APGSTAPP 2.17, DVRKLDFPS 4.87,
LRERIDRAE 6.61, IELRKLEA 5.51, LRQLDLGSSILTE 7.1, SIRLLDQ 6.39,
LRGVDLLQ 6.04, PRLIDGS 3.62, WGHDVNIK 2.34, HGPIVIIH 1.51,
GANRDLQDNKE 2.28, LNLRALDD 7.25, LLQRQLVD 5.88, RARRLIE 6.28,
QLRQAIEES 8.68, LRAPIEFS 3.77, HGIRLLE 4.9, INPGRQIK 4.21,
DTIRAVVD 3.36, HVREVDFS 5.11, FDRPSAQN 1.78, LRRVLDELT 5.43,
LRLYDVT 5.95, LRHVNIDHL 6.98, GDPAHLGLS 2.15, AIIGHSLG 1.92,
QPGKLIKP 3.95, IRKVDEGR 6.66, WKIPRQVD 6.07, IREADITPA 6.37,
ERKQID 6.44, DRDREIDN 5.24, LRSIHDDG 8.18, LRKSEIEY 5.37,
WNLYRRLD 7.09, GANDYKWQ 1.79, DLWRLIGD 4.67, VYHAQSIS 2.31,
DIERNIDV 7.02, DIRKQVVDQE 4.11, NDRGNVSAQG 2.17, LRLADTTE 7.34,
GIGRDLDI 3.86, LSRRVDNS 7.49, QFLRKRIEA 7.11, IRKLFDL 5.13,
FGPRSIDPT 7.36, WWIRHLIE 5.62, LRSLLDLENG 5.58, SLIGQSLS 2.2,
GRLIELS 4.49, ALVSRARIDV 5.85, IRLFDLPA 5.79, LREFDSIT 5.66,
ILQREIIE 6.35, GVRLLDG 5.41, IIRLLEGAKP 6.37, LRAAIELP 5.99,
RLDRRHIE 6.97, NHREIDS 8.42, YQRGLIDV 6.65, GNHSE 2.17, HFETRRID 6,
LREFIENT 5.97, FREVDWFE 4.46, EIARRQLD 4.23, ARAIDFVD 4.88,
LRHPIDRP 4.82, DTRYIDVA 4.02, PGTENQKQ 4.16, PRLRLVDA 4.97,
IRRRVDINPG 8.01, NQRLIDEQ 9.01, GIDGRINF 2.22, QRKLD 6.9,
LGREKIEG 4.95, VIRYVDNS 5.54, GDWRWQGV 2.03, ILRHKTDE 8.29,
KLERQKIEGVNLE 6.08, DYSAVGYS 2.32, VFRELEPAV 4.49, DKSLLHKVSDTG 2.53,
HNEPREID 6.57, YFERLIDS 7.05, LRQQTDVI 7.36, WFRRIDDK 6.65,
QRLLDDTS 7.55, TRDHFSPL 1.94, IRLIDVWV 4.54, TREVDDT# 7.32,
IGKPEIKIL 5.62, PGVEQKIN 3.82, ALVSRARID 5.67, LRELTDSH 6.63,
YLPRVRID 4.81, LRSDRFID 4.57, QNRIQIDP 8.29, GRLVDGVVS 6.03,
LPERKVDD 6.38, DLRINIDR 6.76, IFVRAVDGG 6.78, SREIDAQS 7.32,
ELNRLIE 7, QIYRFEVD 3.92, IDRNIDYR 4.55, NRIRILIENGV 4.2,
LRGLIDYY 5.27, LRRLADAV 5.88, IDRNIRQL 1.99, AWDIHIYH 1.72,
QRLLDASV 7.52, ARDEIDAPN 5.28, LARLLEGDE 6.9, GGTSHAFS 2.2,
NLRQGVDADINGL 6.62, HLRHKIHE 4.88, DTDYRSLEY 2.01, HQDWSHAA 2.06,
QVRQIDHI 7.39, DLRQYDSDEP 5.71, IRDVDEQV 8.4, VYQRDRID 4.46,
DLQRELEIP 5.14, LRKENVDG 5.79, GSWEGHHR 1.85, LDHHFGTN 2.14,
LRQVNETWT 4.82, RVATWFNQPAR 0.74, LALRNIE 6.38, YKDFRLIE 4.94,
AEKRLRAIE 7.27, IRYRIDSK 7.86, AQPHYVQI 1.92, QGWRDQID 4.87,
GTRSIDVDES 7.84, YINRQAID 6.92, IYRFVEVD 3.97, DELLRRVDAE 5.69,
GLRIWIDQ 5.29, VHQLKHEQ 2.16, SLEQRSID 8.74, FQRIKIDW 5.92,
GPIRKIIE 5 44, SRLRHIEA 7.43, IVLDRLIE 6.02, RRGYGDIY 1.75,
QTVRWEID 4.51, STQWLSHI 1.85, IGPTRLID 5.13, HAENRKID 6.91,
DVRHIEGA 2.72, WLRLEIID 4.19, QSLRAVDPLG 7.64, VIRLLESV 4.68,
WPDYRQID 3.44, IIRLLEGARP 5.56, DFRPQIDW 4.1, IRGILDSL 5.72,
YIQRNIHH 2.11, VQRFVDGP 5.13, IRSIKDGE 7.61, VIRKVEYI 5.74,
LRFIEAFG 4.06, KDKAEIPV 2.23, PVYRRIDG 3.57, LRHKGEID 5.14,
YHFRNKID 7.01, IRHVAIDY 5.06, IRQRFIDF 4.67, HIRKQVVDQERS 5.57,
LGPEQKELSD 1.82, ALRKQQIE 6.53, LSRFIESG 7.14, LRELVKDH 4.73,
QRYVD 5.81, IRDGLPRQ 1.88, LTRGKQID 6.92, QRQLDTVP 7.7,
YDLRTLTD 4.73, LRQVEWNY 5.99, NEVFAHTQ 1.89, GHRAIDNL 7.15,
DVRVIDSGV 3.73, HSFRQIDQ 8.84, YGDPHAARSL 1.82, YGSRLIDE 6.29,
DFPNRKIE 4.53, GDSEKFE 2.13, LKVRKIVD 5.11, VRQIEGAQ 5.1,
IAARDIEKL 5.19, DPGLGLKL 3.76, DPRHHG 2.06, SIREVDWH 5.05,
IFGQRKLD 5.93, LRATLDVV 5.49, VDSVIHIN 2.41, FDVGRPHA 2,
NKYRRID 6.98, WLRLGLID 5.52, ILLKRLVE 6.44, DINLKNRSIDSS 7.06,
TQRAIDK 8.47, HDSRDRSA 2.67, WASNRLID 5.17, YSRPGHIHIG 2.06,
DFTRELDPA 5.23, DTPRKIDS 5.85, TIRRHVDL 6.57, IDGRRVDL 3.95,
GGILQTWN 1.83, PGRWQLKA 4.07, QRPNIDEL 6.64, RDIVIHYH 1.66,
KLRYEHIDHT 7, VDLYTQKE 2, QLKRKTID 5.81, KFNRLIE 6.56, IDRSVENT 4.55,
KILRIWID 5.44, DVNRLKREIE 6.31, YIIRKDVDV 5.87, HQQRRVD 6.79,
TLRNIETG 6.99, RIRLINDH 7.29, FHRTRYID 6.26, DKKSDAPSIGIE 2.57,
FYQHISLP 1.68, LTRLLDHSP 7.48, LHRWEVDP 4.55, KLPHRLIE 7.31,
HGILRETD 5.16, LRLEIESG 5.33, NWDKHWVY 1.66, IRILIDIS 3.44,
IRIVEAES 5.16, TIRLTDTS 7.5, IKHLAHVA 2.13, RDHSG 2.08,
WQWERLID 5.74, YRRIDGA 5.12, RKEIRDID 6.01, LYRIKIEV 4.31,
GRAIEPVW 5.43, KITSREIE 6.55, GVRQAVG 1.89, HDRLFG 2.12,
LLPRRVE 5.85, LRWAIDFI 3.92, LLRLTEPADT 6.75, QLRFQIHD 7.32,
LARLLDI 5.04, GRHGDHGF 1.84, LYARKVEI 7.06, TDSRINHT 2.02,
FDDIQAQT 2.05, AEILRLLD 6.1, LRKVNDSG 6.48, LRLNVESI 6.6,
VFRGLVDSN 5.55, DGNGQPAH 2.1, PEKALKPS 5.35, DVSIRIID 4.19,
RFVREIE 5.75, LHLRNHID 9.55, DLIAYKQ 2.05, YDYPKYQKESK 2.31,
FRQVEGPVD* 6.9, KSLRFIDV 6.59, QRKIEAIFS 6.28, IRGRKLENEV 5.18,
YGVSRLID 5.15, GLWRQVEG 6.01, SHLNLTLPN 2.11, LLYRNVDG 6.68,
QHRRIEPQ 6.85, FFRLRNVD 6.15, LFRNGIDA 6.25, LHFVRKIE 7,
AAAGDKPSL# 3.67, IRDLFIDG 4.16, EVGVKEVKTKV 4.94, FDSHTNTK 2.15,
HHRIRQLD 6.82, YFREIIDF 3.65, LRTAVDS 7.07, DQLPKYVFS 1.97,
LARGLIDR 7.3, GDGNIVR 2.18, WKESHTTL 2.15, NRAIDWPS 5.22,
QIRDLDPY 5.79, FRAVDPDGDG 7.22, DLRQNLEET 3.28, HWIRRIVE 4.34,
IRLVDILEQNP 4.86, HTLRAVEL 6.38, ARDIDEYD 5.51, AWRSIDEGG 5.18,
WQTRAIDW 4.78, AQLRSVDPATF 6.1, FIARLIDL 5.23, YRRLIDQ 7.34,
FRELDSFL 5.15, RSHGIHHI 1.79, PIRIVDEI 3.99, RWEREID 5.17,
DLRRIPEV 3.23, WHWIRRVE 6.3, SLQKFQDG 2.13, IRNLLDVQ 5.84,
FKLRLIWD 5.6, LRLIYED 6.35, TLRLLED 5.88, PRLIDG 3.39, ARLLDG 5.14,
LRHFAIDT 5.02, IRLNISDV 5.2, QIRADIDN 7.53, SAFRKLDE 5.14,
VIHGDNVH 2.02, DGRLFD 3.89, AHTGALHG 1.96, FDYNESKT 2.05,
LRYFQIEE 5.48, YDQRKVEYS 3.6, IDRRGEVD 4.85, ISRRLDG 5.85,
DYLRVVEQ 4.01, GYSHQGHV 1.6, LRLKITELDK 5.65, QRDKFIDQ 4.55,
RWLRRLDP 7.09, EYRSIDTS 5.2, AAQKDRLV 1.77, GQLREHLD 5.28,
LLQRKVE 8.33, DLDQFLRKRIE 5.37, GQRLVDAV 7.39, IADTHHYP 1.99,
GFQHWNLG 1.97, RLENRWID 4.59, IRDQLDPK 5.77, NPIREIEE 5.35,
LKRAADLVE 5.01, SRIGDYPY 1.81, AARLRLLE 4.97, FATRQLID 5.51,
YFKWRELD 5.36, QEIYNGKP 4.29, IDRTAVDN 3.27, DGYQQYQY 1.89,
LRFFDPAEG* 4.85, IVRRQLDG 7.15, LRSLVDLGPSW 4.55, HFRAVDPDGDG 7.3,
LPPGKDYK 4.53, IKIRRKVDINP 6.17, FQRIAIDE 7.2, FRLFD 5.06,
LKRELLDEG 5.17, LQDRHRHV 1.9, QIREIEQK 7.61, AWRSIDEAG 5.35,
LRVLDDEDS 6.17, GDRELDPV 3.5, LQRSLDEI 6.37, LHTAHNGL 1.69,
DIKKPDS 2.19, HETHRYHT 2.22, GIDSKITE 2.26, QAEREIDG 7.49,
ILRSNAHIDES 7.02, LFYRHRVD 5.69, LIFRLGID 4.6, DVQNFVQY 1.82,
DKEHGEAV 2.09, LIHEVTK 1.99, GQRSRIDY 6.39, KIRVHEIDE 5.83,
PQVGKEWK 4.62, IRLLFDG 5.06, ALDRETDP 4.17, YDYKKNHF 2.09,
YNPVRQID 5.56, IIRYKVEA 5.63, LERAIESL 7.17, LRDRIHDA 5.97,
PVGKEKRV 4.05, IKIRRRVDT 7.49, RGSRQIDA 7.09, WLNRSLDP 7.6,
NLERAIE 8.04, LREKVEYF 6.31, LSREDIDQ 5.99, QRQDIDRI 5.34,
IAGPRTID 5.53, DVEGRSAH 2.15, PGGRDALKS 3.76, IRQQIEYK 6.7,
GTYHLVHA 1.8, INNRQIDK 9.17, DTKTVVEF 2.41, ERLIDLNT 5.68,
YELRHKVD 6.62, YHKSGNTSLES 2.22, NRRKIDGV 7.64, AAAGDKPAP# 4.17,
LFRRHLD 6.64, LDYGKIDH 1.93, IARRQNIE 6.6, IALDRLLD 5.77,
GRLVDSV 6.21, YLRLVNLD 5.04, IRLVTEEL 4.65, IFGVRFID 5.42,
GLRIIEPF 3.95, ALRRLYTDIQEP 4.72, LRLPIEAI 4.54, LRWIEKDG 6.67,
YHHVVQP 2.02, LYRKLEI 6.2, IRADIDKK 8.08, WRLWRQVE 6.6,
LFRELEDA 5.54, YLWRTIDQ 7.77, VWHTGVVG 2, PNGTAVK 4.53,
FRLVRQLD 6.12, DTVGAWTY 1.87, WQKRNIDD 6.72, GARLLDG 5.38,
RYLRRQRVDVS 6.65, DYNHHDVK 2.27, LVRDIWDV 4.65, RSRQIDL 6.96,
VGPGLETK 3.56, RYDNYRHQ 2.06, LRPVEPESEFV 5.16, NIRLPIDA 5.66,
GQNERSID 6.03, DHNHLQQN 1.69, DYWIQQHT 1.98, LDLRSIKEVDE 5.13,
RDRHLHQN 1.76, LARAVEA 6.98, IRQLDPQH 5.96, RLQRNIE 6.75,
TQRFID 6.51, NLKRLLDQGE 7.46, HFLRSIEPVASKV 7.61, DIDARKVE 3.19,
ILHHDEQG 2.09, LRSLDYEALQG 5.41, ISRLLDS 6.23, LRDTDSFY 4.89,
KNPLRAVD 6.46, LLRYVEDG* 5.31, EAHRASHI 2.03, VYQSFDVT 1.85,
INREEIDG 6.86, DLSNTFHQ 2.21, IARRIDKV 6.92, IRKRIIES 6.03,
IYGRGVEY 2.05, LWRLIKDQ 7.52, IRNDKIDH 4.85, RRLIDLGV 6.64,
IRLLNIE 5.18, LRDYDDID 5.14, LRPLLIDG 4.86, YQPGGGH 1.87,
LRTEVETYV 5.74, LRRLDLGE 4.97, GVHPAIA 2.63, GDSAYVLP 2.25,
LDRIIDI 5.03, LRSNEIDS 4.9, IWFQVGVE 1.59, STYQHYAI 1.83,
RLEEGHRQ 3.2, AIYWNGVF 2.26, IFVRALDGG 6.59, YIYRSVEP 6.45,
HPGSETKL 4.25, KKPRGHEH 1.99, FIRALDAF 5.45, VPRKVDG 5.66,
KFRQIED 7.07, IAGRVEID 4.69, LKRELLDE 4.95, DIRSGKID 3.98,
FARLVDDF 5.03, GVYHKLSD 2.34, IYRRIEGK 6.88, DIKKEEAT 2.32,
GDDKSRSI 2.14, QRAIDKITS 7.93, WIREFIDR 5.58, LRDNAIDEG 7.88,
TKRREIDL 6.92, SPHQGSFT 2.07, DSGFHVES 2.17, DSPGFAFK 2.68,
WDDAKHHVS 2.07, NISRYIEP 5.89, ASHGHIHS 2.45, QRSTIDIDES 6.42,
IVIRKKIE 5.16, PGKSDKIS 3.64, IRKIVVDI 3.5, DGDSSSAFQLG 2.13,
LRANWNID 6.68, TQYARDID 5.41, VKYQGDNA 2.33, ILRSDAHIDESNS 8.1,
GRDNSYSI 2.11, EWIRKVVD 4.53, LSRQFDAP 6.1, RHHGGLKE 2.44,
VLRRFD 5.18, IERSEIDQFV 6.87, RLLRLVWD 4.14, ELREVYDY 4.83,
HLRYIIDT 6.1, ELLRRVDAE 6.18, AHKKSHEES 2.99, LVREAVDA 4.4,
IAYDHVVS 1.9, QKRLIDDL 6.81, VRKFIE 4.95, LWRQVDNW 5.26,
LQRETDIG 5.72, AQRYNIDV 6.74, NIGIHKDN 2.32, NLSREINDS 5.55,
LRQLEFPE 5, IDRSVEWK 3.32, SLGKETKKE 5.73, NDSSHFRP 1.98,
HIRVAIDP 5.01, ILRSDAHIDESYS 7.19, IVVDRDID 5.19, LRIKIHEGYE 4.47,
YKIRLDIDNV 5.47, GNDGNKRV 2.07, KLSRFIE 6.43, NLERRIEI 5.72,
AAIRAIES 5.65, HQREVELP 7.72, EIRGLIEEV 5.98, SDVIREVD 5.33,
RDSRLVG 1.94, IRADIDK 7.92, SQREVDLEA 7.55, KLSPDAQN 2.21,
PGTHLKPS 4.1, DSPSYAYG 2.41, LRSLDRNLPSD 4.47, LARIVDPY 5.35,
RDQRKLDE 4.3, GRLDHFTH 1.45, LRHLTDWG 5.31, LRDSWQID 3.34,
AHALSTVV 2.05, LFRDWIDGV 4.25, DLERKIQDLNLS 4.83, LRAVDQSVL 6.91,
LEKKREVD 6.5, TSLRWIDS 6.41, TIRGIDSD 7.88, QNRRQVDF 5.69,
WGDIVQQS 2, LNQWRALD 5.83, HKAIHEQV 2.99, ILIVRAVE 5.58,
LRSPQIED 5.15, GRLEIDTS 5.1, TDTIYYK 2.01, GPSAAQPSRNG 1.57,
GVIPRKVD 5.1, LDHHTHHI 1.83, DLDRFDVD 3.53, KLRGIDPL 7.92,
GHGENQYN 2.02, ILRSDAHIDESS 7.56, DGKEWTHVSLTG 2.49, DFQAQQQS 2.3,
LRLIVENF 4.06, LRELSDVV 4.02, IKIRRRVDL 6.87, DVQRAEID 4.93,
YLWWRTVD 6.18, QRRLD 6.01, VHRKVDLP 7.01, IDRGHSNP 1.9, KIRAVEE 7.21,
HFKRLIDW 4.99

In addition, 52 V13 library discriminating peptides from the V16 array analysis with t-test p-values<0.0001 which overlapped with V13 library from Example 3 (above) are listed in Table 7 below. These peptides are highlighted in green in FIG. 22. The peptides are ordered by increasing p-values for a t-test of the difference in mean log-transformed intensities of subjects who were Chagas seropositive and mean log-transformed intensities of subjects who were Chagas seronegative. Each unique peptide's sequence is followed by the ratio of the mean seropositive over mean seronegative intensity for that peptide.

TABLE 7
V13 Library Peptide Sequences (in V16) Discriminating Between Chagas Seropositive
Samples from Chagas Seronegative Samples in V16 Array Analysis

LREVDQVDG 15.55, LREVEPWKE 7.86, VRLVDPE 7.66, VVREVDG 6.42,
LRALEPHSE 8.52, FRLVDEG 8.03, VRKVDWEG 4.94, FRQVEGPVD 6.9,
LRFFDPAEG 4.85, LLRYVEDG 5.31, HWLRQVED 6.58, LRKFDVFG 4.56,
QVWRQVDAD 5.29, LRPLEVDG 3.91, LRLNDPSDG 5.19, PGFEQKPAQG 2.81,
LRKSDLSD 3.71, FRKLENDG 4.19, ARGDYYLEG 1.49, LRYLEPADG 3.92,
LREFDYFSE 3.16, FRLLDLSG 4.39, LRKVEAHS 4.32, LARQLDWV 3.4,
LRYVDPAQKRD 3.98, DYSSDQVSG 2.22, QRFAVDADNS 3.8, FREADLED 3.45,
LRKVPVEG 3.21, GRQLDPEG 3.48, LREFHVEG 2.4, FQRAVDNHE 4.01,
QRELDFYALS 2.62, SRQVDPLS 3.07, KQRWVEVDG 2.25, AFRELEASG 2.67,
LRKLSLED 2.55, LRFAEVG 2.64, VRQVDGHEG 2.8, ERLLDYG 2.54,
LRVAEFEG 2.55, VRRVDPYF 2.92, AREFDFYG 2.16, GRDYDAWVS 1.69,
VGKAVK 2.67, YRLVDYQALED 2.4, QRLYDWQP 2.2, NRDFDGPVVD 2.3,
SRSVDPA 2.52, ARDYDGNPFS 1.79, PGKAVYAVS 2.33

Best mean performance under cross-validation was achieved for SVM models with 1,000 input peptides. The mean Area Under the Curve (AUC) of Receiver-Operator Characteristic (ROC) curves generated for models with 1000 input peptides trained and tested in 100 cross-validation trials was 0.98 (95% CI 0.97-0.99). The mean sensitivity at a diagnostic threshold selected for 90% specificity was 96% (92%-98%) for these models. The mean specificity at a diagnostic threshold selected for 90% sensitivity was 98% (92%-100%).

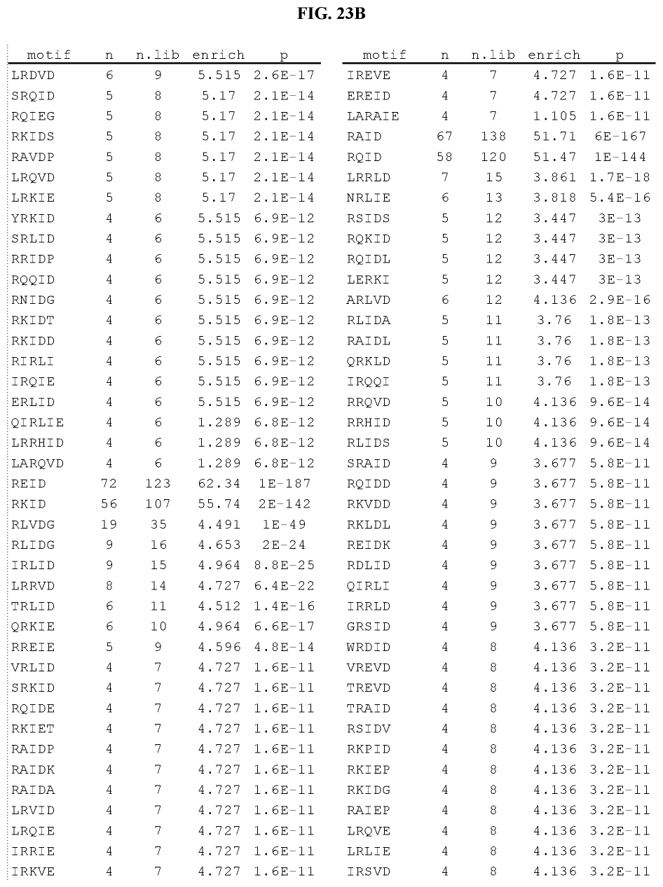

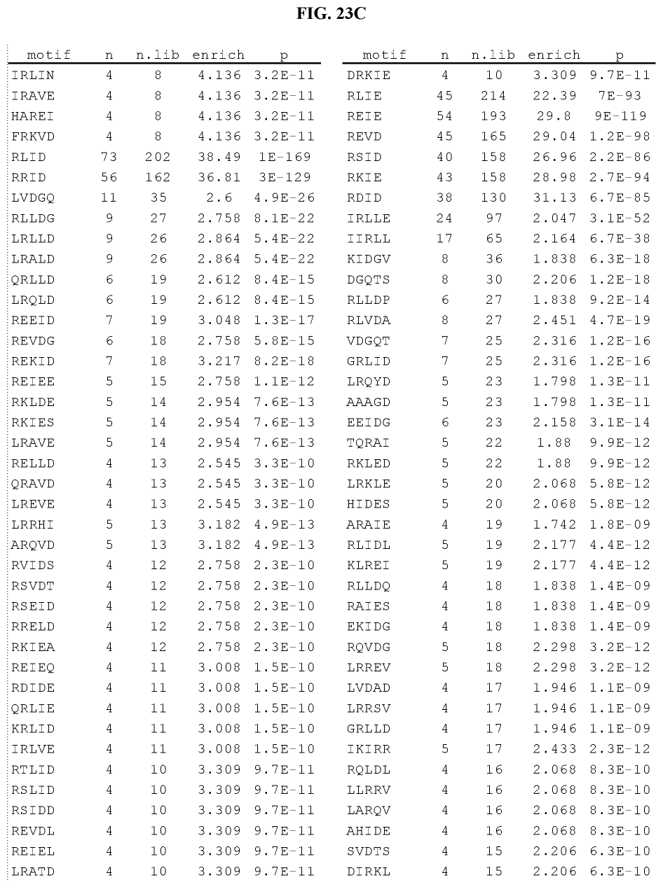

The peptides in the V16 array that discriminated Chagas seropositive from Chagas seronegative samples were found to be enriched in one or more motifs listed in FIG. 23A, FIG. 23B and FIG. 23C relative to the incidence of the same motifs in the entire V16 peptide library.

### Example 8—Proteome Mapping the Chagas-Classifying Peptides Identified on the Extended Array

The 2,707 library peptides that significantly distinguished Chagas positive from negative donors meeting the Bonferroni criterion 95% confidence level were aligned to the T. cruzi proteome with a modified BLAST algorithm and scoring system that used a sliding window of 20-mers (Example 1). This yielded a ranked list of candidate protein-target regions shown in Table 8. These classifying peptides display a high frequency of alignment scores that greatly exceed the maximum scores obtained by performing the same analysis with ten equally-sized sets of peptides that were randomly selected from the library. For example, the maximum score obtained with the randomly selected peptides ranged from less than 8543 to 15920, whereas the classifying peptides generated an alignment score of 46985 to the top hit, Wee90. Thus, in this instance, the classifying peptides provided a protein score that was at least 300% greater than that of the highest scoring random peptide. Reliable results can also be achieved with a lesser degree of separation.

TABLE 8
Top ranking alignments of classifying library peptides
to T. cruzi proteome.

| | | | Amino acid |
|---|---|---|---|
| Rank | T. cruzi protein | UniProt ID | position |
| 1 | Protein kinase Wee90 (Serine/ | K4E3I6 | 520-530 |
| | threonine protein kinase, putative) | | |
| 2 | Uncharacterized protein | K4E3D2 | 440-450 |
| 3 | Uncharacterized protein | Q4E3T5 | 610-620 |
| 4 | Uncharacterized protein | K4DM29 | 540-550 |
| 5 | Mucin TcMUCII, putative | Q4D4I0 | 160-170 |
| 6 | Ubiquitin hydrolase, putative | Q4E0K4 | 50-60 |
| 7 | Dynein intermediate chain, putative | Q4D4E6 | 640-650 |
| 8 | Uncharacterized protein | Q4DSF4 | 400-410 |
| 9 | Microtubule-associated protein Gb4, | Q4DN34 | 100-110 |
| | putative | | |
| 10 | Uncharacterized protein | K4E498 | 90-100 |
| 11 | Kinesin-like protein | K4E5W8 | 700-710 |

These data show that array peptides that mimic parasitic epitopes were bound differentially by peripheral blood antibodies in Chagas seropositive subjects. These discriminating peptides were mapped to several known immunogenic T. cruzi proteins, and to several previously unknown antigens. These data also show that the peptides share strong motifs, including the “LR” motif previously seen on the V13 (Example 4), and include peptides that target known Chagas epitopes from the IEDB.

This study supports the findings provided in Examples 1-4, and extends the list previously obtained from the study using the V13 array.

What is claimed is:

1. A method of identifying the serological state of a subject having or suspected of having a T. cruzi infection, said method comprising:
(a) contacting a sample from said subject to an array of peptides comprising at least 10,000 different peptides;
(b) detecting the binding of antibodies present in said sample to at least 25 peptides on said array to obtain a combination of binding signals; and
(c) comparing said combination of binding signals to two or more groups of combinations of reference binding signals, wherein at least one of each of said group of combinations of reference binding signals are obtained from a plurality of reference subjects known to be seropositive for said infection, and wherein at least one of each of said group of combinations of reference binding signals are obtained from a plurality of subjects known to be seronegative for said infection, thereby determining the serological state of said subject for T. cruzi.

2. The method of claim 1, further comprising:
(i) identifying a combination of differentiating reference binding signals wherein said differentiating reference binding signals distinguish samples from reference subjects known to be seropositive for said infection from samples from reference subjects known to be seronegative for said infection; and
(ii) identifying a combination of discriminating peptides, wherein said combination of differentiating reference binding signals correspond to the combination of discriminating peptides.

3. The method of claim 2, wherein each of said combination of differentiating reference binding signals is obtained by detecting the binding of antibodies present in a sample from each of said plurality of said reference subjects to at least 25 peptides on the array of peptides comprising at least 10,000 different peptides in step (a) of claim 1.

4. The method of claim 1, wherein said subject having or suspected of having said infection is asymptomatic for said infection.

5. The method of claim 1, wherein said subject having or suspected of having said infection is symptomatic for said infection.

6. The method of claim 1, wherein said subject having or suspected of having said infection and said reference subjects are asymptomatic for any infectious disease.

7. The method of claim 2, wherein said discriminating peptides are comprised of one or more sequence motifs listed in FIG. 9B and FIGS. 23A-23C that are enriched in discriminating peptides among all peptides that contain the motif compared to discriminating peptides among all array peptides by greater than 100%.

8. The method of claim 2, wherein said differentiating peptides are selected from the peptides listed in FIGS. 21A-N, Table 6 and Table 7.

9. The method of claim 1, wherein the binding signal corresponding to the binding of antibodies obtained in step (b) is higher than the reference binding signals obtained from the binding of antibodies from samples of subjects having a score of <1 when using when using an S/CO scoring system.

10. The method of claim 1, wherein said one or more groups of reference subjects that are seronegative for T. cruzii are seropositive for hepatitis B virus (HBV).

11. The method of claim 10, wherein said discriminating peptides are enriched by greater than 100% in one or more sequence motifs listed in FIG. 14A.

12. The method of claim 1, wherein said one or more groups of reference subjects that are seronegative for T. cruzii are seropositive for hepatitis C virus (HCV).

13. The method of claim 12, wherein said discriminating peptides are enriched by greater than 100% in one or more sequence motifs FIG. 15A.

14. The method of claim 1, wherein said one or more groups of reference subjects that are seronegative for T. cruzi are seropositive for West Nile Virus (WNV) infection.

15. The method of claim 14, wherein said discriminating peptides are enriched by greater than 100% in one or more sequence motifs listed in FIG. 16A.

16. A method of identifying the serological state of a subject having or suspected of having a viral infection, said method comprising:
(a) contacting a sample from said subject to an array of peptides comprising at least 10,000 different peptides;
(b) detecting the binding of antibodies present in said sample to at least 25 peptides on said array to obtain a combination of binding signals; and
(c) comparing said combination of binding signals to two or more groups of combinations of reference binding signals, wherein at least one of each of said group of combinations of reference binding signals are obtained from a plurality of reference subjects known to be seropositive for said infection, and wherein at least one of each of said group of combinations of reference binding signals are obtained from a plurality of subjects known to be seronegative for said infection, thereby determining the serological state of said subject.

17. The method of claim 16, further comprising:
(i) identifying a combination of differentiating reference binding signals wherein said differentiating binding signals distinguish samples from reference subjects known to be seropositive for said infection from samples from reference subjects known to be seronegative for said infection; and
(ii) identifying a combination of discriminating peptides, wherein said combination of differentiating reference binding signals correspond to the combination of discriminating peptides.

18. The method of claim 17, wherein said viral infection is an HBV infection, and wherein said one or more groups of reference subjects that are seronegative for HBV and are seropositive for HCV.

19. The method of claim 18, wherein said discriminating peptides comprise one or more sequence motifs that are enriched by greater than 100% from FIG. 17A.

20. The method of claim 17, wherein said viral infection is an HBV infection, and wherein said one or more groups of reference subjects that are seronegative for HBV and are seropositive for WNV.

21. The method of claim 20, wherein said discriminating peptides comprise one or more sequence motifs that are enriched by greater than 100% from FIG. 18A.

22. The method of claim 17, wherein said viral infection is an HCV infection, and wherein said one or more groups of reference subjects that are seronegative for HCV and are seropositive for WNV.

23. The method of claim 22, wherein said discriminating peptides comprise one or more sequence motifs that are enriched by greater than 100% from FIG. 19A.

24. A method for determining the serological state of a subject having or suspected of having at least one of a plurality of infections selected from T. cruzi, HBV, HCV, and WNV, said method comprising:
(a) contacting a sample from a subject suspected of having one of said infections to an array of peptides comprising at least 10,000 different peptides;
(b) detecting the binding of antibodies present in said sample to at least 25 peptides on said array to obtain a combination of binding signals;
(c) providing at least a first, a second, a third and a fourth set of differentiating binding signals corresponding to an infection from T. cruzi, HBV, HCV and WNV, wherein each of said set of differentiating binding signals distinguishes samples from a group of subjects being seropositive for one of said infections from a mixture of samples obtained from subjects each being seropositive for one of the remainder of said plurality of infections;
(d) combining said sets of differentiating binding signals to obtain a multiclass set of differentiating binding signals, wherein said multiclass set is capable of differentiating each of said T. cruzi, HBV, HCV and WNV infections from each other; and
(e) comparing said combination of binding signals obtained in step (b) from said subject to said multiclass set of differentiating binding signals, thereby identifying the serological state of said subject.

25. The method of claim 24, further comprising identifying a set of discriminating peptides for each of said first, second, third, and at least fourth set of differentiating binding signals.

26. The method of claim 25, wherein said first set of discriminating peptides display signals that distinguish samples that are seropositive for T. cruzii from a mixture of samples that each are seropositive for one of HBV, HCV, and WNV.

27. The method of claim 26, wherein said discriminating peptides comprise one or more sequence motifs listed in FIG. 10A, that are enriched by greater than 100% when compared to the at least 10,000 peptides in said array.

28. The method of claim 25, wherein said second set of discriminating peptides display signals that distinguish samples that are seropositive for HBV from a mixture of samples that each are seropositive for one of T. cruzii, HCV, and WNV.

29. The method of claim 28, wherein said discriminating peptides comprise one or more sequence motifs listed in FIG. 11A, that are enriched by greater than 100% when compared to the at least 10,000 peptides in said array.

30. The method of claim 25, wherein said third set of discriminating peptides display signals that distinguish samples that are seropositive HCV from a mixture of samples that each are seropositive for one of HBV, T. cruzii and WNV.

31. The method of claim 30, wherein said discriminating peptides comprise one or more sequence motifs listed in FIG. 12A, that are enriched by greater than 100% when compared to the at least 10,000 peptides in said array.

32. The method of claim 25, wherein said at least fourth set of discriminating peptides distinguishes samples that are seropositive for WNV from a mixture of samples that each are seropositive for one of HBV, HCV, and T. cruzii.

33. The method of claim 32, wherein said discriminating peptides comprise one or more sequence motifs listed in FIG. 13A, that are enriched by greater than 100% when compared to the at least 10,000 peptides in said array.

34. The method of claim 25, wherein said differentiating peptides comprise one or more motifs selected from the list in FIG. 20A, that are enriched by greater than 100% when compared to the at least 10,000 peptides in said array.

35. The method of any one of claims 1, 16 and 24, wherein the method performance is characterized by an area under the receiver operator characteristic (ROC) curve (AUC) equal or greater than 0.93.

36. A method for identifying at least one candidate biomarker for an infectious disease in a subject, the method comprising:
(a) providing a peptide array and incubating a biological sample from said subject to the peptide array;
(b) identifying a set of discriminating peptides bound to antibodies in the biological sample from said subject, the set of discriminating peptides displaying binding signals capable of differentiating samples that are seropositive for said infectious disease from samples that are seronegative for said infectious disease;
(c) querying a proteome database with each of the peptides in the set of discriminating peptides;
(d) aligning each of the peptides in the set of discriminating peptides to one or more proteins in the proteome database of the pathogen causing said infectious disease; and
(e) obtaining a relevance score and ranking for each of the identified proteins from the proteome database;
wherein each of the identified proteins is a candidate biomarker for the disease in the subject.

37. The method of claim 36, further comprising obtaining an overlap score, wherein said score corrects for the peptide composition of the peptide library.

38. The method of claim 36, wherein said discriminating peptides are identified as having p-values of less than 10⁻⁷.

39. The method of claim 36, wherein the step of identifying said set of discriminating peptides comprises:
(i) detecting the binding of antibodies present in samples form a plurality of subjects being seropositive for said disease to an array of different peptides to obtain a first combination of binding signals;
(ii) detecting the binding of antibodies to a same array of peptides, said antibodies being present in samples from two or more reference groups of subjects, each group being seronegative for said disease, to obtain a second combination of binding signals;
(iii) comparing said first to said second combination of binding signals; and
(iv) identifying said peptides on said array that are differentially bound by antibodies in samples from subjects having said disease and the antibodies in said samples from two or more reference groups of subjects, thereby identifying said discriminating peptides.

40. The method of claim 36, wherein the number of discriminating peptides corresponds to at least a portion of the total number of peptides on said array.

41. The method of claim 36, wherein said infectious disease is Chagas disease.

42. The method of claim 36, wherein said at least one candidate protein biomarker is selected from the list provided in Table 2.

43. The method of claim 36, wherein said at least one protein biomarker is identified from at least a portion of the discriminating peptides provided in FIG. 21A-N, Table 6 and Table 7.

44. A peptide array comprising at least a portion of the peptides provided in FIGS. 21A-N, Table 6 and Table 7.

45. A method for identifying at least one candidate biomarker for Chagas disease in a subject, the method comprising:
(a) providing a peptide array and incubating a biological sample from said subject to the peptide array;
(b) identifying a set of discriminating peptides bound to antibodies in the biological sample from said subject, the set of discriminating peptides displaying binding signals capable of differentiating samples that are seropositive for said infectious disease from samples that are seronegative for Chagas disease;
(c) querying a proteome database with each of the peptides in the set of discriminating peptides;
(d) aligning each of the peptides in the set of discriminating peptides to one or more proteins in the proteome database of the pathogen causing Chagas disease; and
(e) obtaining a relevance score and ranking for each of the identified proteins from the proteome database;
wherein each of the identified proteins is a candidate biomarker for Chagas disease in the subject.

46. The method of claim 45, further comprising obtaining an overlap score, wherein said score corrects for the peptide composition of the peptide library.

47. The method of claim 45, wherein said discriminating peptides are identified as having p-values of less than 10⁻⁷.

48. The method of claim 45, wherein the step of identifying said set of discriminating peptides comprises:
(i) detecting the binding of antibodies present in samples form a plurality of subjects being seropositive for said disease to an array of different peptides to obtain a first combination of binding signals;
(ii) detecting the binding of antibodies to a same array of peptides, said antibodies being present in samples from two or more reference groups of subjects, each group being seronegative for said disease, to obtain a second combination of binding signals;
(iii) comparing said first to said second combination of binding signals; and
(iv) identifying said peptides on said array that are differentially bound by antibodies in samples from subjects having Chagas disease and the antibodies in said samples from two or more reference groups of subjects, thereby identifying said discriminating peptides.

49. The method of claim 45, wherein the number of discriminating peptides corresponds to at least a portion of the total number of peptides on said array.

50. The method of claim 45, wherein said at least one candidate protein biomarker is selected from the list provided in Table 6.

51. The method of claim 45, wherein said at least one protein biomarker is identified from at least a portion of the discriminating peptides provided in FIGS. 21A-N, Table 6 and Table 7.

52. The method of claim 45, wherein said discriminating peptides are enriched by greater than 100% in one or more sequence motifs listed in FIG. 23.

53. A peptide array comprising peptides that include one or more motifs provided in FIG. 23.

54. The method of any one of claims 1, 16, 24, 36 and 45, wherein the subject is human.

55. The method of any one of claims 1, 16, 24, 36 and 45, wherein the sample is a blood sample.

56. The method of claim 37, wherein the blood sample is selected from whole blood, plasma, or serum.

57. The method of any one of claims 1, 16, 24, 36 and 45, wherein the sample is a serum sample.

58. The method of any one of claims 1, 16, 24, 36 and 45, wherein the sample is a plasma sample.

59. The method of any one of claims 1, 16, 24, 36 and 45, wherein the sample is a dried blood sample.

60. The method of any one of claims 1, 16, 24, 36 and 45, wherein the array of peptides comprises at least 50,000 different peptides.

61. The method of any one of claims 1, 16, 24, 36 and 45, wherein the peptide array comprises at least 100,000 different peptides.

62. The method of any one of claims 1, 16, 24, 36 and 45, wherein the peptide array comprises at least 300,000 different peptides.

63. The method of any one of claims 1, 16, 24, 36 and 45, wherein the peptide array comprises at least 500,000 different peptides.

64. The method of any one of claims 1, 16, 24, 36 and 45, wherein the peptide array comprises at least 1,000,000 different peptides.

65. The method of any one of claims 1, 16, 24, 36 and 45, wherein the peptide array comprises at least 2,000,000 different peptides.

66. The method of any one of claims 1, 16, 24, 36 and 45, wherein the peptide array comprises at least 3,000,000 different peptides.

67. The method of any one of claims 1, 16, 24, 36 and 45, wherein the different peptides on the peptide array is at least 5 amino acids in length.

68. The method of any one of claims 1, 16, 24, 36 and 45, wherein the different peptides on the peptide array are between 5 and 13 amino acids in length.

69. The method of any one of claims 1, 16, 24, 36 and 45, wherein the different peptides are synthesized from less than 20 amino acids.

70. The method of any one of claims 1, 16, 24, 36 and 45, wherein the different peptides on the array are deposited.

71. The method of any one of claims 1, 16, 24, 36 and 45, wherein the different peptides on the array are synthesized in situ.

72. The method of any one of claims 1, 16, 24, 36 and 45, wherein the method performance is characterized by an area under the receiver operator characteristic (ROC) curve (AUC) equal or greater than 0.6.
